(19) United States
(12) Patent Application Publication
(10) Pub. No.: US 2024/0158745 A1
(43) Pub. Date: May 16, 2024

(54) METHODS OF GENERATING OLIGODENDROCYTE PROGENITOR CELLS AND USE THEREOF

(71) Applicant: LINEAGE CELL THERAPEUTICS, INC., Carlsbad, CA (US)

(72) Inventors: Rami SKALITER, Ness Ziona (IL); Francois BINETTE, Napa, CA (US); Dana HAYOUN NEEMAN, Jerusalem (IL); Assaf BAHAT, Jerusalem (IL); Ofer WISER, Jerusalem (IL)

(21) Appl. No.: 18/548,472

(22) PCT Filed: Mar. 10, 2022
(86) PCT No.: PCT/US22/19847

§ 371 Date: Aug. 30, 2023

Related U.S. Application Data
(60) Provisional application No. 63/159,350, filed on Mar. 10, 2021.

(51) Int. Cl. C12N 5/079; A61K 35/30; A61P 25/00

(52) U.S. Cl. CPC C12N 5/0622; A61K 35/30; A61P 25/00; C12N 2500/38; C12N 2501/11; C12N 2501/115; C12N 2501/135; C12N 2506/02

(57) ABSTRACT

Methods for differentiating human pluripotent stem cells to oligodendrocyte progenitor cells (OPCs) are provided. Also provided are cells and cellular compositions obtained by such methods, and uses of such cells. Further provided are methods and protocols for efficiently differentiating human pluripotent stem cells to OPCs.

### CROSS-REFERENCE TO RELATED APPLICATION

This application claims the benefit of U.S. Provisional Application No. 63/159,350, filed Mar. 10, 2021, which is incorporated herein by reference in its entirety and for all purposes.

### FIELD OF TECHNOLOGY

The present disclosure relates to novel methods for differentiating pluripotent stem cells such as human embryonic stem cells first to neuroectoderm progenitor cells with dorsal spinal cord progenitor phenotype, then further to glial progenitor cells, and further to oligodendrocyte progenitor cells. Also provided are cells and cellular compositions obtained by such methods, and uses of such cells. The present disclosure further relates to cells produced by the methods according to the invention that express one or more markers.

### BACKGROUND

Oligodendrocyte progenitor cells (OPCs) are a subtype of glial cells in the central nervous system (CNS) that arise in the ventricular zones of the brain and spinal cord and migrate throughout the developing CNS before maturing into oligodendrocytes. Mature oligodendrocytes produce the myelin sheath that insulates neuronal axons and remyelinate CNS lesions where the myelin sheath has been lost. Oligodendrocytes also contribute to neuroprotection through other mechanisms, including production of neurotrophic factors that promote neuronal survival (Wilkins et al., 2001 Glia 36(1):48-57; Dai et al., 2003 JNeurosci. 23(13):5846-53; Du and Dreyfus, 2002 JNeurosci Res. 68(6):647-54). Unlike most progenitor cells, OPCs remain abundant in the adult CNS where they retain the ability to generate new oligodendrocytes. Accordingly, OPCs and mature oligodendrocytes derived from OPCs are an important therapeutic target for demyelinating and dysmyelinating disorders (such as multiple sclerosis, adrenoleukodystrophy and adrenomyeloneuropathy), other neurodegenerative disorders (such as Alzheimer's disease, amyotrophic lateral sclerosis, and Huntington's disease) and acute neurological injuries (such as stroke and spinal cord injury (SCI)).

Several protocols have been developed for differentiation of human pluripotent stem cells such as embryonic stem cells (ESCs) and induced pluripotent stem cells (iPSCs) into OPCs that can be used in cellular therapy. While these methods have been successful in generating OPCs from human pluripotent stem cells for research purposes, challenges remain with respect to quality, scalability and cost of goods associated with translating the existing protocols to a clinical commercial-scale production process.

There is a need for improved methods for differentiating pluripotent stem cells into OPCs. Ideally, such methods should be easily scalable to produce sufficient quantities of OPCs for cell therapy applications while consistently and reproducibly producing the targeted cell OPCs with the desired quality attributes.

### BRIEF SUMMARY

In various embodiments described herein, the present disclosure provides, inter alia, robust, reliable protocols for differentiating human pluripotent stem cells such as ESCs and iPSCs into oligodendrocyte progenitor cells (OPCs).

The present disclosure is based, in part, on the discovery that human pluripotent stem cells can be readily and efficiently differentiated into spinal cord OPCs in the absence of ventralization of neuroectoderm-restricted progenitor cells mediated by SHH signaling.

In an aspect, provided herein, is a method for obtaining a population of oligodendrocyte progenitor cells (OPCs) from undifferentiated pluripotent stem cells, the method comprising: a) obtaining a culture of undifferentiated pluripotent human embryonic stem cells (hESCs); b) culturing the undifferentiated pluripotent hESCs for a first time period under culture conditions sufficient to induce differentiation of the hESCs to neuroectoderm cells and to neural progenitor cells; and c) culturing the neural progenitor cells from step b) for a second time period under culture conditions sufficient to differentiate the neural progenitor cells to OPCs.

In embodiments, the method, wherein the pluripotent cells in step b) are cultured on laminin in an adherent tissue culture vessel, or in a suspended complex, or both.

In embodiments, the method, wherein the pluripotent stem cells are human embryonic stem cells (hESCs). In embodiments, the pluripotent stem cells are human induced pluripotent stem cells (hiPSCs).

In embodiments, the method, wherein the undifferentiated hESCs of step b) are cultured in the presence of Dorsomorphin, PD0325901, and RA (retinoic acid). In embodiments, a step of culturing further comprising in the presence of AA (ascorbic acid) and RA (retinoic acid).

In embodiments, the method, wherein the neuroectoderm cells from step b) are cultured in the presence of EGF and hsbFGF (heat stable basic fibroblast growth factor). In embodiments, step of culturing further comprising in the presence of EGF (epidermal growth factor) and PDGF-AA (platelet-derived growth factor AA).

In embodiments, the method, wherein the first time period in step b) is from about 3 days to about 60 days. In embodiments, wherein the first time period is from about 10 days to about 15 days. In embodiments, wherein the first time period is about 14 days.

In embodiments, the method, wherein the second time period from step c) is from about 10 days to about 60 days. In embodiments, wherein the second time period is from about 20 days to about 40 days. In embodiments, wherein the second time period is about 28 days.

In embodiments, the method, wherein the differentiated hESCs in step b) are cryopreserved at about day 14.

In embodiments, the method, wherein the cryopreserved cells are thawed, and wherein the subsequently thawed cells are cultured in any remaining steps of the method.

In embodiments, the method, comprising the step of cryopreserving the neural progenitor cells from step b) at or about the completion of the first time period.

In embodiments, the method, wherein the cryopreserved neuroectoderm cells are thawed and cultured in accordance with step c).

In embodiments, the method, wherein the OPCs of step c) are cryopreserved.

In embodiments, the method, wherein the cryopreserved OPCs are thawed.

In embodiments, the method, wherein the OPCs express one or more markers selected from neural/glial antigen 2 (NG2), platelet-derived growth factor receptor A (PDGFRα), and platelet-derived growth factor receptor B (PDGFR(3).

In an aspect, provided herein, is a method of formulating an oligodendrocyte progenitor cells (OPCs) composition for administration to a subject directly after thawing, said method comprising: (a) suspending the OPCs according to the method above in a cryopreservation media to form a cell suspension, (b) storing the cell suspension at a cryopreservation temperature, and (c) thawing the cryopreserved suspension.

In embodiments, the method, wherein the cryopreservation media comprises one or more of adenosine, dextran-40, lactobionic acid, HEPES (N-(2-Hydroxyethyl) piperazine-N′-(2-ethanesulfonic acid)), sodium hydroxide, L-glutathione, potassium chloride, potassium bicarbonate, potassium phosphate, dextrose, sucrose, mannitol, calcium chloride, magnesium chloride, potassium hydroxide, sodium hydroxide, dimethyl sulfoxide (DMSO), and water.

In an aspect, provided herein, is a pharmaceutical composition for administration to a subject, said composition comprising the OPCs according to the method above and a cryopreservation media.

In embodiments, the pharmaceutical composition, wherein the cryopreservation media comprises one or more of adenosine, dextran-40, lactobionic acid, HEPES (N-(2-Hydroxyethyl) piperazine-N′-(2-ethanesulfonic acid)), sodium hydroxide, L-glutathione, potassium chloride, potassium bicarbonate, potassium phosphate, dextrose, sucrose, mannitol, calcium chloride, magnesium chloride, potassium hydroxide, sodium hydroxide, dimethyl sulfoxide (DMSO), and water.

In an aspect, provided herein, is a method for treating a spinal injury in a subject, the method comprising administering to said subject a therapeutically effective amount of the pharmaceutical composition above.

In embodiments, the method for treating a spinal injury in a subject, wherein the administering comprises administering the composition into or adjacent to a spinal cord injury site.

In embodiments, the method for treating a spinal injury in a subject, wherein administering is by injection.

In embodiments, the method for treating a spinal injury in a subject, wherein the administering is by implantation.

In embodiments, the method for treating a spinal injury in a subject, wherein the administering is by transplantation.

In embodiments, the pharmaceutical composition, wherein the concentration of cells is about 1×10⁶ cells per mL to about 100×10⁶ cells per mL.

In embodiments, the pharmaceutical composition, wherein the pharmaceutical composition is stored at a volume of about 100 microliters to about 1 milliliter.

In embodiments, the pharmaceutical composition, wherein the concentration of cells is 100×10⁶ cells per mL.

In embodiments, the pharmaceutical composition, wherein the pharmaceutical composition is stored at a volume of 250 microliters.

In embodiments, the pharmaceutical composition, wherein the pharmaceutical composition is stored at a volume of 300 microliters.

In embodiments, the pharmaceutical composition, wherein the cryopreservation media is a cryosolution.

In embodiments, the pharmaceutical composition, wherein the cryosolution is CryoStor10 (CS10).

In embodiments, the pharmaceutical composition, wherein the OPCs express one or more markers selected from neural/glial antigen 2 (NG2), platelet-derived growth factor receptor A (PDGFRα), and platelet-derived growth factor receptor B (PDGFRβ).

### BRIEF DESCRIPTION OF THE DRAWINGS

FIG. 1 is a flow chart illustrating an overview of the OPC thaw-and-inject (TAI) harvesting and cryopreservation process of the present disclosure.

FIG. 2 is an illustration of the Geron Process of Records (GPOR process) (bottom) compared to the improved OPC manufacturing process (top) of the present disclosure.

FIG. 3 is a flow chart illustrating the signaling sequence schematic from pluripotent stem cells to oligodendrocyte progenitors, used for establishing the OPC differentiation process of the present disclosure.

FIG. 4 is a flow chart illustrating the flow and timeline of the OPC production process of the present disclosure.

FIG. 5 is a flow chart illustrating the good manufacturing process (GMP) for manufacturing 100 TAI vials of OPC according to the present disclosure.

FIG. 6 shows the OPC morphology of control culture conditions, cells cultured in glial progenitor medium (GPM/E) on day 10 (time=0, day 7) at 10× magnification (left) and 20× magnification (right).

FIG. 7 shows the OPC morphology of cells cultured in 50% GPM/E and 50% N2.1 (without IBMX) on day 3 after treatment at 10× magnification (left) and 20× magnification (right).

FIG. 8 shows the OPC morphology of control culture conditions, cells cultured in GPM/E on day 12 at 10× magnification (left) and 20X magnification (right).

FIG. 9 shows the OPC morphology of cells cultured in 100% N2.1 (with IBMX for 2 days) on day 5 after treatment at 10× magnification (left) and 20× magnification (right).

FIG. 10 shows confocal immunostaining for Hoechst (blue) and MBP (green) on day 13 (fixed cells) for a control system cultured in GPM/E (left) and cells cultured in 100% N2.1 (right).

FIG. 11 is a flowchart of a large batch OPC differentiation protocol.

FIG. 12 shows photos of morphology of cells starting from day 35 and at day 42. The culture of successful and failed groups differs by different morphology. The ER-ICBRD-02 batch of cells are presenting an organized, compact and dense cell morphology and did not acquire the spindle-like ‘failed’ morphology.

FIG. 13 is a flowchart of a thawed cell OPC differentiation protocol.

FIG. 14 shows photos of morphology of ongoing runs are presented compared to the ICB thawed cells, at day 21 aggregates morphology resemblance between origin runs and their derived ICB's is presented.

FIG. 15 shows photos of morphology of ongoing runs are presented compared to the ICB thawed cells, at days 35 and 42. Morphology resemblance between origin runs and their derived ICB's is presented, cells are organized as compact and dense cells.

### DETAILED DESCRIPTION

This description is not intended to be a detailed catalog of all the different ways in which the disclosure may be implemented, or all the features that may be added to the instant disclosure. For example, features illustrated with respect to one embodiment may be incorporated into other embodiments, and features illustrated with respect to a particular embodiment may be deleted from that embodiment. Thus, the disclosure contemplates that in some embodiments of the disclosure, any feature or combination of features set forth herein can be excluded or omitted. In addition, numerous variations and additions to the various embodiments suggested herein will be apparent to those skilled in the art in light of the instant disclosure, which do not depart from the instant disclosure. In other instances, well-known structures, interfaces, and processes have not been shown in detail in order not to unnecessarily obscure the invention. It is intended that no part of this specification be construed to affect a disavowal of any part of the full scope of the invention. Hence, the following descriptions are intended to illustrate some particular aspects of the disclosure, and not to exhaustively specify all permutations, combinations and variations thereof.

Unless otherwise defined, all technical and scientific terms used herein have the same meaning as commonly understood by one of ordinary skill in the art to which this disclosure belongs. The terminology used in the description of the disclosure herein is for the purpose of describing particular embodiments only and is not intended to be limiting of the disclosure.

All publications, patent applications, patents and other references cited herein are incorporated by reference in their entireties.

Unless the context indicates otherwise, it is specifically intended that the various features of the disclosure described herein can be used in any combination. Moreover, the present disclosure also contemplates that in some embodiments of the disclosure, any feature or combination of features set forth herein can be excluded or omitted.

Methods disclosed herein can comprise one or more steps or actions for achieving the described method. The method steps and/or actions may be interchanged with one another without departing from the scope of the present invention. In other words, unless a specific order of steps or actions is required for proper operation of the aspect, the order and/or use of specific steps and/or actions may be modified without departing from the scope of the present invention.

### I. Definitions

As used in the description of the disclosure and the appended claims, the singular forms “a,” “an” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

The terms “about” and “approximately” as used herein when referring to a measurable value such as a percentages, density, volume and the like, is meant to encompass variations off ±10%, ±5%, ±1%, ±0.5%, or even ±0.1% of the specified amount.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

As used herein, “oligodendrocyte progenitor cells” (OPCs) refer to cells found in the central nervous system that are of a neuroectoderm/glial lineage, express the characteristic marker neural/glial antigen 2 (NG2) and are capable of differentiating into oligodendrocytes.

The terms “glial lineage cells,” “glial progenitor cells” and “glial cells” are used interchangeably herein and refer to non-neuronal CNS cells that are derived from neuroectoderm/neural progenitor cells. Glial progenitor cells can be further differentiated to form OPCs/oligodendrocytes or astrocytes. In certain embodiments, the glial progenitor cells of the present disclosure express one or more markers selected from calcium voltage-gated channel auxiliary subunit gamma 4 (CACNG4), fatty acid binding protein 7 (FABP7), and netrin-1 receptor (DCC).

The terms “neuroectoderm,” “neuroectoderm cells,” “neuroectoderm precursor,” “neuroectoderm progenitor,” “neural progenitor” and “neural precursor” are used interchangeably herein and refer to cells that can be differentiated along a neural precursor pathway and that are capable of forming CNS neurons, oligodendrocytes, astrocytes and ependymal cells. In certain embodiments, the neuroectoderm cells of the present disclosure express one or more markers selected from paired box 6 (PAX6), Hes family BHLH transcription factor 5 (HESS) and zinc finger and BTB domain containing 16 (ZBTB16).

As used herein, the terms “dorsal” and “ventral” refer to distinct neural cell subtypes emerging from progenitor cells arrayed into spatially discrete domains along the dorsal-ventral axis of the neural tube in the developing spinal cord. This process, known as dorsal-ventral patterning, is controlled by secreted signals that partition the neural progenitor cells. BMP and Wnt signaling initiate patterning from the dorsal neural tube (Lee and Jessell, 1999 Annu. Rev. Neurosci. 22: 261-294), whereas secretion of SHH has a key role in establishing ventral neuronal cell fates (Chiang et al., 1996 Nature 383: 407-413; Ericson et al., 1996 Cell 87: 661-683; Briscoe et al., 2001 Mol. Cell 7:1279-1291).

The terms “dorsal neuroectoderm progenitor cell,” “dorsal neural progenitor cell” and “dNPC” are used interchangeably herein and refer to a neural progenitor cell that has the dorsal spinal cord phenotype and has been obtained by differentiating pluripotent stem cells to neuroectoderm-restricted precursors in the absence of exogenous SHH or a SHH signaling activator. In certain embodiments, the dNPCs express one or more markers selected from paired box 3 (PAX3), paired box 7 (PAX7) and activating protein 2 (AP2).

As used herein, the term “embryoid body” (EB) refers to a three-dimensional cellular aggregate derived from pluripotent stem cells that has undergone spontaneous differentiation towards all three germ layers. EBs are formed when pluripotent stem cells are removed from culture conditions that inhibit differentiation. For example, in the case of human embryonic stem cells, removal of basic fibroblast growth factor (bFGF) and transforming growth factor beta (TGE13) from the culture media results in spontaneous differentiation towards all three germ layers and formation of EBs.

As used herein, the term “BMP signaling inhibitor” refers to a small molecule or protein modulator that is capable of downregulating signaling along the bone morphogenetic protein (BMP) signaling pathway. In certain embodiments, the BMP signaling inhibitor directly targets Activin A receptor, type I (ACVR1), also known as activin receptor-like kinase 2 (ALK2). In certain embodiments, the BMP signaling inhibitor is selected from the group consisting of Dorsomorphin, DMH-1, K02288, ML347, LDN193189 and Noggin protein.

As used herein, the term “MAPK/ERK inhibitor” refers to a small molecule or protein modulator that inhibits the MAPK/ERK kinase. In certain embodiments, the MAPK/ERK inhibitor is selected from the group consisting of PD0325901, AZD6244, GSK1120212, PD1 84352 and Cobimetinib.

The terms “SHH signaling activator,” “SHH signaling agonist,” “SHH activator” and “SHH agonist” are used interchangeably herein and refer to a small molecule or protein modulator that is capable of activating the Sonic Hedgehog (SHE) signaling pathway. Non-limiting examples of a SHE signaling activator include Purmorphamine (PMA), Smoothened Agonist (SAG, CAS 364590-63-6) and Sonic Hedgehog (SHH) protein.

As used herein, the terms “FGF” or “bFGF” refer to human basic fibroblast growth factor, or FGF-2, which is a growth factor and signaling protein encoded by the FGF2 gene. The terms are also used interchangeably to refer to any sequence variants or conjugates thereof, including commercially available “heat stable” FGF (hs-FGF, hs-bFGF, hs-hbFGF, and/or hsbFGF).

As used herein, the term “undesirable cell types” refers to cells outside of the neuroectoderm lineage that can result in the formation of ectopic tissues upon implantation, or that result in the formation of one or more cysts in a cyst assay, as described herein. In an embodiment, “undesirable cell types” can include epithelial lineage cells such as cells positive for CD49f, a marker expressed by both neural progenitor cells and epithelial cells, or cells positive for CLDN6 or EpCAM, two markers expressed by both pluripotent cells and epithelial cells.

As used herein, “implantation” or “transplantation” refers to the administration of a cell population into a target tissue using a suitable delivery technique, (e.g., using an injection device).

As used herein, a “subject” refers to an animal or a human.

As used herein, a “subject in need thereof” refers to an animal or a human having damaged tissue in the central nervous system. In an embodiment, an animal or a human is experiencing a loss of motor function.

The terms “central nervous system” and “CNS” are used interchangeably herein and refer to the complex of nerve tissues that control one or more activities of the body, which include but are not limited to, the brain and the spinal cord in vertebrates.

As used herein, “treatment” or “treating,” with respect to a condition or a disease, is an approach for obtaining beneficial or desired results including preferably clinical results after a condition or a disease manifests in a patient. Beneficial or desired results with respect to a disease include, but are not limited to, one or more of the following: improving a condition associated with a disease, curing a disease, lessening severity of a disease, delaying progression of a disease, alleviating one or more symptoms associated with a disease, increasing the quality of life of one suffering from a disease, prolonging survival, and any combination thereof. Likewise, for purposes of this disclosure, beneficial or desired results with respect to a condition include, but are not limited to, one or more of the following: improving a condition, curing a condition, lessening severity of a condition, delaying progression of a condition, alleviating one or more symptoms associated with a condition, increasing the quality of life of one suffering from a condition, prolonging survival, and any combination thereof.

### I. OPC Compositions

The methods of the present disclosure can be used to obtain compositions comprising oligodendrocyte progenitor cells (OPCs) that are suitable for cellular therapy. The OPCs obtained according to the present disclosure express a high level of the proteoglycan NG2, PDGFRa, and/or PDGFRβ characteristic of OPCs and low levels of non-OPC markers associated with undesirable cell types, such as CD49f, which can be expressed by both neural progenitor cells and epithelial cells and is associated with in vitro cyst formation (Debnath J, Muthuswamy SK, Brugge JS. Morphogenesis and oncogenesis of MCF-10A mammary epithelial acini grown in three-dimensional basement membrane cultures. 2003 Methods. 3:256-68), cytokeratin 7 (CK7), or CLDN6 and EpCAM, two markers expressed by both pluripotent cells and epithelial cells (Lin D, Guo Y, Li Y, Ruan Y, Zhang M, Jin X, Yang M, Lu Y, Song P, Zhao S, Dong B, Xie Y, Dang Q, Quan C. Bioinformatic analysis reveals potential properties of human Claudin-6 regulation and functions. Oncol Rep. 2017 Aug;38(2):875-885; Huang L, Yang Y, Yang F, Liu S, Zhu Z, Lei Z, Guo J. Functions of EpCAM in physiological processes and diseases (Review). Int JMol Med. 2018 Oct;42(4):1771-1785).

In certain embodiments, the OPCs generated in accordance with the present disclosure are the in vitro differentiated progeny of human pluripotent stem cells. In certain embodiments, the OPCs obtained in accordance with the present disclosure are the in vitro differentiated progeny of human embryonic stem cells. In other embodiments, the OPCs obtained in accordance of the present disclosure are the in vitro differentiated progeny of induced pluripotent stem (iPS) cells.

One or more characteristics of the OPC population obtained can be determined by quantifying various cell markers using flow cytometry, for example, to determine what percentage of the cell population is positive for a particular marker or set of markers or to identify undesirable cell types present in the OPC population.

An OPC population obtained according to the present disclosure can comprise from about 30% to about 100% OPCs, such as at least about 35%, such as at least about 40%, such as at least about 45%, such as at least about 50%, such as at least about 55%, such as at least about 60%, such as at least about 65%, such as at least about 70%, such as at least about 75%, such as at least about 80%, such as at least about 85%, such as at least about 90%, such as at least about 95%, such as at least about 98%, such as at least about 99%, such as at least about 99.5%, such as at least about 99.8%, or such as at least about 99.9% OPCs. The percentage can be any value or subrange within the recited ranges, including endpoints. The percentage of OPCs in the population may be determined, for example, by the presence of positive markers of OPCs and/or the absence of negative markers of OPCs. Examples of OPC positive markers include, without limitation, NG2, PDGFRa, and/or PDGFRβ. Additional examples of OPC markers can be found in WO 2020/154533, which is incorporated by reference herein for all that is disclosed therein, including, for example, markers of OPCs and other cell types, methods of differentiating cells, methods of using differentiated cells, cell types, reagents, and all other methods, compositions, and disclosure.

An OPC population obtained according to the present disclosure can comprise from about 30% to about 100% NG2 positive cells, such as at least about 35%, such as at least about 40%, such as at least about 45%, such as at least about 50%, such as at least about 55%, such as at least about 60%, such as at least about 65%, such as at least about 70%, such as at least about 75%, such as at least about 80%, such as at least about 85%, such as at least about 90%, such as at least about 95%, such as at least about 98%, such as at least about 99%, such as at least about 99.5%, such as at least about 99.8%, or such as at least about 99.9% NG2 positive cells. In certain embodiments, an OPC population obtained according to the present disclosure can comprise from about 45% to about 75% NG2 positive cells, such as about 45% to about 50%, such as about 50% to about 55%, such as about 55% to about 60%, such as about 60% to about 65%, such as about 65% to about 70%, such as about 70% to about 75%, such as about 50% to about 70%, such as about 55% to about 65%, or such as about 58% to about 63% NG2 positive cells. In other embodiments, an OPC population obtained according to the present disclosure can comprise from about 60% to about 90% NG2 positive cells, such as about 60% to about 65%, such as about 65% to about 70% positive cells. The percentage can be any value or subrange within the recited ranges, including endpoints.

An OPC population obtained according to the present disclosure can comprise from about 30% to about 100% PDGFRa positive cells, such as at least about 35%, such as at least about 40%, such as at least about 45%, such as at least about 50%, such as at least about 55%, such as at least about 60%, such as at least about 65%, such as at least about 70%, such as at least about 75%, such as at least about 80%, such as at least about 85%, such as at least about 90%, such as at least about 95%, such as at least about 98%, such as at least about 99%, such as at least about 99.5%, such as at least about 99.8%, or such as at least about 99.9% PDGFRa positive cells. In certain embodiments, an OPC population obtained according to the present disclosure can comprise from about 45% to about 75% PDGFRa positive cells, such as about 45% to about 50%, such as about 50% to about 55%, such as about 55% to about 60%, such as about 60% to about 65%, such as about 65% to about 70%, such as about 70% to about 75%, such as about 50% to about 70%, such as about 55% to about 65%, or such as about 58% to about 63% PDGFRa positive cells. In other embodiments, an OPC population obtained according to the present disclosure can comprise from about 60% to about 90% PDGFRa positive cells, such as about 60% to about 65%, such as about 65% to about 70% positive cells. The percentage can be any value or subrange within the recited ranges, including endpoints.

An OPC population obtained according to the present disclosure can comprise from about 30% to about 100% PDGFRβ positive cells, such as at least about 35%, such as at least about 40%, such as at least about 45%, such as at least about 50%, such as at least about 55%, such as at least about 60%, such as at least about 65%, such as at least about 70%, such as at least about 75%, such as at least about 80%, such as at least about 85%, such as at least about 90%, such as at least about 95%, such as at least about 98%, such as at least about 99%, such as at least about 99.5%, such as at least about 99.8%, or such as at least about 99.9% PDGFRβ positive cells. In certain embodiments, an OPC population obtained according to the present disclosure can comprise from about 45% to about 75% PDGFRβ positive cells, such as about 45% to about 50%, such as about 50% to about 55%, such as about 55% to about 60%, such as about 60% to about 65%, such as about 65% to about 70%, such as about 70% to about 75%, such as about 50% to about 70%, such as about 55% to about 65%, or such as about 58% to about 63% PDGFRβ positive cells. In other embodiments, an OPC population obtained according to the present disclosure can comprise from about 60% to about 90% PDGFRβ positive cells, such as about 60% to about 65%, such as about 65% to about 70% positive cells. The percentage can be any value or subrange within the recited ranges, including endpoints.

In an embodiment, an OPC population obtained according to the present disclosure can be capable of forming less than or equal to four epithelial cysts per 100,000 cells in a cyst assay as described in Example 8 of the present disclosure. In another embodiment, an OPC population obtained according to the present disclosure can be capable of forming less than or equal to three epithelial cysts per 100,000 cells in a cyst assay. In another embodiment, OPC population obtained according to the present disclosure can be capable of forming less than or equal to two epithelial cysts per 100,000 cells in a cyst assay. In yet another embodiment, an OPC population obtained according to the present disclosure can be capable of forming less than or equal to one epithelial cysts per 100,000 cells in a cyst assay as described in Example 8 of the present disclosure.

### II. Pharmaceutical Formulation

OPC compositions in accordance with the present disclosure can further comprise a pharmaceutically-acceptable carrier. In an embodiment, a pharmaceutically-acceptable carrier can comprise dimethyl sulfoxide (DMSO). In an embodiment, a pharmaceutically-acceptable carrier does not comprise dimethyl sulfoxide. As mentioned above, a composition can be further adapted for cryopreservation at or below −80° C. to −195° C. In embodiments, a composition can be formulated to thaw and administered directly into a subject, e.g. via injection, without additional manipulation prior to administration. In embodiments, a composition can be formulated including a cryosolution such as CryoStor® 10 (CS10) as a cryopreservation media. In an embodiment, a composition can be filtered using a 60 um filter kit before cryopreservation.

OPC compositions in accordance with the present disclosure can be formulated for administration via a direct injection to the spinal cord of a subject. In an embodiment, an OPC composition in accordance with the present disclosure can be formulated for intracerebral, intraventricular, intrathecal, intranasal, or intracisternal administration to a subject. In an embodiment, an OPC composition in accordance with the present disclosure can be formulated for administration via an injection directly into or immediately adjacent to an infarct cavity in the brain of a subject. In an embodiment, a composition in accordance with the present disclosure can be formulated for administration through implantation. In an embodiment, a composition in accordance with the present disclosure can be formulated as a solution.

An OPC composition in accordance with the present disclosure can comprise from about 1×10⁶ to about 200×10⁶ cells per milliliter, such as about 1×10⁶ cells per milliliter, such as about 2×10⁶ cells per milliliter, such as about 3×10⁶ cells per milliliter, such as about 4×10⁶ cells per milliliter, such as about 5×10⁶ cells per milliliter, such as about 6×10⁶ cells per milliliter, such as about 7×10⁶ cells per milliliter, such as about 8×10⁶ cells per milliliter, such as about 9×10⁶ cells per milliliter, such as about 10×10⁶ cells per milliliter, such as about 20×10⁶ cells per milliliter, such as about 30×10⁶ cells per milliliter, such as about 40×10⁶ cells per milliliter, such as about 50×10⁶ cells per milliliter, such as about 60×10⁶ cells per milliliter, such as about 70×10⁶ cells per milliliter, such as about 80×10⁶ cells per milliliter, such as about 90×10⁶ cells per milliliter, such as about 100×10⁶ cells per milliliter, such as about 101×10⁶ cells per milliliter, such as about 102×10⁶ cells per milliliter, such as about 103×10⁶ cells per milliliter, such as about 104×10⁶ cells per milliliter, such as about 105×10⁶ cells per milliliter, such as about 106×10⁶ cells per milliliter, such as about 107×10⁶ cells per milliliter, such as about 108×10⁶ cells per milliliter, such as about 109×10⁶ cells per milliliter, such as about 110×10⁶ cells per milliliter, such as about 120×10⁶ cells per milliliter, such as about 130×10⁶ cells per milliliter, such as about 140×10⁶ cells per milliliter, such as about 150×10⁶ cells per milliliter, such as about 160×10⁶ cells per milliliter, such as about 170×10⁶ cells per milliliter, such as about 180×10⁶ cells per milliliter, such as about 190×10⁶ cells per milliliter, such as about 200 10⁶ cells per milliliter. The number of cells can be any value or subrange within the recited ranges.

In yet another embodiment, an OPC composition in accordance with the present disclosure can have a volume ranging from about 250 microliters to about 1 milliliter, such as about 300 microliters, such as about 400 microliters, such as about 500 microliters, such as about 600 microliters, such as about 700 microliters, such as about 800 microliters, such as about 900 microliters, or such as about 1 milliliter In an embodiment, a composition in accordance with the present disclosure can have a volume ranging from about 250 microliters to about 1 milliliters, such as about 250 microliters to about 900 microliters, such as about 300 microliters to about 800 microliters, such as about 400 microliters to about 700 microliters, or such as about 500 microliters to about 600 microliters. In another embodiment, a composition in accordance with the present disclosure can have a volume ranging from about 100 microliters to about 1 milliliter, such as about 200 microliters to about 900 microliters, such as about 300 microliters to about 800 microliters, such as about 400 microliters to about 700 microliters, or such as about 500 microliters to about 600 microliters. The volume can be any value or subrange within the recited ranges. In an embodiment, an OPC composition in accordance with the present disclosure can be in a container configured for cryopreservation or for administration to a subject in need thereof. In an embodiment, a container can be a prefilled syringe.

In an embodiment, an OPC composition in accordance with the present disclosure can be administered at a volume ranging from about 50 microliters to about 100 microliters, such as about 55 microliters, such as about 60 microliters, such as about 65 microliters, such as about 70 microliters, such as about 75 microliters, such as about 80 microliters, such as about 85 microliters, such as about 90 microliters, such as about 95 microliters, or such as about 100 microliters. In an embodiment, an OPC composition in accordance with the present disclosure can be administered at a volume ranging from about 50 microliters to about 100 microliters, such as about 55 microliters to about 95 microliters, such as about 60 microliters to about 90 microliters, such as about 65 microliters to about 85 microliters, or such as about 70 microliters to about 80 microliters. The volume can be any value or subrange within the recited ranges.

In an embodiment, an OPC composition in accordance with the present disclosure can be administered at a volume ranging from about 5.0×10⁶ cells to about 10.0×10⁶ cells, such as about 5.5×10⁶ cells, such as about 6.0×10⁶ cells, such as about 6.5×10⁶ cells, such as about 7.0×10⁶ cells, such as about 7.5×10⁶ cells, such as about 8.0×10⁶ cells, such as about 8.5×10⁶ cells, such as about 9.0×10⁶ cells, such as about 9.5×10⁶ cells, or such as about 10.0×10⁶ cells. In an embodiment, an OPC composition in accordance with the present disclosure can be administered at a volume ranging from about 5.0×10⁶ cells to about 10.0×10⁶ cells, such as about 5.5×10⁶ cells to about 9.5×10⁶ cells, such as about 6.0×10⁶ cells to about 9.0×10⁶ cells, such as about 6.5×10⁶ cells to about 8.5×10⁶ cells, or such as about 7.0×10⁶ cells to about 8.0×10⁶ cells. The cell number can be any value or subrange within the recited ranges.

### III. Methods of Use

An OPC composition obtained in accordance with the present disclosure can be used in cellular therapy to improve one or more neurological functions in a subject in need of treatment. In an embodiment, an OPC cell population in accordance with the present disclosure can be injected or implanted into a subject in need thereof. In an embodiment, a cell population in accordance with the present disclosure can be implanted into a subject in need thereof for treating spinal cord injury, stroke, or multiple sclerosis.

In an embodiment, a cell population in accordance with the present disclosure can be capable of inducing myelination of denuded axons at an implantation site in a subject. In an embodiment, a cell population generated in accordance with a method of the present disclosure can exhibit improved capacity for engraftment and migration. In an embodiment, a cell population generated in accordance with a method of the present disclosure can be capable of improving post-injury repair or regeneration of neural tissue in a subject.

A cell population in accordance with the present disclosure can be capable of improving a sensory function in a subject in need of therapy following implantation of the population into the subject. Improvements in a sensory function can be evaluated using the International Standards for Neurological Classification of Spinal Cord Injury (ISNCSCI) Exam, such as determining sensory levels for right and left sides for pin prick and light touch sensations. A cell population in accordance with the present disclosure can be capable of improving a motor function in a subject in need of therapy following implantation of the population into the subject. An improved motor function can be evaluated using the ISNCSCI Exam, such as determining motor levels for right and left sides for total paralysis, palpable or visible contraction, active movement, full range of motion against gravity, and sufficient resistance.

A cell population in accordance with the present disclosure can be capable of reducing a volume of an injury-induced central nervous system parenchymal cavitation in 12 months or less. In an embodiment, a cell population in accordance with the present disclosure can be capable of reducing a volume of an injury-induced central nervous system parenchymal cavitation in a subject in 6 months or less, 5 months or less, 4 months or less, 3 months or less, 2 months or less, or less than 1 month.

Use in treatment of CNS traumatic injury. In certain embodiments, a cell population in accordance with the present disclosure can be capable of engrafting at a spinal cord injury site following implantation of a composition comprising the cell population into the spinal cord injury site.

In certain embodiments, a cell population in accordance with the present disclosure is capable of remaining within the spinal cord injury site of the subject for a period of about 90 days or longer following implantation of a dose of the composition into the spinal cord injury site. In other embodiments, a cell population in accordance with the present disclosure is capable of remaining within the spinal cord injury site of the subject for a period of about 1 year or longer following implantation of a dose of the composition into the spinal cord injury site. In further embodiments, a cell population in accordance with the present disclosure is capable of remaining within the spinal cord injury site of the subject for a period of about 2 years or longer following implantation of a dose of the composition into the spinal cord injury site. In further embodiments, a cell population in accordance with the present disclosure is capable of remaining within the spinal cord injury site of the subject for a period of about 3 years or longer following implantation of a dose of the composition into the spinal cord injury site. In further embodiments, a cell population in accordance with the present disclosure is capable of remaining within the spinal cord injury site of the subject for a period of about 4 years or longer following implantation of a dose of the composition into the spinal cord injury site. In yet further embodiments, a cell population in accordance with the present disclosure is capable of remaining within the spinal cord injury site of the subject for a period of about 5 years or longer following implantation of a dose of the composition into the spinal cord injury site.

In certain embodiments, a cell composition in accordance with the present disclosure is capable of improving upper extremity motor function in a human subject with a spinal cord injury when administered to said subject. In certain embodiments, the subject has a cervical spinal cord injury. In other embodiments, the subject has a thoracic spinal cord injury.

In one embodiment, the present disclosure provides a method of improving upper extremity motor function in a human subject with a spinal cord injury, comprising administering to said subject a composition that comprises a population of allogeneic human oligodendrocyte cells that are capable of engrafting at a spinal cord injury site. In certain embodiments, administering the composition comprises injecting the composition into the spinal cord injury site. In some embodiments, the composition is injected approximately 2-10 mm caudal of the spinal cord injury epicenter. In further embodiments, the composition is injected approximately 5 mm caudal of the spinal cord injury epicenter. In some embodiments, the subject has a cervical spinal cord injury. In other embodiments, the subject has a thoracic spinal cord injury.

In certain embodiments, the subject to whom a composition comprising a population of allogeneic human oligodendrocyte cells is administered to according to the methods of the present disclosure, gains an improvement in upper extremity motor function equal to at least one motor level (as defined based on International Standards for Neurological Classification of Spinal Cord Injury [ISNCSCI]). The improvement in function may be unilateral or bilateral. In other embodiments, the subject to whom a composition comprising a population of allogeneic human oligodendrocyte cells is administered to according to the methods of the present disclosure, gains an improvement in upper extremity motor function equal to at least two motor levels either unilaterally or bilaterally. In certain embodiments, the subject gains an improvement in upper extremity motor function equal to at least one motor level on one side and equal to at least two motor levels on the other side. In certain embodiments, the subject exhibits an improved upper extremity motor score (UEMS) relative to the subject baseline score prior to administration of a population of allogeneic human oligodendrocyte cells according to the methods of the present disclosure.

In certain embodiments, a cell composition in accordance with the present disclosure is formulated for delivery or administration into the spine parenchyma, or though peripheral perfusion adjacent to or at the injury site under the dura, the arachnoid, or pia mater.

### IV. Propagation and Culture of Undifferentiated Pluripotent Stem Cells

Differentiation of pluripotent stem cells in accordance with the present disclosure can be carried out using any suitable pluripotent stem cells as a starting material. In one embodiment, a method can be carried out on a human embryonic stem cell (hESC) line. In another embodiment, a method can be carried out using induced pluripotent stem cells (iPSCs). In another embodiment, a method can be carried out using cells that are derived from an H1, H7, H9, H13, or H14 cell line. In another embodiment, a method can be carried out on a primate pluripotent stem (pPS) cell line. In yet another embodiment, a method can be carried using undifferentiated stem cells derived from parthenotes, which are embryos stimulated to produce hESCs without fertilization.

Methods of propagation and culture of undifferentiated pluripotent stem cells have been previously described. With respect to tissue and cell culture of pluripotent stem cells, the reader may wish to refer to any of numerous publications available in the art, e.g., Teratocarcinomas and Embryonic Stem cells: A Practical Approach (E. J. Robertson, Ed., IRL Press Ltd. 1987); Guide to Techniques in Mouse Development (P. M. Wasserman et al., Eds., Academic Press 1993); Embryonic Stem Cell Differentiation in Vitro (M. V. Wiles, Meth. Enzymol. 225:900, 1993); Properties and Uses of Embryonic Stem Cells: Prospects for Application to Human Biology and Gene Therapy (P. D. Rathjen et al., Reprod. Fertil. Dev. 10:31, 1998; and R. I. Freshney, Culture of Animal Cells, Wiley-Liss, New York, 2000). Each of these is hereby incorporated by reference in its entirety.

Undifferentiated pluripotent stem cells can be maintained in an undifferentiated state without added feeder cells (see, e.g., (2004) Rosler et al., Dev. Dynam. 229:259). Feeder-free cultures are typically supported by a nutrient medium containing factors that promote proliferation of the cells without differentiation (see, e.g., U.S. Pat. No. 6,800,480). In one embodiment, conditioned media containing such factors can be used. Conditioned media can be obtained by culturing the media with cells secreting such factors. Suitable cells include, but are not limited to, irradiated (4,000 Rad) primary mouse embryonic fibroblasts, telomerized mouse fibroblasts, or fibroblast-like cells derived from pPS cells (U.S. Pat. No. 6,642,048). Medium can be conditioned by plating the feeders in a serum free medium, such as knock-out DMEM supplemented with 20% serum replacement and 4 ng/mL bFGF. Medium that has been conditioned for 1-2 days can be supplemented with further bFGF, and used to support pPS cell culture for 1-2 days (see. e.g., WO 01/51616; Xu et al., (2001) Nat. Biotechnol. 19:971).

Alternatively, fresh or non-conditioned medium can be used, which has been supplemented with added factors (like a fibroblast growth factor or forskolin) that promote proliferation of the cells in an undifferentiated form. Non-limiting examples include a base medium like X-VIVO™ 10 (Lonza, Walkersville, MD.), or QBSFTM-60 (Quality Biological Inc. Gaithersburg, MD.), or mTeSR1, mTeSR plus, or NutriSTem (Biological Industries, Cromwell, CT) upplemented with bFGF at 40-80 ng/mL, and optionally containing SCF (15 ng/mL), or Flt3 ligand (75 ng/mL) (see, e.g., Xu et al., (2005) Stem Cells 23(3):315). These media formulations have the advantage of supporting cell growth at 2-3 times the rate in other systems (see, e.g., WO 03/020920). In one embodiment, undifferentiated pluripotent cells such as hES cells, can be cultured in a media comprising bFGF and TGFβ. Non-limiting example concentrations of bFGF include about 80 ng/ml. Non-limiting example concentrations of TGFβ include about 0.5 ng/ml. In yet another embodiment, undifferentiated pluripotent stem cells can be maintained in a commercially available, complete medium such as mTeSk^{rm} (Stem Cell Technologies, Vancouver, Canada).

Undifferentiated pluripotent cells can be cultured on a layer of feeder cells, typically fibroblasts derived from embryonic or fetal tissue (Thomson et al. (1998) Science 282:1145). Feeder cells can be derived from a human or a murine source. Human feeder cells can be isolated from various human tissues, or can be derived via differentiation of human embryonic stem cells into fibroblast cells (see, e.g., WO 01/51616). Human feeder cells that can be used include, but are not limited to, placental fibroblasts (see, e.g., Genbacev et al. (2005) Fertil. Steril. 83(5):1517), fallopian tube epithelial cells (see, e.g., Richards et al. (2002) Nat. Biotechnol., 20:933), foreskin fibroblasts (see, e.g., Amit et al. (2003) Biol. Reprod.68:2150), and uterine endometrial cells (see, e.g., Lee et al. (2005) Biol. Reprod. 72(1):42).

Various solid surfaces can be used in the culturing of undifferentiated pluripotent cells. Those solid surfaces include, but are not limited to, standard commercially available tissue culture flasks or cell culture plates, such as 6-well, 24-well, 96-well, or 144-well plates. Other solid surfaces include, but are not limited to, microcarriers and disks. Solid surfaces suitable for growing undifferentiated pluripotent cells can be made of a variety of substances including, but not limited to, glass or plastic such as polystyrene, polyvinylchloride, polycarbonate, polytetrafluorethylene, melinex, thermanox, or combinations thereof. Suitable surfaces can comprise one or more polymers, such as, e.g., one or more acrylates. A solid surface can be three-dimensional in shape. Non-limiting examples of three-dimensional solid surfaces have been previously described, e.g., in U.S. Patent Pub. No. 2005/0031598.

Undifferentiated stem cells can also be grown under feeder-free conditions on a growth substrate. A growth substrate can be a Matrigel® matrix (e.g., Matrigel®, Matrigel® GFR), recombinant laminin, laminin-511 recombinant fragment E8 or vitronectin. In certain embodiments of the present disclosure, the growth substrate is recombinant human laminin-521 (Biolamina, Sweden, distributed by Corning Inc., Corning, NY). In other embodiments, the substrate is a synthetic substrate, such as, for example, Synthemax®-II SC Substrate.

Undifferentiated stem cells can be passaged or subcultured using various methods such as using collagenase, or such as manual scraping. Undifferentiated stem cells can be subcultured by enzymatic means that generate a single cell suspension, such as using Accutase® (distributed by Sigma Aldrich, MO) or similar trypsinases. Alternatively, undifferentiated stem cells can be subcultured using non-enzymatic means, such as 0.5 mM EDTA in PBS, or such as using ReLeSR (Stem Cell Technologies, Vancouver, Canada).

In an embodiment, a plurality of undifferentiated stem cells are seeded or subcultured at a seeding density that allows the cells to reach confluence in about three to about ten days. In an embodiment, the seeding density can range from about 6.0×10³ cells/cm² to about 5.0×10⁵ cells/cm², such as about 1.0×10⁴ cells/cm², such as about 5.0×10⁴ cells/cm², such as about 1.0×10⁵ cells/cm², or such as about 3.0×10⁵ cells/cm² of growth surface. In another embodiment, the seeding density can range from about 6.0×10³ cells/cm² to about 1.0×10⁴ cells/cm² of growth surface, such as about 6.0 x 10 3 cells/cm² to about 9.0×10³ cells/cm², such as about 7.0×10³ cells/cm² to about 1.0×10⁴ cells/cm², such as about 7.0×10³ cells/cm² to about 9.0×10³ cells/cm², or such as about 7.0×10³ cells/cm² to about 8.0×10³ cells/cm² of growth surface. In yet another embodiment, the seeding density can range from about 1.0×10⁴ cells/cm² to about 1.0×10⁵ cells/cm² of growth surface, such as about 2.0×10⁴ cells/cm² to about 9.0×10⁴ cells/cm², such as about 3.0×10⁴ cells/cm² to about 8.0×10⁴ cells/cm², such as about 4.0×10⁴ cells/cm² to about 7.0×10⁴ cells/cm², or such as about 5.0×10⁴ cells/cm² to about 6.0×10⁴ cells/cm² of growth surface. In an embodiment, the seeding density can range from about 1.0×10⁵ cells/cm² to about 5.0×10⁵ cells/cm² of growth surface, such as about 1.0×10⁵ cells/cm² to about 4.5×10⁵ cells/cm², such as about 1.5×10⁵ cells/cm² to about 4.0×10⁵ cells/cm², such as about 2.0×10⁵ cells/cm² to about 3.5 x 10 5 cells/cm², or such as about 2.5×10⁵ cells/cm² to about 3.0×10⁵ cells/cm² of growth surface. The seeding density can be any value or subrange within the recited ranges.

Any of a variety of suitable cell culture and sub-culturing techniques can be used to culture stem cells in accordance with the methods of the present disclosure. For example, a culture medium can be completely exchanged daily, initiating about 2 days after sub-culturing of the cells. In an embodiment, when a culture reaches about 90% colony coverage, cells can be detached and seeded for subsequent culture using one or more suitable reagents, such as, e.g., Accutase® to achieve a single cell suspension for quantification. In an embodiment, undifferentiated stem cells can then be sub-cultured before seeding the cells on a suitable growth substrate (e.g., recombinant human laminin-521) at a seeding density that allows the cells to reach confluence over a suitable period of time, such as, e.g., in about three to ten days. In one embodiment, undifferentiated stem cells can be subcultured using Collagenase IV and expanded on a recombinant laminin In another embodiment, undifferentiated stem cells can be subcultured using Collagenase IV and expanded on a Matrigel®. In one embodiment, undifferentiated stem cells can be subcultured using ReLeSR TM and expanded on recombinant human laminin-521.

For seeding undifferentiated stem cells, the seeding density can range from about 6.0×10³ cells/cm² to about 5.0×10⁵ cells/cm², such as about 1.0×10⁴ cells/cm², such as about 5.0×10⁴ cells/cm², such as about 1.0×10⁵ cells/cm², or such as about 3.0×10⁵ cells/cm² of growth surface. In another embodiment, the seeding density can range from about 6.0×10³ cells/cm² to about 1.0×10⁴ cells/cm² of growth surface, such as about 6.0×10³ cells/cm² to about 9.0×10³ cells/cm², such as about 7.0×10³ cells/cm² to about 1.0×10⁴ cells/cm², such as about 7.0×10³ cells/cm² to about 9.0×10³ cells/cm², or such as about 7.0×10³ cells/cm² to about 8.0×10³ cells/cm² of growth surface. In yet another embodiment, the seeding density can range from about 1.0×10⁴ cells/cm² to about 1.0×10⁵ cells/cm² of growth surface, such as about 2.0×10⁴ cells/cm² to about 9.0×10⁴ cells/cm², such as about 3.0×10⁴ cells/cm² to about 8.0×10⁴ cells/cm², such as about 4.0×10⁴ cells/cm² to about 7.0×10⁴ cells/cm², or such as about 5.0×10⁴ cells/cm² to about 6.0×10⁴ cells/cm² of growth surface. In an embodiment, the seeding density can range from about 1.0×10⁵ cells/cm² to about 5.0×10⁵ cells/cm² of growth surface, such as about 1.0×10⁵ cells/cm² to about 4.5×10⁵ cells/cm², such as about 1.5×10⁵ cells/cm² to about 4.0×10⁵ cells/cm², such as about 2.0×10⁵ cells/cm² to about 3.5×10⁵ cells/cm², or such as about 2.5×10⁵ cells/cm² to about 3.0×10⁵ cells/cm² of growth surface. The seeding density can be any value or subrange within the recited ranges.

### V. Differentiation of Human Pluripotent Stem Cells to Dorsal Neuroectoderm Progenitor Cells and Further to Dorsally-Derived Glial Progenitor Cells and Oligodendrocyte Progenitor Cells

The present disclosure provides methods for differentiating human pluripotent stem cells into neuroectoderm with a dorsal spinal cord phenotype and further to glial progenitor cells and oligodendrocyte progenitor cells using a combination of small molecule and protein modulators of BMP signaling and inhibitors of MAPK/ERK kinase. Without being held to any particular theory, the inventors have discovered that human dorsal neuroectoderm progenitor cells obtained in accordance with methods of the present disclosure can be readily and efficiently differentiated into spinal cord OPCs in the absence of activation of SHH signaling. Surprisingly, this early dorsal phenotype, despite not being the region of early OPC generation in vivo, gives rise to glial progenitor cells by day 21 of the differentiation process and to OPCs by day 28 of the differentiation process. The day 28 OPCs produced in accordance with the present disclosure express canonical OPC markers NG2 and PDGFRa and are comparable (in terms of their overall marker expression profile) to OPCs generated using an alternative method that are currently in clinical testing to treat spinal cord injury.

In an embodiment, a method comprises contacting human pluripotent stem cells with one or more inhibitors of mitogen-activated protein kinase/extracellular signal regulated kinase (MAPK/ERK) combined with one or more inhibitors of bone morphogenic protein (BMP) signaling. In certain embodiments, the MAPK/ERK inhibitor is a small molecule. In other embodiments, the MAPK/ERK inhibitor is a protein, such as a phosphatase that dephosphorylates the MAPK/ERK kinase. In certain embodiments, the inhibitor of BMP signaling is a small molecule. In other embodiments, the inhibitor of BMP signaling is a protein. In some embodiments, the direct target of the inhibitor of BMP signaling is ALK2, also known as Activin A receptor, type I (ACVR1). In certain embodiments, subsequent to MAPK/ERK and BMP signaling inhibition, the cells are cultured in the presence of caudalizing agent retinoic acid, thereby obtaining neuroectoderm-restricted progenitor cells with a dorsal spinal cord phenotype.

In certain embodiments, an inhibitor of MAPK/ERK can be selected from the group consisting of PD0325901, AZD6244, GSK1120212, PD1 84352 and Cobimetinib, and derivatives thereof. In certain embodiments, an inhibitor of BMP signaling can be selected from the group consisting of Dorsomorphin, DMH-1, K02288, ML347, LDN193189 and Noggin protein.

In an embodiment, a method comprises obtaining undifferentiated human pluripotent stem cells that remain in undifferentiated state; culturing the undifferentiated human pluripotent stem cells adherently in the presence of small molecules PD0325901 and Dorsomorphin and retinoic acid for a first time period; and subsequently culturing the cells adherently in the presence of retinoic acid and in the absence of a SHH signaling activator for a second time period, thereby obtaining dorsal neuroectoderm cells. In an embodiment, the first time period and the second time period can each range from about one to about six days, such as about one day, such as about two days, such as about three days, such as about four days, such as about five days, or such as about six days.

In an embodiment, a method comprises culturing undifferentiated human pluripotent stem cells in the presence of PD0325901 at a concentration that ranges from about 1 μM to about 100 μM, such as about 2 μM, such as about 3 μM, such as about 4 μM, such as about 5 μM, such as about 6 μNI, such as about 7 μM, such as about 8 μM, such as about 9 μM, such as about 10 μM, such as about 11 μM, such as about 12 μM, such as about 13 μM, such as about 14 μM, such as about 15 μM, such as about 20 μM, such as about 30 μM, such as about 40 μM, such as about 50 μM, or such as about 60 μM, 70 μM, 80 μM or 90 μM. In another embodiment, a method comprises culturing undifferentiated human pluripotent stem cells in the presence of PD0325901 at a concentration of about 10 μM. The concentration can be any value or subrange within the recited ranges.

In an embodiment, a method comprises culturing undifferentiated human pluripotent stem cells in the presence of AZD6244 at a concentration that ranges from about 1 μM to about 100 μM, such as about 2 μM, such as about 3 μM, such as about 4 μM, such as about 5 μM, such as about 6 μM, such as about 7 μM, such as about 8 μM, such as about 9 μM, such as about 10 μM, such as about 11 μM, such as about 12 μM, such as about 13 μM, such as about 14 μM, such as about 15 μM, such as about 20 μM, such as about 30 μM, such as about 40 μM, such as about 50 μM, or such as about 60 μM, 70 μM, 80 μM or 90 μM. In another embodiment, a method comprises culturing undifferentiated human pluripotent stem cells in the presence of AZD6244 at a concentration of about 10 μM. The concentration can be any value or subrange within the recited ranges.

In an embodiment, a method comprises culturing undifferentiated human pluripotent stem cells in the presence of GSK1120212 at a concentration that ranges from about 1 μM to about 100 μM, such as about 2 μM, such as about 3 μM, such as about 4 μM, such as about 5 μM, such as about 6 μM, such as about 7 μM, such as about 8 μM, such as about 9 μM, such as about 10 μM, such as about 11 μM, such as about 12 μM, such as about 13 μM, such as about 14 μM, such as about 15 such as about 20 μM, such as about 30 μM, such as about 40 μM, such as about 50 μM, or such as about 60 μM, 70 μM, 80 μM or 90 μM of PD0325901. In another embodiment, a method comprises culturing undifferentiated human pluripotent stem cells in the presence of GSK1120212 at a concentration of about 10 μM. The concentration can be any value or subrange within the recited ranges.

In an embodiment, a method comprises culturing undifferentiated human pluripotent stem cells in the presence of PD184352 at a concentration that ranges from about 1 μM to about 100 μM, such as about 2 μM, such as about 3 μM, such as about 4 μM, such as about 5 μM, such as about 6 μM, such as about 7 μM, such as about 8 μM, such as about 9 μM, such as about 10 μM, such as about 11 μM, such as about 12 μM, such as about 13 μM, such as about 14 μM, such as about 15 μM, such as about 20 μM, such as about 30 μM, such as about 40 μM, such as about 50 μM, or such as about 60 μM, 70 μM, 80 μM or 90 μM. In another embodiment, a method comprises culturing undifferentiated human pluripotent stem cells in the presence of PD184352 at a concentration of about 10 μM. The concentration can be any value or subrange within the recited ranges.

In an embodiment, a method comprises culturing undifferentiated human pluripotent stem cells in the presence of Cobimetinib at a concentration that ranges from about 1 μM to about 100 μM, such as about 2 μM, such as about 3 μM, such as about 4 μM, such as about 5μM, such as about 6 μM, such as about 7 μM, such as about 8 μM, such as about 9 μM, such as about 10 μM, such as about 11 μM, such as about 12 μM, such as about 13 μM, such as about 14 μM, such as about 15 μM, such as about 20 μM, such as about 30 μM, such as about 40 μM, such as about 50 μM, or such as about 60 μM, 70 μM, 80 μM or 90 μM. In another embodiment, a method comprises culturing undifferentiated human pluripotent stem cells in the presence of Cobimetinib at a concentration of about 10 μM. The concentration can be any value or subrange within the recited ranges.

In an embodiment, a method comprises culturing undifferentiated human pluripotent stem cells in the presence of Dorsomorphin at a concentration that ranges from about 0.2 μM to about 20 μM, such as about 0.5 μM, such as about 0.8 μM, such as about 1 μM, such as about 1.5 μM, such as about 2 μM, such as about 2.5 μM, such as about 3 μM, such as about 3.5 μM, such as about 4 μM, such as about 4.5 μM, such as about 5 μM, such as about 5.5 μM, such as about 6 μM, such as about 6.5 μM, such as about 7 μM, such as about 7.5 μM, such as about 8 μM, such as about 8.5 μM, such as about 9 μM, such as about 10 μM, such as about 11 μM, such as about 12 μM, such as about 13 μM, such as about 14 μM, such as about 15 μM, such as about 16 μM, such as about 17 μM, such as about 18 μM, or such as about 19 μM. In another embodiment, a method comprises culturing undifferentiated human pluripotent stem cells in the presence of Dorsomorphin at a concentration that ranges from about 0.2 μM to about 1 μM, such as about 0.2 μM to about 0.9 μM, such as about 0.3 μM to about 0.8 μM, such as about 0.4 μM to about 0.7 μM, or such as about 0.5 μM to about 0.6 μM. In yet another embodiment, a method comprises culturing undifferentiated human pluripotent stem cells in the presence of Dorsomorphin at a concentration that ranges from about 1 μM to about 10 μM, such as about 1 μM to about 9 μM, such as about 2 μM to about 8 μM, such as about 3 μM to about 7 μM, or such as about 4 μM to about 6 μM. In an embodiment, a method comprises culturing undifferentiated human pluripotent stem cells in the presence of Dorsomorphin at a concentration that ranges from about 10 μM to about 20 μM, such as about 10 μM to about 19 μM, such as about 12 μM to about 18 μM, such as about 13 μM to about 17 μM, or such as about 14 μM to about 16 μM. In an embodiment, a method comprises culturing undifferentiated human pluripotent stem cells in the presence of Dorsomorphin at a concentration of about 2 μM. The concentration can be any value or subrange within the recited ranges.

In an embodiment, a method comprises culturing undifferentiated human pluripotent stem cells in the presence of an ALK2 inhibitor at a concentration that ranges from about 1 nM to about 20 μM, such as about 10 nM, about 50 nM, about 100 nM, about 150 nM, about 200 nM, about 500 nM, about 1 μM, about 5 μM, about 10 μM, or about 15 μM. The concentration can be any value or subrange within the recited ranges.

In an embodiment, a method comprises culturing undifferentiated human pluripotent stem cells in the presence of DMH-1 at a concentration that ranges from about 1 μM to about 10 μM. In an embodiment, a method comprises culturing undifferentiated human pluripotent stem cells in the presence of DMH-1 at about 2 μM. The concentration can be any value or subrange within the recited ranges.

In an embodiment, a method comprises culturing undifferentiated human pluripotent stem cells in the presence of K02288 at a concentration that ranges from about 1 μM to about 10 μM. In an embodiment, a method comprises culturing undifferentiated human pluripotent stem cells in the presence of K02288 at about 2 μM. The concentration can be any value or subrange within the recited ranges.

In an embodiment, a method comprises culturing undifferentiated human pluripotent stem cells in the presence of ML347 at a concentration that ranges from about 1 μM to about 10 μM. In an embodiment, a method comprises culturing undifferentiated human pluripotent stem cells in the presence of ML347 at about 2 μM. The concentration can be any value or subrange within the recited ranges.

In an embodiment, a method comprises culturing undifferentiated human pluripotent stem cells in the presence of LDN193189 at a concentration that ranges from about 1 μM to about 10 μM. In an embodiment, a method comprises culturing undifferentiated human pluripotent stem cells in the presence of LDN193189 at about 2 μM. The concentration can be any value or subrange within the recited ranges.

Any tissue culture vessels suitable for adherent cell culture may be used for obtaining dorsal neuroectoderm progenitor cells in accordance with the present disclosure. Suitable growth substrates include, for example, recombinant laminin, laminin-511 recombinant fragment E8 or a Matrigel® matrix (e.g., Matrigel®, Matrigel® GFR), dorsomorphin, PD0325901, retinoic acid (RA), ascorbic acid (AA), recombinant human EGF (rhEGF), heat stable (recombinant human basic FGF (rhbFGF), ROCK inhibitor, rhLN521or PDGF-A.

In an embodiment, the dorsal neuroectoderm progenitor cells obtained in accordance with the present disclosure can be harvested and cultured further as aggregates in suspension culture in the presence of heat stable rhbFGF and EGF until the cells have matured into glial progenitor cells. In an embodiment, the further culturing period can range from about five to fifteen days, such as about five days, about six days, about seven days, about eight days, about nine days, about ten days, about eleven days, about twelve days, about thirteen days, about fourteen days, or about fifteen days. In an embodiment, the further culturing period is about seven days.

In an embodiment, adherently cultured dorsal neuroectoderm progenitor cells can be harvested by enzymatic means. Enzymatic means include, without limitation, TrypLE™ Select (Thermo Fisher Scientific, Waltham, MA), Accutase® (Sigma Aldrich, MO) or similar trypsinases. Alternatively, adherently cultured dorsal neuroectoderm progenitor cells can be harvested using non-enzymatic means. Non-enzymatic means include, without limitation, 0.5 mM EDTA in PBS, or such as using ReLeSR™ (Stem Cell Technologies, Vancouver, Canada).

Any cell culture vessels or reactors suitable for suspension culture can be used for the non-adherent culture steps contemplated in the present disclosure. The vessel walls are typically inert or resistant to adherence of the cultured cells. For dynamic suspension, there is also a means for preventing the cells from settling out, such as a stirring mechanism like a magnetically or mechanically driven stir bar or paddle, a shaking mechanism (typically attached to the vessel by the outside), or an inverting mechanism (i.e., a device that rotates the vessel so as to change the direction of gravity upon the cells).

Vessels suitable for suspension culture for process development include the usual range of commercially available spinner, spinner flasks, rocker bag, or shaker flasks. Example bioreactors suitable for commercial production include the VerticalWheel TM Bioreactors (PBS Biotech, Camarillo, CA).

Aggregates can also be formed prior to dynamic suspension. For example, the cells can be placed on AggreWell™ plates to generate uniform cell aggregates. After about three days, the cell aggregates can be transferred to dynamic suspension.

In an embodiment, glial progenitor cells obtained in accordance with the present disclosure can be harvested, plated down and cultured adherently for a further time period in the presence of epidermal growth factor (EGF) until the cells have matured into oligodendrocyte progenitor cells. In certain embodiments, the culture medium additionally comprises platelet-derived growth factor AA (PDGF-AA). In an embodiment, the further culturing period can range from about from about seven to fourteen days, such as about seven days, about ten days, about twelve days, or about fourteen days. In an embodiment, the further culturing period ranges from about fourteen to twenty-five days, such as about fifteen days, about sixteen days, about seventeen days, about nineteen days, about twenty days, about twenty-one days, about twenty-two days, about twenty-three days, about twenty-four days, or about twenty-five days. The culturing period may be any value or subrange within the recited ranges, including endpoints.

### VI. Undesirable Cell Types

OPC populations obtained according to the present disclosure contain low levels of undesired ell types, as measured, for example, by quantification of markers associated with undesirable cell types by flow cytometry. In a non-limiting example, the Day 35 OPCs obtained according to the present disclosure may contain low levels of cells expressing the epithelial cell associated markers cytokeratin 7 (CK7), EpCAM, CD49f, and CLDN6.In another non-limiting example, the Day 42 OPCs obtained according to the present disclosure may contain low levels of cells expressing the epithelial cell associated markers ck7, EpCAM, CD49f, and CLDN6 with no hESCs residuals associated with the markers TRA-1-60 and OCT4.

Markers associated with undesirable cell types can comprise less than about 20% undesirable cell types, such as less than about 19%, such as less than about 18%, such as less than about 17%, such as less than about 16%, such as less than about 15%, such as less than about 14%, such as less than about 13%, such as less than about 12%, such as less than about 11%, such as less than about 10%, such as less than about 9%, such as less than about 8%, such as less than about 7%, such as less than about 6%, such as less than about 5%, such as less than about 4%, such as less than about 3%, such as less than about 2%, such as less than about 1%, such as less than about 0.5%, such as less than about 0.1%, such as less than about 0.05%, or such as less than about 0.01% undesirable cell types. In another embodiment, a cell population can comprise from about 15% to about 20% undesirable cell types, such as about 19% to about 20%, such as about 18% to about 20%, such as about 17% to about 20%, such as about 16% to about 20%, such as about 15% to about 19%, or such as about 16% to about 18% undesirable cell types. In yet another embodiment, a cell population can comprise from about 10% to about 15% undesirable cell types, such as about 14% to about 15%, such as about 13% to about 15%, such as about 12% to about 15%, such as about 11% to about 15%, or such as about 12% to about 14% undesirable cell types. In an embodiment, a cell population can comprise from about 1% to about 10% undesirable cell types, such as about 2% to about 10%, such as about 1% to about 9%, such as about 2% to about 8%, such as about 3% to about 7%, or such as about 4% to about 6% undesirable cell types. In an embodiment, a cell population can comprise from about 0.1% to about 1% undesirable cell types, such as about 0.2% to about 1%, such as about 0.1% to about 0.9%, such as about 0.2% to about 0.8%, such as about 0.3% to about 0.7%, or such as about 0.4% to about 0.6% undesirable cell types. In an embodiment, a cell population can comprise from about 0.01% to about 0.1% undesirable cell types, such as about 0.02% to about 0.1%, such as about 0.01% to about 0.09%, such as about 0.02% to about 0.08%, such as about 0.03% to about 0.07%, or such as about 0.04% to about 0.06% undesirable cell types. In an embodiment, low levels of undesirable cell types can denote the presence of less than about 15% undesirable cell types.

In an embodiment, an undesirable cell type can comprise cells expressing one or more markers selected from ck7, CD49f, CLDN6, or EpCAM.

### VII. Cryopreservation

Following harvesting, the expanded population of OPCs can be formulated at a specific therapeutic dose (e.g., number of cells) and cryopreserved for shipping to the clinic. The ready to administer (RTA) OPC therapy composition can then be administered directly after thawing without further processing. Examples of media suitable for cryopreservation include but are not limited to 90% Human Serum/10% DMSO, Media 3 10% (CS10), Media 2 5% (CS5) and Media 1 2% (CS2), Stem Cell Banker, PRIME XV® FREEZIS, HYPOTHERMASOL®, CSB, Trehalose, etc.

In some embodiments, the percent viability of post-filtered cells stored in a cryopreservation medium for between about 0 to about 8 hours is at least about 75%, 80%, 85%, 90%, 91%, 92%, 93%, 94%, 95%, 96%, 97%, 98%, 99%, or 100%. In other embodiments, the percent recovery of post-filtered cells stored in a cryopreservation medium for between about 0 to about 8 hours is at least about 75%, 80%, 85%, 90%, 91%, 92%, 93%, 94%, 95%, 96%, 97%, 98%, 99%, or 100%. The viability can be any value or subrange within the recited ranges.

In further embodiments, the percent viability of post-filtered cells stored in a neutralization medium for between about 0 to about 8 hours followed by storage in cryopreservation medium for between about 0 to about 8 hours is at least about 75%, 80%, 85%, 90%, 91%, 92%, 93%, 94%, 95%, 96%, 97%, 98%, 99%, or 100%. In other embodiments, the percent recovery of post-filtered cells stored in a neutralization medium for between about 0 to about 8 hours followed by storage in cryopreservation medium for between about 0 to about 8 hours is at least about 70%, 75%, 80%, 85%, 90%, 91%, 92%, 93%, 94%, 95%, 96%, 97%, 98%, 99%, or 100%. The viability can be any value or subrange within the recited ranges.

In yet other embodiments, the percent viability of post-filtered cells stored in a neutralization medium for between about 0 to about 8 hours followed by storage in cryopreservation medium for between about 0 to about 8 hours, post-thawing of the cryopreserved composition, is at least about 75%, 80%, 85%, 90%, 91%, 92%, 93%, 94%, 95%, 96%, 97%, 98%, 99%, or 100%. In still other embodiments, the percent recovery of post-filtered cells stored in a neutralization medium for between about 0 to about 8 hours followed by storage in cryopreservation medium for between about 0 to about 8 hours, post-thawing of the cryopreserved composition, is at least about, 70%, 75%, 80%, 85%, 90%, 91%, 92%, 93%, 94%, 95%, 96%, 97%, 98%, 99%, or 100%. The viability can be any value or subrange within the recited ranges.

In some embodiments, post-filtered OPCs stored in a neutralization medium for between about 0 to about 8 hours followed by storage in cryopreservation medium for between about 0 to about 8 hours, post-thawing of the cryopreserved composition are capable of secreting Decorin. In other embodiments, post-filtered OPCs stored in a neutralization medium for between about 0 to about 8 hours followed by storage in cryopreservation medium for between about 0 to about 8 hours, post-thawing of the cryopreserved composition are capable of being expanded.

In some embodiments, the percent viability of post-filtered OPCs stored in a neutralization medium for between about 0 to about 8 hours at room temperature is at least about, 70%, 75%, 80%, 85%, 90%, 91%, 92%, 93%, 94%, 95%, 96%, 97%, 98%, 99%, or 100%. In some embodiments, the percent viability of post-filtered OPCs stored in a cryopreservation medium for between about 0 to about 8 hours at room temperature is at least about, 70%, 75%, 80%, 85%, 90%, 91%, 92%, 93%, 94%, 95%, 96%, 97%, 98%, 99%, or 100%. In further embodiments, the percent viability of post-filtered cells stored in a neutralization solution at room temperature for between about 0 to about 8 hours followed by storage in cryopreservation medium for between about 0 to about 8 hours at room temperature is at least about 75%, 80%, 85%, 90%, 91%, 92%, 93%, 94%, 95%, 96%, 97%, 98%, 99%, or 100%. In still further embodiments, the percent recovery of post-filtered cells stored in a neutralization solution at room temperature for between about 0 to about 8 hours followed by storage in cryopreservation medium for between about 0 to about 8 hours at room temperature is at least about 75%, 80%, 85%, 90%, 91%, 92%, 93%, 94%, 95%, 96%, 97%, 98%, 99%, 100%, 105%, 110%, 115%, 120%, 125%, 130%, 140%, 150%. The viability can be any value or subrange within the recited ranges.

OPCs formulated in cryopreservation media appropriate for post thaw ready to administer (RTA) applications may comprise OPCs suspended in adenosine, dextran-40, lactobionic acid, HEPES (N-(2-Hydroxyethyl) piperazine-N′-(2-ethanesulfonic acid)), sodium hydroxide, L-glutathione, potassium chloride, potassium bicarbonate, potassium phosphate, dextrose, sucrose, mannitol, calcium chloride, magnesium chloride, potassium hydroxide, sodium hydroxide, dimethyl sulfoxide (DMSO), and water. An example of this cryopreservation media is available commercially under the tradename, CRYOSTOR® and is manufactured by BioLife Solutions, Inc.

DMSO can be used as a cryoprotective agent to prevent the formation of ice crystals, which can kill cells during the cryopreservation process. In some embodiments, the cryopreservable OPC therapy composition comprises between about 0.1% and about 2% DMSO (v/v). In some embodiments, the RTA OPC therapy composition comprises between about 1% and about 20% DMSO. In some embodiments, the RTA OPC therapy composition comprises about 10% DMSO. In some embodiments, the RTA OPC cell therapy composition comprises about 5% DMSO. The concentration can be any value or subrange within the recited ranges.

In some embodiments, OPC therapies formulated in cryopreservation media appropriate for post thaw ready to administer (RTA) applications may comprise OPCs suspended in cryopreservation media that does not contain DMSO. For example, RTA OPC therapeutic cell compositions may comprise OPCs suspended in Trolox, Na+, K+, Ca2+, Mg2+, cl−, H2P04—, HEPES, lactobionate, sucrose, mannitol, glucose, dextran-40, adenosine, glutathione without DMSO (dimethyl sulfoxide, (CH3)2S0) or any other dipolar aprotic solvents. An example of this cryopreservation media is available commercially under the tradename, HYPOTHERMOSOL® or HYPOTHERMOSOC-FRS and is also manufactured by BioLife Solutions, Inc. In other embodiments, OPC compositions formulated in cryopreservation media appropriate for post thaw ready to administer applications may comprise OPCs suspended in Trehalose.

The RTA OPC therapy composition may optionally comprise additional factors that support OPC engraftment, integration, survival, potency, etc. In some embodiments, the RTA OPC therapy composition comprises activators of function of the OPC preparations described herein.

In some embodiments, the RTA OPC therapy composition may be formulated in a medium comprising components that decrease the molecular cell stress during freezing and thawing processes by scavenging of free radicals, pH buffering, oncotic/osmotic support and maintenance of the ionic concentration balance.

In some embodiments, OPC therapies formulated in cryopreservation media appropriate for post thaw ready to administer applications may comprise one or more immunosuppressive compounds. In certain embodiments, OPC therapies formulated in cryopreservation media appropriate for post thaw ready to administer applications may comprise one or more immunosuppressive compounds that are formulated for slow release of the one or more immunosuppressive compounds. Immunosuppressive compounds for use with the formulations described herein may belong to the following classes of immunosuppressive drugs: Glucocorticoids, Cytostatics (e.g. alkylating agent or antimetabolite), antibodies (polyclonal or monoclonal), drugs acting on immunophilins (e.g. cyclosporin, Tacrolimus or Sirolimus). Additional drugs include interferons, opioids, TNF binding proteins, mycophenolate and small biological agents. Examples of immunosuppressive drugs include: mesenchymal stem cells, anti-lymphocyte globulin (ALG) polyclonal antibody, anti-thymocyte globulin (ATG) polyclonal antibody, azathioprine, BAS 1L1×1MABO (anti-IL-2Ra receptor antibody), cyclosporin (cyclosporin A), DACLIZUMAB® (anti-I L-2Ra receptor antibody), everolimus, mycophenolic acid, RITUX1MABO (anti-CD20 antibody), sirolimus, tacrolimus, Tacrolimus and or Mycophenolate mofetil.

Having now generally described the invention, the same will be more readily understood through reference to the following examples that are provided by way of illustration, and are not intended to be limiting of the present disclosure, unless specified. It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

### Example 1: Establishment of OPC1 Thaw-and-Inject (TAI) Drug Product (DP) Formulation

The purpose of this example is to demonstrate establishment of a new oligodendrocyte progenitor cell (OPC1) thaw-and-inject (TAI) drug product formulation. OPC1 Drug Product (DP) for TAI was developed to be thawed and directly applied at the clinical sites. Previously, a series of studies were conducted to develop an improved scaled-up production and harvesting process of OPC1 cells. The focus of this Example is establishing the TAI DP formulation using CryoStor 10 (CS10) as the cryopreservation media for generating clinical dosages of 100×10⁶ live cells per ml with a final volume of 30010 cell suspension per vial, as well as a method for thawing OPC1 TAI vials.

The following aspects are specifically established in this Example; establishment of OPC1 TAI formulation as 100×10⁶ live cells per ml. in CS10, with a final volume of 300 μl cell suspension per vial; establishment of the thawing procedure of OPC1 TAI vials containing 300 μl; evaluation of the comparability of OPC1 TAI obtained from LN521-expanded cells vs. iMatrix511-E8-expanded cells; evaluation of the optimal cell dissociating enzyme neutralization solution; and corroborating the ability of the optimized scaled-up OPC differentiation process to generate OPC1 TAI meeting all acceptance criteria.

The following abbreviations and definitions are used in this Example. CF—Cell Factory) cell culturing vessel). CS10—CryoStor 10 (cell cryopreservation agent). DMEM/F12—cell culture media. DP—Drug Product. DS—Drug Substance. GF—Growth Factors. HSA—Human Serum Albumin ICB—Intermediate Cell Bank. iMatrix511-E8—Fragmented laminin-511 coating matrix. LN521—Laminin-521 coating matrix. Min.—Minute(s). NUTS (−) with HSA—NutriStem (cell culture media) containing Human Serum Albumin. OPC1 TAI—OPC1® ‘Thaw-and-Inject’ formula. RT—Room Temperature. Sec.—Second(s). TS—TrypLE Select (cell dissociating enzyme).

Tables 1 and 2 below list the materials and equipment used in this Example.

TABLE 1
Study Materials

| Material | Manufacturer | Cat. No. |
|---|---|---|
| DMEM/F-12 GlutMax | Gibco | 10565-018 |
| Via-2 Cassette | Chemometec | 941-0024 |
| DPBS (−) | HyClone | SH30028 |
| DPBS (+) | HyClone | SH30264.02 |
| iMatrix511−E8 | nippi | 892012 |
| LN521 | Bio Lamina | LN521-05/LN521-01 |
| NUNC Cell Factory | Thermo Scientific | 140000/165250 |
| NUT (−) with HSA | Biological Industries | 06-5100-01-1B |
| TrypLE Select | Gibco | 12563-011/029 |

TABLE 2
Equipment

| Equipment | Manufacturer |
|---|---|
| Centrifuge 5810R | Eppendorf |
| Centrifuge 5804 | Eppendorf |
| 8-channel Decapper | Thermo Scientific |
| Multipipette E3 | Eppendorf |
| CryoMed | Thermo Scientific |
| CryoMed | Thermo Scientific |
| NC-200 Cell Counter | Chemometec |

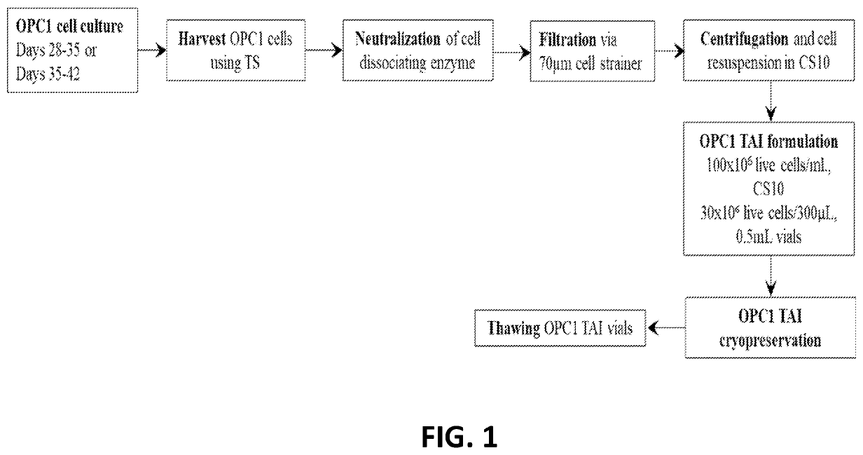

FIG. 1 presents an overview of the experimental procedure for OPC1 TAI harvesting and cryopreservation. As shown, OPC1 cells are cultured until days 28-35 or days 35-42. Next, OPC1 cells are harvested using TS, followed by neutralization of cell dissociating enzyme, filtration via 70 um cell strainer, and centrifugation and cell resuspension in CS10. OPC1 TAI formulation is concentrated at 100×10⁶ live cells per mL in CS10 and stored at a concentration of 30×10⁶ live cells per 300 uL in 0.5 mL vials. Finally, OPC1 TAI cryopreservation is performed, and ultimately OPC1 TAI vials are thawed.

Table 3 below lists details and objectives for several TAI experiments.

TABLE 3
Test Conditions

| | | | HESCs | Vessel | Enzyme | | |
|---|---|---|---|---|---|---|---|
| Experiment | Vessel | Harvesting | media in | coating | neutralization | | |
| # | type | Day | Stage I | agent | solution | Test objective(s) | |
| TAI#3 | T225 | 42 | mTeSR1 | LN521 | GPM+ | Establish the TAI formulation | |
| | | | | | | technique and generate TAI vials. | |
| TAI#4 | T225 | 42 | mTeSR1 | LN521 | GPM+ | Establish the TAI formulation | |
| | | | | | | technique and generate TAI vials. | |
| TAI#5 | T75 | 42 | mTeSR | LN521 | GPM+ | Establish the TAI formulation | |
| | | | Plus | | | technique and generate TAI vials. | |
| | | | | | | Assess TAI cell expansion post | |
| | | | | | | day 42 (morphology only). | |
| TAI#6 | T225 | 42 | mTeSR | LN521 | (A) GPM+ | 1. | Evaluate several solutions for |
| | | | Plus | | (B) DMEM/F12 | | neutralization of TS cell |
| | | 42 | mTeSR | | (C) NUT (−) with | | dissociating enzyme pre final |
| | | | Plus | | HSA | | formulation and cryopreservation |
| | | | | | | | on Day 42. |
| | | | | | | 2. | Elimination of animal-derived |
| | | | | | | | components from the TS |
| | | | | | | | neutralization solution. |
| TAI#7 | T225 | 35 | mTeSR | LN521 | GPM+ | Generate TAI vials from an | |
| | | | Plus | | | ongoing culture on Day 35 to | |
| | | | | | | evaluate feasibility of earlier cell | |
| | | | | | | harvesting day. | |
| TAI#8 | 1-layer | 42 | mTeSR | LN521 | GPM+ | 1^{st} evaluation of Day 42 cells | |
| | CF | | Plus | | | harvested from 1-layer LN521- | |
| | | | | | | coated CF. | |
| TAI#11 | 1-layer | 42 | mTeSR | iMatrix511- | GPM+ | Evaluation of Day 42 cells | |
| | CF | | Plus | E8 | | harvested from 1-layer Cell | |
| | | | | | | Factory, originating from an | |
| | | | | | | ongoing ICB14#1 culture. | |
| TAI#12 | 1-layer | 42 | mTeSR | iMatrix511- | (A) GPM+ | 1. | Evaluation of Day 42 cells |
| | CF | | Plus | E8 | (B) DMEM/F12 with | | harvested from iMatrix-511 E8 |
| | | | | | 1% HSA² | | direct-coated 1-layer CF. |
| | | | | | (C) NUT (−) with | 2. | Re-evaluation of 3 types of TS |
| | | | | | HSA | | neutralization solution. |

| | | | | | | |
|---|---|---|---|---|---|---|
| TAI#13 | 1-layer | 35 | mTeSR | LN521 | NUT (−) with | 2^{nd} evaluation of Day 35 cells |
| | CF | | Plus | | HSA | harvested from LN521-coated CF. |
| DEVOPC_Test#25 | 1-layer | 42 | mTeSR | iMatrix511- | NUT (−) with | Assess the effect of filtration using |
| | CF | | Plus | E8 | HSA | a 60 μm filter on TAI DP in the |
| | | | | | | complete established harvesting |
| | | | | | | procedure. |

²1% HSA based on previous administration of GPOR Dose Preparation of 1% HSA in Isolyte S

In Table 3 above, experiments TAI #9 and TAI #10 are not included as the 2-layer CF was found to be unsupportive of OPC1 expansion.

### OPC1 Culturing (Stage II)

OPC1 cells were cultured in CF (either direct-coated with iMatrix511-E8 or pre-coated with LN521) for 7 days. Initial qualification of the final DP's volume and cell concentration was executed using OPC1 cells originating in H1 cells cultured using mTeSR1 (TAI #3 and #4) or mTeSR Plus medium (TAI #5-13), in LN521-coated vessels and differentiated with GPM (+) as culture media. TAI #11 and TAI #12 later established DP formulation from cells cultured when applying the direct-coating method using iMatrix511-E8-supplemented seeding media rather than LN521-coated flasks.

### Harvesting OPC

Cells were harvested on day 35 or day 42 using TS. Three cell dissociating enzyme (TS) neutralization solutions were tested (TAI #6 and #12) to evaluate their effect on TAI cell quality attributes and upgrading raw materials' GMP grade: GPM+(without GF) which was the originally established solution for OPC culturing, DMEM/F12—as is or supplemented with 1% HSA—and NUT (−) with HSA. The selected cell dissociating enzyme (TS) neutralization solution, NUT (−) with HSA, was applied in studies TAI #13 and DEVOPC_Test #25.

### Filtration and Centrifugation of OPC1 DS

OPC1 TAI is intended to be administered as a single-cell suspension. Obtaining a single-cell DS required that the DS obtained from cell harvesting be passed via a 70 μm cell strainer. Following filtration, the cell suspension was centrifuged (sup discarded) and continued immediately to the final formulation of the DP.

### OPC1 TAI DP Formulation

Cell pellets obtained following DS centrifugation were resuspended in CS10 for generating a final DP cell concentration of 100×10⁶ live cells/ml.

### DP Aliquoting Into Vials

DP following final formulation is immediately aliquoted into vials; 30×10⁶ live cells per 300 μl, per vial—using automated dispenser and capper/decapper—which then continue to cryopreservation.

### Thawing OPC1 TAI Vials

The need for thawing the TAI vials generated during the course of this study for cell analyses mandated conducting a comparability test which evaluated a dry-bath based thawing method using the MD-Mini dry bath with a customized heating block for accommodating the vial, compared to the standard thawing method of using a 37° C. water bath.

### QC Assays

Thawed TAI vials were assayed for biomarkers expression, on Day 35 or Day 42. Thawed cells were assayed for Decorin secretion.

### Acceptance Criteria

Purity markers (PDGFRα)>95.00%. Impurities markers (Claudin 6, EpCAM, CK7) <2.00%. Decorin secretion (day 42 cells)≥15 ng/ml. Morphology: small and compact cells, absence of elongated spindle-like morphology.

### Recovery Calculations

Recovery percentages were calculated based on the targeted final DP concentration of 100×10⁶ live cells/ml.

### Results

Establishing a thawing method for OPC1 TAI vials. OPC1 TAI was developed to be thawed and directly applied at the clinical sites hence the requirement for establishing a robust, portable and simple method for thawing vials cryopreserved and stored in vapor-phase LN2. This mandated conducting a comparability test which evaluated a dry-bath based thawing method using the MD-Mini dry bath with a customized heating block for accommodating the vial, compared to the common thawing method using a 37° C. water bath. Preliminary studies using 0.5 ml cryovials filled with 300 μl CS10 to simulate actual volume of the clinical vials established the optimal thawing duration in the MD-Mini dry-bath as 2 minutes, and 1 min25sec in the 37° C. water bath. Additionally, it was established (TAI #3/4) that thawed cells should be incubated in GPM+ for 2 minutes before assayed for % Recovery and % Viability using the NC-200 cell counter. Thereafter, all TAI vials were thawed in one of the aforementioned methods, both for R&D and QC analyses.

Establishing the OPC1 TAI DP cell concentration and volume

OPC 1 TAI DP was established as a 300 μl CS 10 cell suspension with a concentration of 100×10⁶ live cells/mi., for a total of 30×10⁶ live cells per vial. The following Table 4 summarizes the post-thawing % Recovery and % Viability values of TAI vials of the relevant studies.

TABLE 4
TAI post-thawing % Recovery and % Viability results

| | | Harvest | Avg. % Viability | | Avg. % Recovery | |
|---|---|---|---|---|---|---|
| Test #/(group) | | Day | Vial #1 | Vial #2 | Vial #1 | Vial #2 |
| TAI#3 (2 min.) | | 42 | 96 | NA | 117 | NA |
| TAI#4 (2 min.) | | 42 | 97 | NA | 98 | NA |
| TAI#5 | | 42 | 95 | NA | 95 | NA |
| TAI#6 | (A) GPM+ | 42 | 96 | NA | 90 | NA |
| | (B) DMEM/F12 | | 86 | 87 | 90 | 110 |
| | (C) NUT (−) | | 94 | 94 | 97 | 100 |
| | with HSA | | | | | |
| TAI#7 | | 35 | 92 | NA | 94 | NA |
| TAI#8 | | 42 | 98 | 97 | 104 | 106 |
| TAI#11 | | 42 | 88 | NA | 100 | NA |
| TAI#12 | (A) GPM+ | 42 | 97 | 95 | 97 | 95 |
| | (B) DMEM/F12 | | 93 | NA | 93 | NA |
| | with 1% HSA | | | | | |
| | (C) NUT (−) | | 95 | NA | 95 | NA |
| | with HSA | | | | | |
| TAI#13 | | 35 | 96 | NA | 96 | NA |
| DEVOPCTest#25 | F_CoolCell | 42 | 92 | 92 | 105 | 95 |
| | G_CryoMed | | 87 | 90 | 104 | 105 |

NOTE:
Due to the large cell quantity required to generate the desired TAI DP, depending on vessel size, obtaining two TAI vials was not always attainable.

% Viability range of thawed DP TAI cells obtained from DS neutralized with GPM (+) was 88%-98% in all tests, and % recovery range in these tests was 90%-117% (table 6). This relatively wide range of % recovery is attributed to the accumulated errors when dealing with high cell concentrations combined with very small working volumes of DP—usually a few hundred μl—that were used in each of these tests to generate the final DP formulation of the desired concentration. TAI #7 showed that cells harvested on Day35 from LN521-coated flasks generate TAI vials that are as good as Day42 TAI vials in respect to % viability and % recovery. However, a marginally lower PDGFRα marker expression value (table 6) required these results be corroborated in TAI #13, with cells harvested from LN521-coated 1-layer CF. Indeed, TAI #13 results met all acceptance criteria. Having demonstrated the ability to generate TAI DP at day 35 from LN521-coated flasks-derived cells, TAI #11, TAI#12 and DEVOPC-Test #25 demonstrated the feasibility of generating TAI DP when using iMatrix511-E8-supplemented media, with % recovery and % viability results as good as LN521-coated flasks-derived DP cells. TAI #6 and TAI #12 aimed to evaluate the feasibility of replacing the enzyme neutralization solution with either DMEM/F12 (as is in TAI #6 or supplemented with 1% HSA in TAI #12) or NUT (−) with HSA.

Results show that both GPM (+) and NUT (−) with HSA gave highly similar % viability and % recovery results. Marker expression analyses also showed that NUT (−) with HSA was comparable to the currently established GPM (+) enzyme neutralization solution, with all relevant markers meeting current acceptance criteria (table 6). Furthermore, it is apparent that using DMEM/F12—as is or supplemented with 1% HSA, group B in TAI #6 and TAI #12—as the enzyme neutralization solution decreases post-thawing % viability of cells by approx. 8% yet does not largely affect % recovery. DEVOPC-Test #25 in which NUT (−) with HSA was also applied as the TS neutralizing solution further corroborated this data.

Tables 5 and 6 below show marker expression and Decorin Secretion analyses results.

TABLE 5
Marker expression analyses results

| Test #/(group) | | Harvest Day | PDGFRα | NG2 | PDGFRβ | A2B5 | GD3 | p75 |
|---|---|---|---|---|---|---|---|---|
| TAI#3 | | 42 | 98.7 | 93.1 | 99.1 | 4.81 | 97.8 | NA |
| TAI#4 | | 42 | 98.8 | 95.5 | 99.4 | 4.19 | 95.5 | NA |
| TAI#5 | | 42 | 98.2 | 89.9 | 98.8 | 15.6 | 93.1 | 95.6 |
| TAI#6 | (A) GPM+ | 42 | 97.8 | 93.9 | 97.9 | 13.6 | 90.7 | 91.6 |
| | (B) DMEM/F12 | | 98.7 | 90.3 | 98.2 | 16.2 | 81.5 | 91.7 |
| | (C) NUT (−) | | 98.4 | 93.4 | 98.3 | 15.1 | 86.4 | 90.8 |
| | with HSA | | | | | | | |
| TAI#7 | | 35 | 93.3 | 91.5 | 95.2 | 57.7 | 97.7 | 87.3 |
| TAI#8 | | 42 | 98.4 | 96.6 | 98.8 | 19.0 | 93.8 | 96.4 |
| TAI#11 | | 42 | 97.7 | 81.0 | 98.2 | 24.6 | 87.4 | 95.5 |
| TAI#12 | (A) GPM+ | 42 | 98.9 | 96.9 | 97.4 | 15.3 | 98.4 | 93.3 |
| | (B) DMEM/F12 | | 97.8 | 89.9 | 97.1 | 17.6 | 98.3 | 91.4 |
| | with 1% HSA | | | | | | | |
| | (C) NUT (−) | | 97.3 | 92.0 | 96.9 | 18.7 | 98.8 | 92.3 |
| | with HSA | | | | | | | |
| TAI#13 | | 35 | 98.4 | 95.0 | 98.2 | 17.0 | 96.6 | 92.0 |
| DEVOPCTest#25 ⁴ | F_Cool Cell | 42 | 98.6 | 93.2 | 97.9 | 6.1 | 86.1 | 92.8 |
| | G_CryoMed | | 97.9 | 95.9 | 98.3 | 7.78 | 89.1 | 92.8 |

| | | | | | | | | Beta |
|---|---|---|---|---|---|---|---|---|
| | | | | | TRA- | | | Tubulin |
| Test #/(group) | | Claudin6 | EpCAM | CD49f | 1-60 | GFAP | CK7 | III |
| TAI#3 | | 0.00 | 0.56 | NA | 0.07 | NA | 0.36 | NA |
| TAI#4 | | 0.10 | 0.59 | NA | 0.031 | NA | 0.47 | NA |
| TAI#5 | | 0.084 | 0.31 | 1.57 | 0.15 | 0 | 0.58 | 1.31 |
| TAI#6 | (A) GPM+ | 0.094 | 0.81 | 1.31 | 0.22 | 0.22 | 0.94 | 1.85 |
| | (B) DMEM/F12 | 0.075 | 0.55 | 0.94 | 0.12 | 0.15 | 0.92 | 1.66 |
| | (C) NUT (−) | 0.11 | 0.65 | 0.97 | 0.13 | 0.065 | 0.78 | 1.42 |
| | with HSA | | | | | | | |
| TAI#7 | | 0.44 | 0.93 | 6.09 | 0.17 | 0.019 | 0.037 | 0.73 |
| TAI#8 | | 0.22 | 0.72 | 2.12 | 0.22 | 0.14 | 0.27 | 0.11 |
| TAI#11 | | 0.45 | 1.02 | 2.28 | 0.43 | 0.32 | 0.018 | 0.78 |
| TAI#12 | (A) GPM+ | 0.38 | 1.76 | 11.7 | 0.33 | 0.46 ¹ | 0.42 ¹ | 5.63 ¹ |
| | (B) DMEM/F12 | 0.30 | 1.47 | 4.23 | 0.25 | 0.24 ¹ | 0.40 ¹ | 4.42 ¹ |
| | with 1% HSA | | | | | | | |
| | (C) NUT (−) | 0.33 | 1.45 | 5.25 | 0.30 | 0.22 ² | 0.25 ² | 3.49 ² |
| | with HSA | | | | | | | |
| TAI#13 | | 0.38 | 0.96 | 1.6 | 0.10 | 0.042 ³ | 0.47 ² | 3.37 ³ |
| DEVOPC | F_Cool Cell | 0.044 | 0.21 | 0.6 | 0.11 | 0.025 | 0.43 | 1.12 |
| Test#25 ⁴ | G_CryoMed | 0.039 | 0.29 | 0.76 | 0.082 | 0.026 | 0.49 | 1.89 |

NOTE:
GFAP, CK7 and Beta Tubulin III marker expression values in tests TAI#12 and TAI#13 were derived from a relatively small number of FACS events as detailed hereafter:
¹ low live cell number (>2000, <5000);
² low live cell number (>1000, <2000);
³ low live cell number (>400, <1000)
⁴ Group G: cells filtered using a 60 μm filter kit and cryopreserved either in the CryoMed or a CoolCell.

Marker expression values, including impurity markers, met their respective acceptance criteria in all tests, except PDGFRα expression in TAI #7 which was slightly below 95.00% (Table 5). These results correlate with the original RD run; therefore, this slight decrease is not attributed to the OPC1 TAI cryopreservation method.

Although NG2 expression seemed to vary among tests, it has been determined that its expression is highly affected by the harvesting procedure hence a reduction in NG2-positive cells is not necessarily indicative of potential loss of OPC identity, especially having established that NG2 expression is fully recovered following an overnight culturing session; GMP batches are be assayed for NG2 expression following an overnight culturing session.

TABLE 6
Decorin secretion assay results

| | | | Decorin |
|---|---|---|---|
| Test # | | Harvesting | Secretion |
| (group) | | Day | (ng/ml) |
| TAI#5 | | 42 | 33.40 |
| TAI#6 | (A) GPM+ | 42 | 26.42 |
| | (B) DMEM/F 12 | | 23.54 |
| | (C) NUT (−) with HSA | | 27.88 |
| TAI#8 | | 42 | 29.03 |
| TAI#11 | | 42 | 42.68 |
| TAI#12 | (A) GPM+ | 42 | 40.79 |
| | (B) DMEM/F 12 with 1% HSA | | 39.95 |
| | (C) NUT (−) with HSA | | 36.41 |
| TAI#13 | | 35 | 33.20 |
| DEVOPC | F_CoolCell | 42 | 29.50 |
| Test#25 | G_CryoMed | | 27.59 |

TAI#3 and TAI#4 were not assayed for Decorin secretion.

Decorin secretion in all tests and conditions has met acceptance criteria (>20.00 ng/ml., table 8). Note that Decorin secretion values of TAI #6 were relatively lower in all three conditions compared with the other tests, which leads to assume that this can be attributed to cell source rather than a direct result of test conditions, as further evident from TAI #12 which applied the identical test conditions as TAI #6.

### Discussion

Establishing the final formulation of OPC TAI DP generated in the optimized OPC differentiation process Stage II, concurrent with a method for thawing TAI vials, has been the focus of this study.

Having the OPC DP engaged in a clinical study required formulating the OPC1 TAI DP while maintaining its target live cell concentration of 100×10⁶ cells per ml., which allows for a clinical dose of 10×10⁶ cells in 100 μl. Hence, each clinical vial contains 30×10⁶ live cells in 300 μl of CS10 cell suspension.

A robust and simple method for thawing OPC1 TAI vials to conduct the clinical study was a prerequisite for allowing further analyses of OPC1 TAI cells during the course of this study. Hence, the standard thawing method using a 37° C. water bath was compared to a 37° C. dry bath using an MD-Mini device.

Simulating clinical TAI vials with 300 μl CS10-filled 0.5 ml NUNC vials, thawing using the MD-Mini dry bath was established as a 2-minute procedure which was shown to be equivalent to a 1 min. 25 sec. thawing procedure in the 37° C. water bath (with vials having a visible sliver of ice at the end of the thawing time). Subsequently, all TAI vials in this study were thawed in the MD-mini dry bath. Additionally, the incubation time of thawed cells in the required dilution for NC-200 analyses was established as 2 minutes at RT in GPM (+).

Initial qualification of the OPC TAI DP was established with cells differentiated and cultured using LN521-coated vessels with mTeSR1 in OPC differentiation process Stage I and GPM (+) as culture media as per OPC differentiation process Stage II (tests TAI #3 and TAI #4, table 3). These proved the feasibility to generate TAI cells that meet all acceptance criteria, including marker expression and potency (Decorin secretion), in the newly optimized OPC differentiation process Stage II, thus paving the way for further process scale-up.

Demonstrating the feasibility of using a cell-dissociating enzyme neutralization solution which will not only maintain OPC1 TAI DP quality attributes but also allow for the elimination of non-clinical grade and animal derived media components (B27 and T3 supplements) on harvesting day was the aim of TAI #6 and TAI #12 (Table 3). Comparing 3 solution types, it has been shown that using NUT (−) with HSA was as good as the standard GPM (+) for neutralizing the cell dissociating enzyme, with cell quality attributes meeting all acceptance criteria (tables 4 and 5) and % viability and % recovery results as good as those obtained when using GPM (+) for that purpose (table 4).

Evaluating the feasibility of manufacturing OPC1 TAI DP generated from OPC cells that originated from a Day14 ICB culture and harvested on Day42 was the aim of TAI #11. Results showed that cell quality attributes were on par with those of cells obtained from an ongoing culture harvested on Day42 (tables 4 and 5).

Tests were also executed for evaluating TAI DP formulated using cells harvested on Day 35 and comparing their quality attributes with those of OPC1 TAI DP formulated with cells harvested on Day 42. Results of TAI #7 and TAI #13 have shown that these cells met all acceptance criteria (albeit TAI #7 exhibiting a slightly lower PDGFRα marker expression compared to its source culture). Furthermore, day35 TAI DP generated in TAI #13 not only corroborated the quality of cells obtained from Cell Factory vessels on Day 35 but also the benefits of using NUT (−) HSA as the cell-dissociating enzyme neutralization solution during harvesting.

Finally, test DEVOPC_Test #25 demonstrated the robustness and repeatability of the scaled-up OPC differentiation process Stage II as a wholly complete process as this test encompassed the cell expansion process in CF applying the direct-coating method using iMatrix511-E8 together with both the optimized scaled-up filtration procedure using a 60 μm filter kit and the OPC1 TAI DP cryopreservation process using the CryoMed. Both marker expression and Decorin secretion values of OPC1 TAI generated from 60 μm-filtered cells that were cryopreserved in the CryoMed met acceptance criteria.

All in all, the data presented in this study support and establish the OPC1 TAI DP as a cell suspension of 100×10⁶ live cells per ml CS10, in a final volume of 300 μl per 0.5m1NUNC vial. Moreover, this study corroborate the ability of the scaled-up manufacturing process of OPC to generate OPC1 TAI DP from cells cultured in direct-coated 1-layer CF using iMatrix511-E8-supplemented media, harvested—either on Day35 or Day42—with TS and NUT(−) with HSA as the TS neutralizing agent, and filtered via the 60 μm filter kit before cryopreservation in the CryoMed. Cells obtained in this optimized scaled-up manufacturing process have met OPC1 acceptance criteria and demonstrated the functional biological activity of OPC1 TAI.

OPC TAI is established as a 100×10⁶ live cells/ml. suspension in CS10; TAI vials containing 30×10⁶ live cells per 300 μl, in 0.5 ml vials. Thawing OPC TAI vials for 2 minutes in a 37° C. MD-Mini dry bath is as good and as robust as thawing in a 37° C. water bath. OPC1 TAI DP generated from cells cultured in direct-coated 1-layer CF using iMatrix511-E8-supplemented media is as good as OPC1 TAI DP obtained from cells cultured in LN521-pre coated vessels. Harvesting cells on Day35 of the OPC differentiation process Stage II for generating TAI DP is feasible under the aforementioned process conditions. NUT (−) with HSA as cell-dissociating enzyme neutralization solution is as good as GPM (+) for generating OPC1 TAI DP from cells harvested either on Day 35 or Day42. Day14 ICB has been shown to generate OPC1 TAI DP displaying quality attributes that are on par with the DP obtained from an ongoing cell culture. OPC1 TAI DP manufactured in the optimized scaled-up manufacturing process using the 60pm filter kit and cryopreserved in the CryoMed meets all acceptance criteria and exhibits functional biological activity.

### Example 2: OPC1 for One-Time Administration for the Treatment of Subacute Spinal Cord Injury (SCI)

The purpose of this Example is to present the scientific data generated during the development of OPC1 (also referred to as LTCOPC1) CMC program. The provided information includes the development plans for LCTOPC1 with regards to preliminary comparability results based on R&D runs of the improved manufacturing process, comparability between the GPOR and LCT R&D manufactured material, introduction of a new proposed potency assay, review of the OPC1 safety status based on the GPOR in vivo data and reanalysis of GPOR lots, utilizing improved analytical methods.

LCTOPC1 (OPC1), previously referred to as GRNOPC1 and AST-OPC1, is an oligodendrocyte progenitor cell population derived from the H1 hESC line intended for one-time administration for the treatment of subacute spinal cord injury (SCI). OPC1 has been shown in pre-clinical studies to produce neurotrophic factors, migrate in the spinal cord parenchyma, stimulate vascularization, and induce remyelination of denuded axons, all of which are essential functions of oligodendrocyte progenitors and are important for survival, regrowth and function of axons.

Clinical evaluation of LCTOPC1 was initiated in 2010. The first clinical trial was a Phase 1 safety study (NCT01217008) in which a low dose of 2×10⁶ OPC1 cells was injected into the lesion site of subjects with subacute, neurologically complete thoracic spinal T3-T11 injuries. A total of 5 subjects out of the planned 8 received OPC1 as part of the original Phase 1 CP35A007 safety study from October 2010 through November 2011. Enrollment in this trial was halted in November 2011.

In 2014, a Phase 1/2a study was initiated (NCT02302157) dose escalation of OPC1 in subjects with subacute sensorimotor complete (American Spinal Injury Association Impairment Scale A (ASIA Impairment Scale A)), Single Neurological Level (SNL) from C5 to C7 cervical spinal cord injuries, with the later expansion of the study to patients with a C4 Neurological Level of Injury (NLI) if the Upper Extremity Motor Score (UEMS)≥1 and changing the dosing window from 14-30 days to 21-42 days post-spinal cord injury. A total of 25 subjects across 5 cohorts were enrolled in the study and received a single administration of OPC1 cells delivered by intra-parenchymal injection into the spinal cord injury site using a Syringe Positioning Device, during a dedicated surgical procedure. The enrollment for the study was completed in December 2017.

In November 2018 a randomized, controlled, single-blind Phase 2 study was proposed to FDA and the Agency had no objection to use OPC1 Drug Product lot M26D1A in the proposed Phase 2 clinical study.

Briefly, the origin of the new Master Cell Bank (MCB) is the H1 Bank Lot. No. MCBH101. MCBH101 was manufactured by Geron Corporation (Geron; Foster City, CA) directly from the H1 Original Cell Bank (OCB) in 2009. The new MCB originated directly from the H1 Bank-Lot. No. MCBH101. It was manufactured in feeder-free conditions using well-defined raw materials, new culturing system and harvesting procedure, and cryopreserved by an hESC-customized cryopreservation process. In addition, the method for assessment of H1 hESC pluripotency was optimized. The new WCB originated from the new MCB and was expanded in tissue culture for 4 passages, while maintaining hESC characteristics, and then cryopreserved. Starting material for LCTOPC1 manufacturing is provided by the WCB. The new Master and Working Cell Banks were released according to predefined release criteria and tested for adventitious agents using an approved testing laboratory according to approved study protocols, generally prevailing industry standards (ICH Q5A (R1), Q5D), and Current Manufacturing Practices (“Regulations”) and laws applicable to the Study being performed, as amended from time to time.

OPC1 is an investigational drug studied in a Phase 1 and a Phase 1/2a spinal injury clinical studies using OPC1 clinical lots produced by Geron, manufactured between 2008 and 2011. Geron's manufacturing process (GPOR) was originally developed in the early 2000 s. At that time, well-defined and cell therapy grade reagents and materials were not widely available. As such, Stage 1 of the GPOR manufacturing process included the propagation of HI embryonic cells on Matrigel™, an animal derived Extracellular Matrix (ECM), collagenase, and manual scraping of the culture dish surface for harvesting, passaging and expansion of the HI embryonic stem cells.

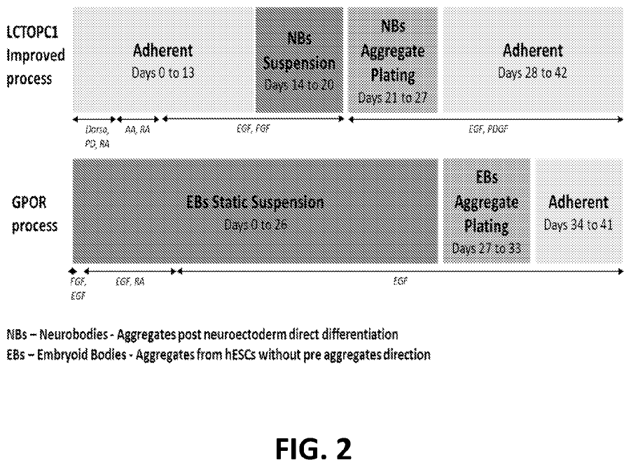

Furthermore, the GPOR manufacturing process was based on a differentiation process which occurred in cell aggregates starting directly from pluripotent H1 cells, in the form of Embryoid bodies (day 0 to day 26, FIG. 2), which have a strong susceptibility to spontaneous differentiation. From day 27 on, the differentiation was completed on Matrigel™ coated adherent surface for oligodendrocyte progenitor expansion and maturation. The GPOR manufacturing process thus had low yields, and key quality attributes defined by purity/impurity/non-targeted cell population markers exhibited limited reproducibility. Additionally, the final cryopreserved product required upon thawing, washing and formulation preparation prior to administration.

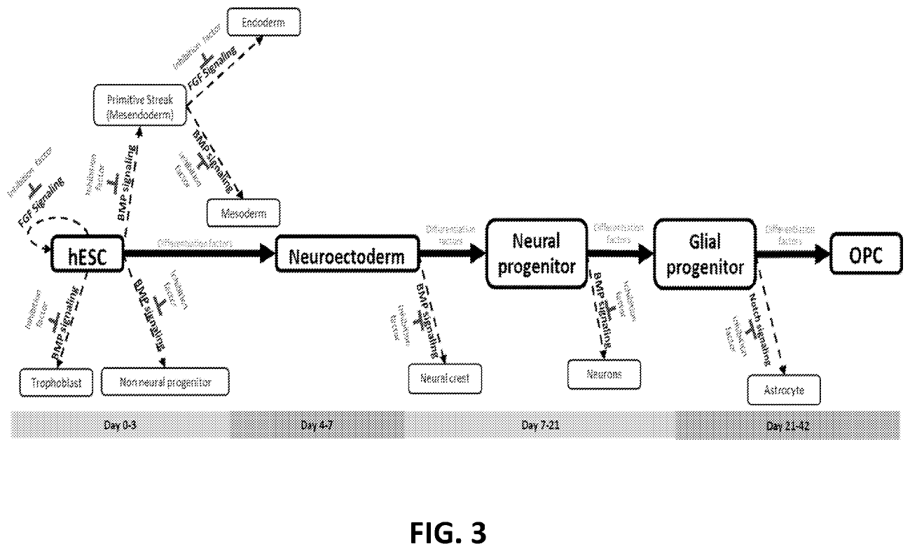

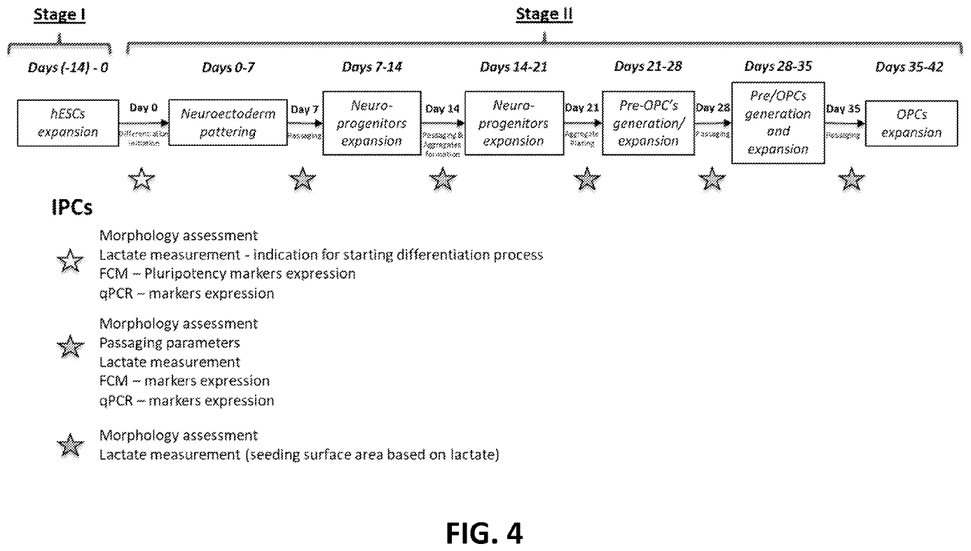

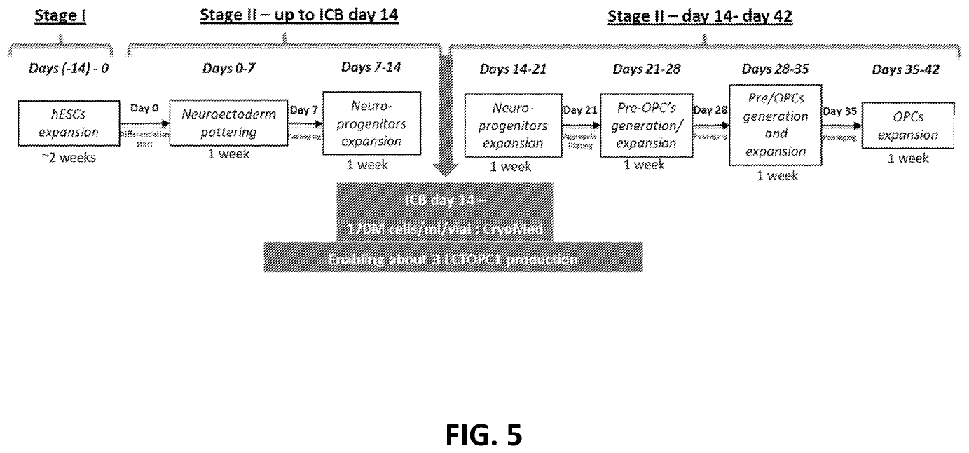

The development of the improved manufacturing process in accordance with the present disclosure is a more controlled directed differentiation of H1 towards OPC, guided by a specific sequence of growth factors and small molecules to inhibit or direct differentiation pathways using cell therapy grade materials when possible (as detailed in FIG. 3). Moreover, the new process reduces the lengthy aggregate phase used by Geron, from 26 days directly from pluripotent cell state, which is prone to spontaneous aberrant differentiation, to 7 days, following 14 days of monolayer directed differentiation of H1 cells into neuroectoderm, reducing the possibility for spontaneous differentiation in the aggregates phase. The GPOR vs. LCTOPC1 differentiation processes are summarized in FIG. 2. The biological rationale for the signaling sequence of inducing and inhibitory factors of the improved differentiation process is described in FIG. 4. Additionally, new in-process controls (IPCs) were added to better monitor and characterize the differentiation process, as detailed in FIG. 4.

Finally, the product is cryopreserved as a ready-to-inject, referred to herein as “Thaw and Inject (TAI)”, OPC1 formulation. Materials used to manufacture OPC1 cells (both the original GPOR and the modified processes) are summarized in Table 7 below.

TABLE 7
Materials used during the production of
OPC cells (GPOR and LCTOPC1 processes.

| | Parameter | GPOR Process | LCTOPC1 Process |
|---|---|---|---|
| Stage I - | Culturing | XVIVO 10 | mTeSR Medium |
| hESCs H1 | System | Medium | ReleSR |
| expansion | | Collagenase/ | rhLaminin |
| | | mechanical | |
| | | scraping | |
| | | Matrigel | |
| Stage II - | Differentiation | GPM (DMEM/F12, T3, B-27) | |
| hESCs H1 | Medium | | |
| differentiation | Coating Reagent | Matrigel | rhLaminin |
| into OPC | Growth and | rhEGF | Dorsomorphin |
| | Differentiation | rhbFGF | PD0325901 |
| | Factors | Retinoic | Retinoic Acid (RA) |
| | | Acid (RA) | Ascorbic Acid (AA) |
| | | | rhEGF |
| | | | heat stable rhbFGF |
| | | | ROCK Inhibitor |
| | | | PDGF-A |

### Stage I—H1 Expansion

Pluripotent H1 cells are thawed and cultured for 12-15 days on laminin-coated vessels in mTeSR Plus Medium. The cells are passaged and expanded using a non-enzymatic reagent ReLeSR. During the expansion, the cells are morphologically assessed, and at the end of 3 passages (before the initiation of differentiation process), hESC pluripotency is evaluated by flow cytometry-based method.

### Stage II—H1 Differentiation into OPC1

From day 0 of differentiation until the end of the process, the cells are cultured in Glial Progenitor Medium (GPM)—which is DMEM/F-12 supplemented with B27 and T3.

Day 0-7—on day 0, when the H1 culture reaches the required criteria which is defined by lactate concentration and cell morphology, the differentiation process is initiated by changing the medium for the expanded pluripotent hESC cultured on laminin-coated vessels as follows. On days 0-3, GPM medium is supplemented with Retinoic Acid (RA), Dorsomorphin and PD0325901, in order to direct the differentiation process towards the neuroectoderm pathway (Kudoh, Wilson et al. 2002). Dorsomorphin inhibits Bone Morphogenic Protein (BMP) signaling (SMAD pathway) and therefore inhibits mesoderm and trophoblast differentiation (Li and Parast 2014). PD0325901 inhibits downstream bFGF signaling at MEK1 and MEK2, and inhibits pluripotency and endoderm differentiation (Sui, Bouwens et al. 2013). In summary, inhibition of pluripotency, endoderm, mesoderm and trophoblast formation in addition to activation of the RA signaling pathway, promotes neural tube (neuroectoderm) formation (Watabe and Miyazono 2009, Sui, Bouwens et al. 2013, Li and Parast 2014, Patthey and Gunhaga 2014, Janesick, Wu et al. 2015). On days 4-7, the culture is supplemented with Retinoic Acid and Ascorbic Acid to continue neuroectoderm differentiation induction (Duester 2008).

Day 7-14—On day 7 the cells are enzymatically harvested using TrypLE Select, and then seeded as a monolayer culture from day 7 to day 14 on laminin-coated vessels and cultured in GPM supplemented with rhEGF, hsFGF and ROCK inhibitor (ROCK Inhibitor only for the first 48 hours) (Hu, Du et al. 2009, Patthey and Gunhaga 2014, Zheng, Li et al. 2018).

Day 14-21—On Day 14, in order to promote neurobody (NB) aggregate formation, the cells are enzymatically harvested using TrypLE Select, and cultured for a week as a dynamic suspension culture in GPM supplemented with ROCK inhibitor (for the first 48 hours), and rhEGF and hs-rhFGF throughout.

Day 21-42—On Day 21 the aggregates are plated back as an adherent culture on laminin-coated vessels in GPM supplemented with rhEGF and PDGF (Ota and Ito 2006, Koch, Lehal et al. 2013), and then on Day 28, the cells are harvested enzymatically using TrypLE Select, and expanded as an adherent culture on laminin-coated vessels in GPM supplemented with rhEGF and PDGF until days 35-42 for final expansion and maturation, with enzymatic passaging every ˜7 days using TrypLE Select.

At the end of the expansion process, the OPC1 cells are harvested using TrypLE Select and cryopreserved in CryoStor® 10 (CS10; BioLife Solutions, Inc.) cryopreservation solution as a Thaw-and-Inject (TAI) formulation. The LCTOPC1 production process flow is depicted in FIG. 4.

In-Process Control tests are performed at every key step during the differentiation process of hESC to OPC1, as depicted in FIG. 4. Biomarker proteins and mRNA expression are assessed using multicolor Flow Cytometry (FCM) and qPCR methods (respectively). The cells are tested for the expression of OPC1, epithelial, mesodermal, astrocytes and neuronal biomarkers, and residual hESC. In addition, viability, cell yield and metabolic activity (e.g., lactate) are assessed during the process. Lactate concentration is used as indicator for initiating differentiation starting on day 0, and on day 21 as a surrogate to cell counting in order to determine the surface area required for aggregate plating for pre-OPC generation and expansion.

### Cryopreservation of LCTOPC1 DP as Thaw-and-Inject Formulation

The improved LCTOPC1 Drug Product (DP) is being developed as a Thaw-and-Inject (TAI) formulation, to be thawed and immediately loaded into the delivery device before injection in the spinal cord. This improvement significantly reduces risks associated with additional manipulations at the clinical site such as washing and resuspension for final formulation prior to administration, and increases the control over product consistency, safety and quality.

The development of TAI DP formulation includes using CryoStor® 10 (CS10) as the cryopreservation media for generating clinical dosages targeted to 100×10⁶ viable cells per mL with a final volume of 300 μL cell suspension per vial and focuses on: evaluation of the optimal cell dissociating enzyme neutralization solution and the addition of a filtration step prior to cryopreservation; establishment of OPC1 TAI formulation targeted to 100×10⁶ viable cells per mL in CS10, with a final volume of 300 μL, cell suspension per vial; establishment of the thawing procedure for OPC1 TAI vials containing 300μL; and confirming that the optimized scaled-up OPC1 improved manufacturing process generates OPC1 TAI which meets all acceptance criteria.

Development studies support and establish the LCT OPC1 TAI DP as a cell suspension targeted to 100×10⁶ viable cells per mL CS10, in a final volume of 300 μL in 0.5 mL NUNC vials. Moreover, the studies demonstrate the ability of the scaled-up manufacturing process of LCT OPC1 to generate OPC1 TAI DP, harvested with TrypLE Select and NUT(−) with HSA as the TrypLE Select neutralizing agent, and filtered via a 60 μm filter kit before cryopreservation in the CryoMed controlled-rate freezer. Cells obtained in this optimized and scaled-up manufacturing process show acceptable quality attributes, viability and recovery, and functional biological activity.

### Control of Materials

Control of ancillary materials. Each ancillary material used for manufacturing of LCTOPC1 is considered based on process development studies and individual qualification requirements, based on a Risk Level assessment (USP NF<1043>) and the Criticality of the material for product and/or process quality. Risk Level (RL) is determined by a risk assessment procedure to evaluate manufacturer's quality system, safety risk (origin of the material and manufacturing process-related risk), and material grade (quality standard). Criticality to the process/product is determined by assessment of the material impact to the production procedure and/or product quality.

Non-cellular Impurities. Calculations of estimated amounts of potential non-cellular impurities in LCTOPC1 DP is based on procedures done before the manufacturing of the final DP formulation and the DP dose. Risk assessment is conducted based on the theoretical calculation of non-cellular impurity levels, material biosafety, and pharmacological risks. Based on the risk assessment outcomes, further mitigation is implemented such as analytical testing of DP for detection and measurement of specific non-cellular impurities, material removal/reduction from DP, and extended biosafety tests as required.

### Proposed CMC Comparability Testing

OPC1 is manufactured at a GMP facility according to the improved process, released according to revised release parameters, and cryopreserved in the developed TAI formulation. LCTOPC1 DP is compared to Geron's manufactured representative batches and characterized with the updated analytical methods used for the release of the OPC1 manufactured via the new process. The plan includes testing of attributes used as release criteria for GPOR plus additional markers that were identified. The Sponsor suggestion for comparison is based on quality attributes that characterize the Drug Product as described in Table 8.

The side-by-side comparison between LCTOPC1 and GPOR OPC1 batches are based on statistical analysis, calculating the expected range for quantitative measurements of the quality attributes from GPOR OPC1 batches. The values of those quality attributes measured in LCTOPC1 batches are assessed in relation to those expected ranges for the quality attributes tested. The comparability data analysis is expected to establish reproducible release criteria for the LCTOPC1 process and demonstrate that LCTOPC1 has low batch to batch variability.

The tested quality attributes include viability, identity/purity, impurity population, and function/potency assays for 3-4 representative GPOR and LCT OPC1 batches each.

Suggested comparability quality attributes are as follow: Viability—a critical quality attribute of any live cell drug product; Identity/Purity—assessment of characteristic oligodendrocyte progenitor cell protein markers: NG2, PDGFRα and PDGRFβ; Non-targeted cells population/impurities; and In-vitro Cells Function/Potency.

Regarding non-targeted cells impurity population. Residual H1 hESC from starting material—human embryonic stem cells protein markers TRA-1-60 and Oct-4 as a potential source for teratogenic agents combined in a multi-color Flow Cytometry test. TRA-1-60 and Oct-4 are commonly known and used markers for embryonic stem cells and were used previously as an OPC1 release criteria. Assessment of potential aberrant differentiation paths. Epithelial cells protein markers: Keratin 7, Claudin-6, EpCAM and CD49f known epithelial markers. Mesodermal cells protein markers CXCR4 and CD56 as known mesodermal and cartilage markers. Astrocytes cells protein marker GFAP as known astrocytes marker. Mesenchymal cells mRNA OLR1 that induces epithelial-mesenchymal transition. Endoderm cells mRNA markers of FOXA2, SOX17 as known endodermal markers. At present it is not planned to include previously used Nestin and α-actinin attributes, since new data indicate that Nestin is not specific for OPC, but rather a marker for NSC and other cell types (see website ncbi.nlm.nih.gov/pmc/articles/PMC5948302/) and α-actinin can be effectively replaced by combination of CXCR4/CD56. CXCR4 is expressed in definitive endoderm and mesoderm: see website pubmed.ncbi.nlm.nih.gov/16258519/ and website pnas.org/content/107/31/13742.

Regarding in-vitro cells function/potency. Decorin secretion—a small secreted cellular matrix proteoglycan, as a potency indicator for OPC1. Decorin expressed by neurons and astrocytes in the central nervous system attenuates scar tissue formation, inhibits cavitation and promotes wound healing. OPC1 cells migration in response to platelet-derived growth factor-BB (PDGF-BB) which is important for cell motility at the injury site to ensure broadest anatomical coverage from a single injection location in the spine. Development of new potency test for assessment of the maturation and myelinization potential of OPC1. This assay is based on an essential function of OPC1 cells-remyelination of denuded axons. In this assay, OPC1 cells are thawed and plated in a specific media in a 3D tissue culture environment (e.g., Matrigel or Nanofiber tubes) that should induce the maturation of OPC1 into Oligodendrocytes (OL). The 3D environment nanotube mimics denuded axons in order to induce myelinization activity by OPC1 cells in a simple in-vitro setting. The assay measures expression of proteins associated with OL function and maturation state such as MBP, O4, MAG, MOG, PLP, CNpaseby immunocytochemistry, flow cytometry and qPCR.

The proposed test panel to be used to examine GPOR OPC1 and LCTOPC1 along with proposed release criteria for LCTOPC1 can be seen below in Table 8.

TABLE 8
The proposed test panel and rationale used for process
development, release, and comparability.

| | | Comparability |
|---|---|---|
| | | quality |
| Test | Specification | attribute |
| Cell Viability | ≥70% | Yes |
| Strength | 0.75-1.25 × 10⁸ | No |
| | Live cells/ml | |

Biosafety

| | | |
|---|---|---|
| Sterility | No growth | No |
| Mycoplasma | No growth | No |
| Endotoxin | <2 EU/ml | No |
| Karyotype | Less than 3 repeat | No |
| | deletions or 2 | |
| | repeat additions to | |
| | the chromosomes | |

Purity/Identity

| | | |
|---|---|---|
| % NG2+ | ≥60% | Yes |
| % PDGFRa+ | ≥90% | Yes |
| % PDGFRβ+ | ≥90% | Yes |

Impurities

| | | | |
|---|---|---|---|
| Flow | % Keratin 7+ | ≤2% | Yes |
| Cytometry | % of Claudin-6+ | ≤2% | Yes |
| | % of EpCAM+ | ≤2% | Yes |
| | % CD49f | ≤5% | Yes |
| | % TRA-1-60+ | ≤1% | Yes |
| | % TRA-1-60+/ | ≤0.01% | Yes |
| | Oct-4+ | | |
| | % GFAP | ≤2% | Yes |
| | % CXCR4+/CD56+ | ≤5% | Yes |
| qPCR | OLR1 | Report | Yes |
| | FOXA2 | Report | Yes |
| | SOX17 | Report | Yes |

Function/Potency

| | | |
|---|---|---|
| Decorin Secretion | ≥ 15 ng/ml | Yes |
| Cell Migration | ≥30% | Yes |
| | (+PDGFββ) | |

Preliminary comparability of R&D LCTOPC1 batches, from representative runs are presented below in Tables 9-13.

TABLE 9
Comparability data from representative GPOR OPC1 and LCTOPC1
R&D Runs -OPC1 Identity profile by flow cytometry

| Current | Proposed | | GPOR OPC1 | | | LCTOPC1 | | |
|---|---|---|---|---|---|---|---|---|
| Marker | Marker | Justification | M08¹ | M22¹ | M25¹ | RD11A2 | RD11C | RD12D1 |
| % NG2 | % GD3 | Known Pre-OPC marker | 85.4 | 89.9 | 86.4 | 90 | 87.1 | 88 |
| % Nestin | % NG2 | Known OPC marker | 47.8 | 27.8 | 18.2 | 88 | 90.5 | 95.3 |
| | % NG2 (*MG) | | 91.6 | 90.7 | 91.2 | 93.9 | 91.3 | 92.7 |
| | % PDGFRα | Known OPC marker | 42.3 | 52.2 | 55.8 | 98.9 | 98.4 | 99.2 |
| | % PDGFRα | | 79.7 | 79.9 | 87 | 99 | 99 | 99.3 |
| | (*MG) | | | | | | | |
| | % NG2/PDGFR | Known OPC markers | 27 | 16.2 | 11.8 | 86.5 | 89.1 | 94.9 |
| | % NG2/PDGFRα | | 78.5 | 78 | 83.8 | 93.4 | 91.1 | 92.5 |
| | (*MG) | | | | | | | |
| | % PDGFRβ | Essential for | 92.7 | 87.1 | 91.6 | 99.1 | 98.7 | 99.1 |
| | | maturation of | | | | | | |
| | | oligodendrocytes | | | | | | |

*MG—Cells are seeded on Matrigel overnight before FCM analysis. GPOR OPC1 formulation and freezing reduces NG-2 and PDGFR-a expression. Matrigel overnight culture as previously shown and now by these data.
¹Clinical batch

TABLE 10
Comparability data from representative GPOR and LCTOPC1 R&D Runs -
non-targeted/impurities cell population profile by flow cytometry

| Cell | Current | Proposed | | GPOR OPC1 | | | LCTOPC1 | | |
|---|---|---|---|---|---|---|---|---|---|
| population | Marker | Marker | Justification | M08¹ | M22¹ | M25¹ | RD11A2 | RD11C | RD12D1 |
| Epithelial | % Keratin 7 | % Keratin 7 | Known epithelial | 1.04 | 1.47 | 1.88 | 0.25 | 0.38 | 0.36 |
| | | % Claudin-6 | markers | 4.45 | 5.83 | 3.09 | 0.33 | 0.42 | 0.17 |
| | | % EpCAM | | 1.17 | 2.21 | 1.37 | 0.62 | 0.72 | 0.99 |
| | | % CD49f | | 24.3 | 21.2 | 23.6 | 0.83 | 1.26 | 2.66 |
| Astrocytes | % GFAP | % GFAP | Known astrocytes | 1.78 | 3.44 | 1.62 | 0.028 | 0.04 | 1.35 |
| | | | marker | | | | | | |
| Neuronal | % b- | % β- | Known | 13.7 | 17.9 | 14.3 | 0.51 | 0.29 | 0.87 |
| Phenotype | Tubulin III | Tubulin III | Neuronal marker | | | | | | |

¹Clinical batch

TABLE 11
Comparability data from representative GPOR OPC1 and
LCTOPC1 R&D Runs - hESC residuals by flow cytometry

| | | | | | | | LCTOPC1 | | |
|---|---|---|---|---|---|---|---|---|---|
| Cell | Current | Proposed | | GPOR OPC1 | | | RD | RD | RD |
| population | marker | marker | Justification | M08¹ | M22¹ | M25¹ | 11A2 | 11C | 12D1 |
| hESCs | % TRA-1- | % TRA-1- | Teratogenic | 0.01 | 0.00 | 0.00 | 0.00 | 0.00 | 0.00 |
| | 60+/Oct4+ | 60+/Oct4+ | potential | | | | | | |
| hESCs | % TRA-1- | % TRA-1- | Teratogenic | 0.41 | 0.49 | 0.44 | 0.01 | 0.04 | 0.01 |
| | 60+/Oct4− | 60+/Oct4− | potential | | | | | | |

¹Clinical batch

TABLE 12
Comparability data from representative GPOR OPC1 and LCTOPC1 R&D Runs - non-targeted/impurities
cell population gene profile by qPCR (relative to GPOR OPC1 M08)

| | | | | | | | LCTOPC1 | | |
|---|---|---|---|---|---|---|---|---|---|
| Cell | Current | Proposed | | GPOR OPC1 | | | RD | RD | RD |
| population | Marker | Marker | Justification | M08¹ | M22¹ | M25¹ | 11A2 | 11C | 12D1 |
| Endoderm | AFP | AFP | Known endoderm genes | 1.00 | 1.35 | 1.11 | ND | ND | ND |
| | | FOXA2 | | ND | ND | ND | ND | ND | ND |
| | | SOX17 | | ND | ND | ND | ND | ND | ND |
| Mesenchymal | OLR1 | OLR1 | OLR1 induces epithelial- | 1.00 | 1.31 | 1.77 | ND | ND | ND |
| | | | mesenchymal transition | | | | | | |

*ND—Not Detected
¹Clinical batch

TABLE 13
Comparability data from representative GPOR OPC1 and LCT OPC1 R&D Runs -
in vitro function as determined by decorin secretion and migration assays

| | | | | | LCTOPC1 | | |
|---|---|---|---|---|---|---|---|
| | | GPOR OPC1 | | | RD | RD | RD |
| Test | Justification | M08¹ | M22¹ | M25¹ | 11A2 | 11C | 12D1 |
| Decorin Secretion (ng/mL) | Attenuates scar tissue, blocks | 25.6 | 27.61 | 23.75 | 36.67 | 29.12 | 29.78 |
| | cavitation, promotes wound healing | | | | | | |
| Cells Migration (% | Potential mode of action; promote | 36 | 47 | 33 | 49 | 44 | 35 |
| migrated, PDGF-bb induced) | healing in tissue | | | | | | |

¹Clinical batch

### Review of Ectopic Tissue Formation Assessment as a Release Criterion for OPC1 Drug Product

OPC1 program historical approach to address ectopic tissue (cysts) formation risks. Toxicity studies performed and provided as part of the initial OPC1 IND submission revealed ectopic tissues formation of cystic structures and granular hyperplasia (AGS) in animals that received certain OPC1 batches. Substantial lot-to-lot variation and FDA concerns that the proposed release criteria are not sufficient to predict the safety of the intended clinical lots led to FDA's request to conduct 9-months toxicology study for each intended clinical lot as a release requirement. An in vitro cyst assay was presented as a research tool that was used to screen OPC1 lots. The hypothesis stated that lots that have a strong propensity to form cysts in vitro could be readily identified, and that all of the in vitro cyst positive lots would show cyst formation in vivo. Additionally, it was stated that steps are being taken to refine the assay by improving cyst identification through more consistent staining and evaluation of in vitro cyst size as a predictor for in vivo cyst formation. Further work performed on in vitro cyst assay revealed the following conclusions: despite several years of development efforts, the cyst assay does not meet the basic requirements for QC release method; there is no linear correlation or an accurate prediction between in vitro cyst formation and in vivo cyst frequency.

Review of OPC1 safety status based on GPOR data and preliminary results from comparability testing, correlation between pass/fail GPOR batches, and new/developed markers. We have been continuously working on the improvement of the OPC1 manufacturing process, including new in-process controls, release, and potency testing for OPC1 product. The rationale for process improvement and comparability plan for manufactured material was presented in the previous sections of this Example. As part of development efforts, we have performed a comprehensive analysis and retesting of the relevant GPOR OPC1 GMP batches using the improved product characterization panel based on newly developed methods. This analysis revealed certain correlations between the available data from GLP tox studies of Geron GMP batches to the expression level of purity and impurities/non targeted cell population specific markers. The retesting results and analysis show correlation between impurities and batch failure in vivo, indicating that LCTOPC1, characterized by significant lower levels of impurities and high purity profile, has a greatly reduced likelihood of cyst formation in vivo than does GPOR OPC1.

### SUMMARY

Preliminary comparability data of representative GPOR and LCTOPC1 batches demonstrate the following: GPOR OPC1 and R&D LCTOPC1 demonstrate similar OPC1 identity/purity profile profiles, except for the PDGFR-α biomarker which is higher in R&D LCTOPC1 and may result from the improved OPC1 manufacturing process (directed differentiation); R&D LCTOPC1 has lower levels of impurities from epithelial, astrocytes and neuronal non-targeted cells, compared to GPOR OPC1; both LCTOPC1 and GPOR OPC1 have very rarely detectable residual hESC (detected by % TRA-1-60+/Oct4+), however, GPOR OPC1 has a higher percentage of multipotent cells compared to LCTOPC1 as demonstrated by populations of cells exhibiting TRA-1-60+/Oct4− and CD49f+; LCTOPC1 has lower levels of endoderm and mesenchymal non-targeted cells gene expression, compared to GPOR OPC1; LCTOPC1 and GPOR OPC1demonstrate similar decorin secretion and migration capacity.

Conclusions. The data accumulated to date show that LCTOPC1 drug product manufactured with an improved process, generated from a defined cell banking system by a highly-reproducible and tightly-controlled differentiation process and monitored with updated analytical methods, presents similar essential quality attributes to GPOR OPC1, but benefits from overall higher expression of purity markers and lower expression of an impurity/non-targeted population markers. Thus, potentially increasing the safety profile for LCTOPC1 drug product.

### Example 3: Proof of Concept Study

The purpose of this study is to provide experimental data for OPC1 myelinization and maturation proof of concept studies.

LCT OPC1 cells were cultured on Matrigel (MG) 1:30 in 96 vision plates in different media components as listed in Table 14. Each culture condition consists a “system”. The cells were cultured in GPM/E for the first 5-6 days of culturing, then for the maturation systems, media was replaced according to the steps listed in Table 14. System #2 showed morphology change to Oligodendrocyte like cells, and MBP expression by immunostaining.

TABLE 14
Study Design

| | | Media up to | | | | |
|---|---|---|---|---|---|---|
| | | days 5-6 | Media at | Media at | | |
| | | (culturing + | days 5-8 | days 7-10 | Media for | |
| | | 2 media | (3rd media | (4th media | the rest of | |
| Cell type | Substrate | changes) | change) | change) | the assay | System# |
| OPC1-RD | Vision | GPM/E | GPM/E | GPM/E | GPM/E | 1 - Ctrl |
| | 96-well | (GPM + | (GPM + | (GPM + | (GPM + | |
| | plate | hEGF) | hEGF) | hEGF) | hEGF) | |
| | (type#1) | | 50% GPM/E + | GPM/E + | GPM/E + | 2 |
| | coated | | 50% GPM/E + | N-2 with | N-2 with | |
| | with | | N-2 with | 500 uM db- | 500 uM db- | |
| | GFR-MG | | 500 uM db- | cAMP and | cAMP and | |
| | (1:30) | | cAMP and | 500 uM | no IBMX | |
| | | | no IBMX | IBMX | | |

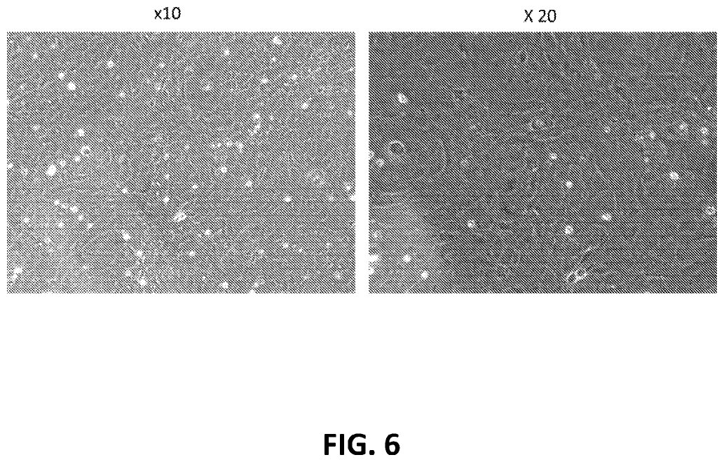

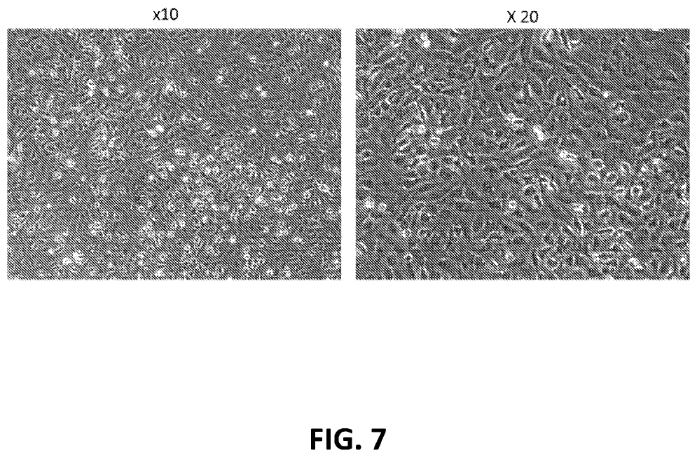

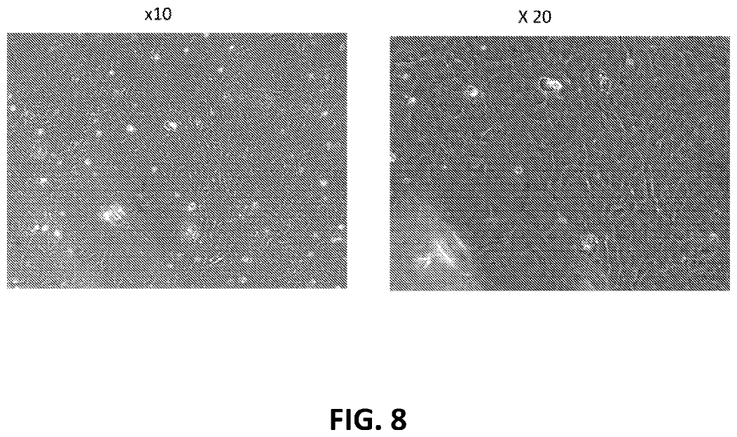

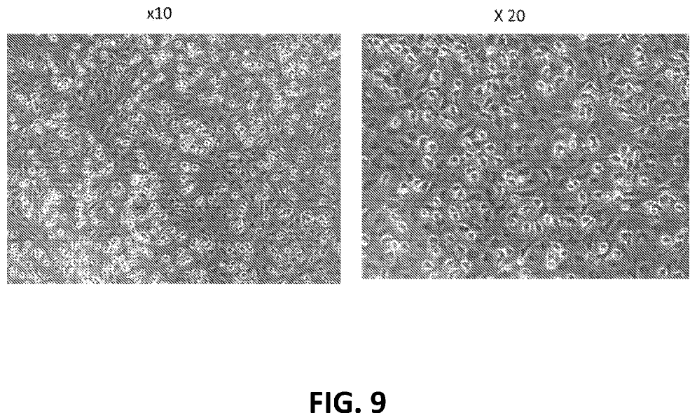

FIGS. 6-9 show OPC morphology for a different systems. FIG. 6 shows control system #1, GPM/E at day 10 (time=0, day 7). OPC morphology is typical for DCN ELISA potency assay. FIG. 7 shows OPC morphology for system #2, 50% GPM/E and 50% N2.1 (without IBMX) on day 3 after treatment. Cells show a morphological change compared to control. FIG. 8 shows control system #1 at day 12, and OPC morphology typical for DCN ELISA potency assay. FIG. 9 shows system #2, 100% N2.1 (with IBMX for 2 days) on day 5 after treatment. Cells show a morphological change

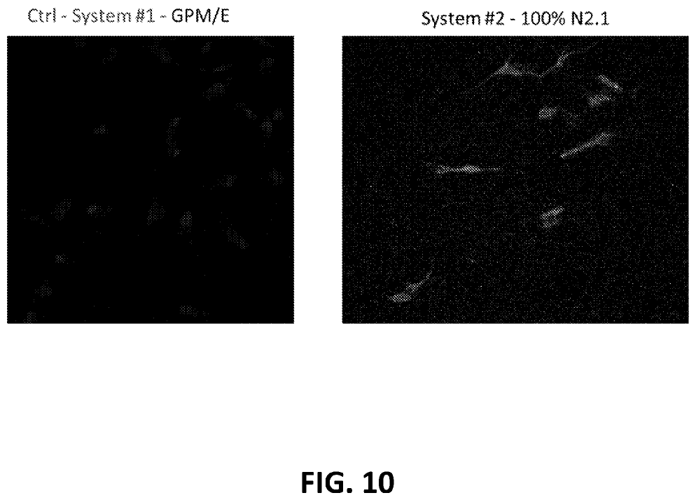

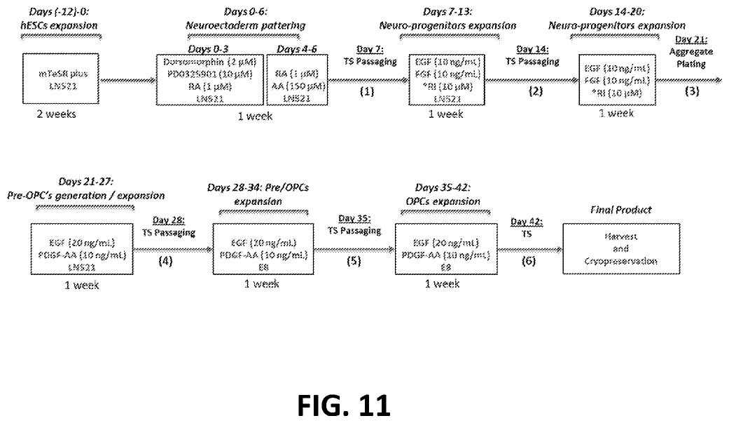

FIG. 10 shows confocal immunostaining for Hoechst and MBP on day 13 (fixed cells). Control system #1 in GPM/E is shown on the left, and system #2 with 100% N2.1 is shown on the right.

### Example 4: OPC Differentiation Protocol

TABLE 15
Volume of stock solution to add per ml of GPM to reach desired final concentration

| | | Small | Thawing | Stock | | Final | | Volume of stock | |
|---|---|---|---|---|---|---|---|---|---|
| Day in process | | molecules | conditions | concentration | | concentration | | solution/ml of GPM | |
| Days | 0-3 | Dorsomorphin | RT/25° C. water bath | 2 | mM | 2 | μM | 1 | μl/ml |
| | | | or at 37° C. incubator | | | | | | |
| | | | for up to 5 min. protect | | | | | | |
| | | | from light | | | | | | |
| | | PD0325901 | RT/25° C. water bath | 10 | mM | 10 | μM | 1 | μl/ml |
| | | Retinoic Acid | RT or at 37° C. | 20 | mM | 1 | μM | 0.05 | μl/ml |
| | | | incubator for up | | | | | | |
| | | | to 20 min. protect | | | | | | |
| | | | from light | | | | | | |
| Days | 4-6 | Retinoic Acid | RT or at 37° C. | 20 | mM | 1 | μM | 0.05 | μl/ml |
| | | | incubator for up | | | | | | |
| | | | to 20 min. protect | | | | | | |
| | | | from light | | | | | | |
| | | Ascorbic Acid | RT | 150 | mM | 150 | μM | 1 | μl/ml |
| Day 7, 14 | | RI | RT | 10 | mM | 10 | μM | 1 | μl/ml |
| | | EGF | RT | 20 | μg/ml | 10 | ng/ml | 0.5 | μl/ml |
| | | hs-FGF | RT | 10 | μg/ml | 10 | ng/ml | 1 | μl/ml |
| | | EGF | RT | 20 | μg/ml | 10 | ng/ml | 0.5 | μl/ml |
| Days 9-20 | | hs-FGF | RT | 10 | μg/ml | 10 | ng/ml | 1 | μl/ml |
| (excluding | | | | | | | | | |
| day 14) | | | | | | | | | |
| Days | 21-42 | EGF | RT | 20 | μg/ml | 20 | ng/ml | 1 | μl/ml |

| | | | | | | | |
|---|---|---|---|---|---|---|---|
| PDGF-AA | RT | 100 | μg/ml | 10 | ng/ml | 0.1 | μl/ml |

The purpose of this example is to demonstrate establishment of GMP conditions for differentiation of hESCs into OPCs.

Day 0-culture evaluation. Day 0 IPC: Before starting stage II, perform hESCs colonies evaluation. Differentiation can initiate only when the following criteria are met: confluency in culture is >95%; % of differentiation in culture is <5%; % of tightly packed colonies is >50%; and lactate concentration in media is >17.40 mM.

Media replacement, days 0-6: Media should be replaced every 24 hr±4 hr. On day 0 and 4, observe and photograph the culture using 5×, and 10× magnifications. On days 3 and 4, measure lactate. Thaw small molecules according to Table 15 and warm the required GPM volume. Add the GFs volume to the warmed GPM according to Table 15. Replace medium. Starting from day 4, GFs are changed according to Table 15.

Harvesting, day 7: If applicable, one day up to a week before harvesting determine and coat with 10 μg/ml rh LN521 the required number of flasks. Observe and photograph the culture using 5×, and 10× objectives. Measure lactate level. Divide 2 ml of CM into two cryovials (1 ml in each), store at −80° C. Thaw GFs according to Table 15 and warm the required GPM volume (for final solutions of GPM+GFs and GPM w/o GFs). Add the GFs volume to the warmed GPM according to Table 15.

Harvesting Protcol: Aspirate the medium. Add 3 ml PBS (−) to each T25 flask. Tilt the flask 2-3 times and incubate 1 minute at RT. Use timer. Aspirate and add 1 ml/T25 TrypLE Select. Note: open a new bottle of TS. Incubate for 6 min, at 37° C. and 5% CO₂. Use timer. Tap the flask very firmly on one side, tilt the flask to wash the cells and tap very firmly on the other side of the flask. Add 5 ml of GPM w/o GFs. Add the medium directly on the cells, wash the surface 1-2 times gently. Collect the cells into a conical tube. Add 5 ml/T25 GPM w/o GFs and rinse 2-3 times gently. Collect the cells. Centrifuge: 200 g, 5 min, RT. Use timer. Optional: Take images using 2.5× objectives of each treated flask after quenching, to record the % of the remaining cells. Aspirate the supernatant, tap the pellet firmly and re-suspend gently with 5 ml GPM+GFS per T25 flask harvested using 5 ml pipette. Be careful not to over pipette.

Cell Counting Protocol: Prepare two Marked Eppendorf tubes and transfer ˜100 μl of the cell suspension into each separate Eppendorf tube. For ×5 dilution: Prepare two additional Eppendorf tubes with 200 μl of GPM+GFs, and transfer to each medium containing tube, 50 μl of the cell suspension, prepared in section 0 to a final volume of 250 μl. Mix well. Count each diluted Eppendorf tube using the NC-200 cell counter.

Cell Seeding Protocol: According to Table 16, calculate the required medium volume and number of cells for seeding (Density of 2.67×10⁴ live cells/cm²).

TABLE 16
Volume and # of cells for seeding

| Flask | # of live cells/flask | Total volume/flask |
|---|---|---|
| T225 | 6 × 10⁶ | 90 ml |

If applicable, retrieve the LN521 coated flasks from the refrigerator. For LN521 coated flasks: Add 6 ml GPM+GFs to each T225 LN521 coated flask. Incubate the flasks in RT for 1 min. Use a timer. Aspirate the coating solution and the GPM+GFs medium from the flasks. Wash with 24 ml PBS(+)/T225. Add the calculated GPM+GFs into the flask. Pipette gently to mix the cell suspension and seed in the calculated cells volume. Transfer the vessels to the incubator, gently agitate the vessel to evenly distribute the cells over the surface of the vessel. IPC Day7: Cryopreserve cells for samples.

Media replacement, days 9-13: On days: 9, 11 and 13, observe the culture, photograph using 2.5×, 5×, and 10× objectives and measure lactate. Thaw GFs according to Table 15. Determine the GPM volume needed and warm at 37° C. water bath. Add GFs to the GPM according to Table 15. Aspirate the medium and add 90 ml GPM+GFs medium to each T225 flask.

Harvesting, day 14: PBS-wheel preparation: on day 13, label and pre-load the PBS-wheel with 70 ml of the GPM medium, tilt the PBS and wash the walls of the wheel. Incubate overnight in a humidified CO₂ incubator at 37° C. and 5% CO₂ at 35 RPM. On day 14, observe the culture, photograph using 2.5×, 5×, and 10× objectives. Measure Lactate. Divide 2 ml of CM into two cryovials (1 ml in each), store at −80° C. Thaw GFs according to Table 15 and warm the required GPM volume (for final solutions of GPM+GFs and GPM w/o GFs). Add the GFs volume to the warmed GPM according to Table 15.

Harvesting: Aspirate the medium. Add 24 ml PBS(−) to each T225 flask. Tilt the flask 2-3 times and incubate 1 minute at RT. Aspirate and add 9 ml/T225 TrypLE Select. Incubate for 8 min, at 37° C. and 5% CO₂. Tap the flask firmly on one side, tilt the flask to wash the cells and tap firmly on the other side of the flask. Add 18 ml of GPM w/o GFs. Add the medium directly on the cells. Collect the cells into a conical tube. Add 18 ml/T225 GPM w/o GFs and rinse 2-3 times gently. Collect the cells. Centrifuge: 170-180 g, 5 min, RT. Optional: Take images using 2.5× objectives of each treated flask after quenching, to record the % of the remaining cells. Aspirate the supernatant, tap the pellet firmly and re-suspend with 30 ml (first resuspend with 5 ml in 5 ml pipette and then add 25 ml) GPM+GFS per T225 flask harvested. Be careful not to over pipette.

Cell counting: Prepare two Marked Eppendorf tubes and transfer ˜100 μl of the cell suspension into each separate Eppendorf tube. For X4 dilution: Prepare two additional Eppendorf tubes with 150 μl of GPM+GFs, and transfer to each medium containing tube, 50 μl of the cell suspension to a final volume of 200 μ1. Mix well. Count each diluted Eppendorf tube using the NC-200 cell counter.

Cell Seeding: According to Table 17, calculate the required medium volume and number of cells for seeding (128×10⁶ live cells/0.1PBS wheel).

TABLE 17
Volume and # of cells for seeding

| Vessel | # of live cells/wheel | Total volume/wheel |
|---|---|---|
| 0.1PBS wheel | 128 × 10⁶ | 70 ml |

Aspirate the pre-loaded medium from the wheels. Add the calculated GPM+GFs into the flask. Pipette gently to mix the cell suspension and seed in the calculated cells volume. Place the PBS wheel vessels on the PBSmini in a humidified CO₂ incubator at 37° C. and 5%. Set the PBSmini speed to 35 RPM for 24hr±4. IPC Day14: Cryopreserve cells.

Day 15: Increase the PBSmini speed to 45 RPM. Turn on the light of the PBSmini and examine carefully that clots are not seen while the PBS-wheel is still rotating. Only if clots were observed, quickly remove them to avoid sedimentation. if not necessary, do not remove the PBS-wheel from the PBSmini.

Medium Replacement: days 16, 18, 20: On days: 16, 18 and 20, observe the culture, photograph using 2.5× and 5× objectives and measure Lactate. Thaw GFs according to Table 15. Determine the GPM volume needed and warm at 37° C. water bath. Add GFs to the GPM according to Table 15. Remove the PBS-Wheel from the PBSmini and place it on a rack in the incubator for the indicated settling time per day, as in Table 18.

TABLE 18
Settling time per day

| | Day | | |
|---|---|---|---|
| | 16 | 18 | 20 |
| Time (min) | 10 | 5 | 5 |

Inspect the settling before media replacement. If aggregates are not settled, increase the settling time for additional 5 min. Using a 10 ml pipette, remove 80% (56 ml) of the volume in the PBS-Wheel, keep it for lactate measurement. Add 56 ml (the same volume of GPM+GFs as removed in the section above) to the side of the PBS-Wheel. Inspect the PBS-wheel for clots formation, if clots are observed, remove them, as the examination and removal of clots is crucial for the process. Return the PBS-Wheel to the PBSmini in the humidified CO₂ incubator at 37° C. and 5% CO₂, 45 RPM. Measure Lactate.

Flattening of Aggregates, Day 21: One day up to a week before harvesting determine and coat with appropriate coating solution (10 μg/ml rhLN521 or iMatrix-511-E8) the number of T75/T150 flasks. To determine the vessel for seeding, measure lactate approximately 24 hours after media change. If lactate concentration is ≤15.00 mM seed aggregates in one T75. Otherwise, seed aggregates in one T150. Divide 2 ml of CM into two cryovials (1 ml in each), store at −80° C. Observe the culture and photograph the aggregates. Thaw GFs according to Table 15 and warm the required GPM volume (for final solutions of GPM+GFs). Add the GFs volume to the warmed GPM according to Table 15.

Preparation of coated vessels for seeding: Retrieve the coated flasks from the refrigerator. Label each flask. Add 2/4 ml GPM+GFs to each T75/T150 coated flask. Incubate the flasks in RT for 1 min. Use a timer. Aspirate the coating solution and the GPM+GFs medium from the flasks. Only for LN521 coated flasks—wash with 10/20 ml PBS (+) for each T75/T150 coated vessel. Add 20/40 ml GPM+GFs to the T75/T150. Transfer the vessels to a humidified CO₂ incubator at 37° C. and 5% CO₂ until plating. Using a 25 ml pipette transfer 35 ml of the PBS-Wheel content into a 50 ml conical tube. Let the aggregates settle for 4 min. Aspirate manually using a 25 ml pipette and remove 30 ml of GPM to a separate 50 ml tube. Repeat steps (25 ml pipette transfer 35 ml of the PBS-Wheel content into a 50m1 conical tube, let the aggregates settle for 4 min), to transfer the rest of the aggregates from the PBS-Wheel into the same 50 ml conical tube labeled “seeding aggregates”. During aggregates settling, using a 2 ml pipette, collect the rest of the aggregates left in the PBS-wheel and transfer slowly into the bottom of the “seeding aggregates” tube. While aggregates are settling in the 50 ml tube, add 20 ml GPM+GFs into the PBS-wheel. Repeat steps (25 ml pipette transfer 35 ml of the PBS-Wheel content into a 50 ml conical tube, let the aggregates settle for 4 min, transfer the rest of the aggregates from the PBS-Wheel into the same 50 ml conical tube labeled “seeding aggregates”, during aggregates settling, using a 2 ml pipette, collect the rest of the aggregates left in the PBS-wheel and transfer slowly into the bottom of the “seeding aggregates” tube, while aggregates are settling in the 50 ml tube, add 20 ml GPM+GFs into the PBS-wheel) once more. Collect the wash from the PBS-wheel into the 50 ml tube. Tilt the PBS-wheel, and with a 2 ml pipette collect the remaining medium from the PBS-wheel. Centrifuge the 50 ml conical tube: 140G, 3 min., RT. Transfer the prepared vessels into the BSC. Verify that the upper surface of the flask is clear. If necessary, wash the surface with the medium before seeding. Aspirate gently the supernatant. Using a 25 ml pipette, re-suspend the pellet (do not tap before resuspension), with 5/10 ml GPM+GFs per seeding of T75/T150. Measure the suspension volume. Seed the total volume to the designated vessel quickly (keep the flask vertically to avoid attachment of aggregates non-homogenously). Using a 25 ml pipette wash the tube with the extra volume (to complete 30/60 ml per 75/150 cm² seeded, respectively) and add to the vessel. Incubate the flasks in a humidified incubator at 37° C. and 5% CO₂. In the incubator make sure the aggregates are distributed uniformly in the flask.

Medium replacement days 23, 25, 27: On days: 23, 25 and 27, observe the culture, photograph using 5×, and 10× objectives and measure Lactate. Thaw GFs according to Table 15. Determine the GPM volume needed and warm at 37° C. water bath. Add GFs to the GPM according to Table 15. Aspirate the medium and add 30/60 ml GPM+GFs medium to each T75/T150 flask.

Day 28: If applicable, one day up to a week before harvesting determine and coat with 10 μg/ml rhLN521 the required number of flasks. Observe and photograph the culture using 5×, and 10× objectives. Measure lactate level. Divide 2 ml of CM into two cryovials (1 ml in each), store at −80° C. Thaw GFs according to Table 15 and warm the required GPM volume (for final solutions of GPM+GFs and GPM w/o GFs). Add the GFs volume to the warmed GPM according to Table 15. Harvesting: Aspirate the medium. Add 8/16 ml PBS(−) to each T75/T150 flask. Tilt the flask 2-3 times and incubate 1 minute at RT. Aspirate and add 3/6 ml per T75/T150 TrypLE Select. Incubate for 7 min, 37° C. and 5% CO₂. Tap the flask gently on one side, tilt the flask to wash the cells and tap gently on the other side of the flask. Using 25 ml pipette, add gently 6/12 ml of GPM w/o GFs on the cells' surface. Collect the cells into a conical tube. Do not wash cell surface. Centrifuge: 260-270 g, 10 min, RT. Aspirate the supernatant, tap the pellet gently until the pellet is detached from the tube and re-suspend with 15/30 ml GPM+GFS per T75/T150 flask harvested. First, with a 25 ml pipet, resuspend with 5 ml, pipet up and down 2-3 times and then add the remaining volume using 25 ml pipette. Be careful not to over pipette.

Cell counting: Prepare two Marked Eppendorf tubes and transfer ˜150 μl of the cell suspension into each separate Eppendorf tube avoid transferring aggregates for the counting samples. For X2 dilution: Prepare two additional Eppendorf tubes with 100 μl of GPM+GFs, and transfer to each medium containing tube, 100 μl of the cell suspension to a final volume of 200 Mix well. Count each diluted Eppendorf tube using the NC-200 cell counter.

Cell Seeding: According to Table 16, calculate the required medium volume and number of cells for seeding (Density of 4.0×10⁴ live cells/cm²).

TABLE 19
Volume and # of cells for seeding

| Flask | # of live cells/flask | Total volume/flask |
|---|---|---|
| T225 | 9 × 10⁶ | 90 ml |

If applicable, retrieve the LN521 coated flasks from the refrigerator. Label each flask. For LN521 coated flasks: Add 6 ml GPM+GFs to each T225 LN521 coated flask. Incubate the flasks in RT for 1 min. Use a timer. Aspirate the coating solution and the GPM+GFs medium from the flasks. Wash with 24 ml PBS(+)/T225. Add the calculated GPM+GFs into the flask. If applicable, prepare medium+E8 pool. Pipette gently to mix the cell suspension and seed in the calculated cells volume. Transfer the flasks to the incubator, gently agitate the flasks to evenly distribute the cells over the surface of the vessel.

IPC Day 28: Cryopreserve cells. Medium replacement days 30, 32, 34: On days: 30, 32 and 34, observe the culture, photograph using 5×, and 10× objectives and measure Lactate. Thaw GFs according to Table 15. Determine the GPM volume needed and warm at 37° C. water bath. Add GFs to the GPM according to Table 15. Aspirate the medium and add 90 ml GPM+GFs medium to each T225 flask.

Day 35: On day 35, observe the culture, photograph using 5×, and 10× objectives. Measure Lactate. Divide 2 ml of CM into two cryovials (1 ml in each), store at −80° C. Thaw GFs according to Table 15 and warm the required GPM volume (for final solutions of GPM+GFs and GPM w/o GFs). If cells are not seeded for further culturing, do not thaw GFs and do not prepare GPM+GFs, only GPM w/o GFs is required. Add the GFs volume to the warmed GPM according to Table 15.

Harvesting: Aspirate the medium. Add 24 ml PBS(−) to each T225 flask. Tilt the vessel 2-3 times and incubate 1 minute at RT. Aspirate and add 9 ml/T225 TrypLE Select. Incubate for 7 min at 37° C. and 5% CO₂. Tap the vessel gently on one side, tilt the flask to wash the cells and tap gently on the other side of the vessel. Add 18 ml of GPM w/o GFs. Add the medium directly on the cells. Collect the cells into a conical tube. Add 18 ml/T225 GPM w/o GFs and rinse 2-3 times gently. Collect the cells. If cells are only harvested without seeding, proceed to cell counting. If cells are seeded for further culturing, centrifuge: 260-270 g, 10 min, RT. Aspirate the supernatant, tap the pellet firmly and re-suspend with 30 ml GPM+GFs per T225 vessel harvested. First, resuspend with 5 ml, pipet up and down 2-3 times and then add the remaining volume using 25 ml pipette. Be careful not to over pipette.

Cell counting: Prepare two additional Eppendorf tubes with and transfer to each tube, 200 μl of the cell suspension. Count each tube using the NC-200 cell counter.

Cell Seeding (if applicable): According to Table 16, calculate the required medium volume and number of cells for seeding (Density of 4.0×10⁴ live cells/cm²).

TABLE 20
Volume and # of cells for seeding

| Flask | # of live cells/flask | Total volume/flask |
|---|---|---|
| T225 | 9 × 10⁶ | 90 ml |

If applicable, retrieve the LN521 coated flasks from the refrigerator. Label each flask. For LN521 coated flasks: Add 6 ml GPM+GFs to each T225 LN521 coated flask. Incubate the flasks in RT for 1 min. Use a timer. Aspirate the coating solution and the GPM+GFs medium from the flasks. Add the calculated GPM+GFs into the flask. If applicable, prepare medium+E8 pool. Pipette gently to mix the cell suspension and seed in the calculated cells volume. Transfer the flasks to the incubator, gently agitate the flasks to evenly distribute the cells over the surface of the flasks. IPC Day35: Cryopreserve cells.

Medium replacement days 37, 39, 41: On days: 37, 39 and 41, observe the culture, photograph using 5×, and 10× objectives and measure Lactate. Thaw GFs according to Table 15. Determine the GPM volume needed and warm at 37° C. water bath. Add GFs to the GPM according to Table 15. Aspirate the medium and add 90 ml GPM+GFs medium to each T225 flask.

Day 42: On day 42, observe the culture, photograph using 5×, and 10× objectives. Measure Lactate. Divide 2 ml of CM into two cryovials (1 ml in each), store at −80° C. Warm the required GPM volume.

Harvesting: Aspirate the medium. Add 24 ml PBS(−) to each T225 flask. Tilt the flask 2-3 times and incubate 1 minute at RT. Aspirate and add 9 ml/T225 TrypLE Select. Incubate for 7 min at 37° C. and 5% CO₂. Tap the flask gently on one side, tilt the flask to wash the cells and tap gently on the other side of the flask. Add 18 ml of GPM w/o GFs. NOTE: Add the medium directly on the cells. Collect the cells into a conical tube. Add 18 ml/T225 GPM w/o GFs and rinse 2-3 times gently. Cell counting: Prepare two additional Eppendorf tubes with and transfer to each tube, 200 μl of the cell suspension. Count each tube using the NC-200 cell counter. IPC Day42: Cryopreserve cells.

### Example 5: Production of Large Scale OPC Batch

The purpose of this example is to demonstrate establishment of large scale conditions for generation of OPCs.

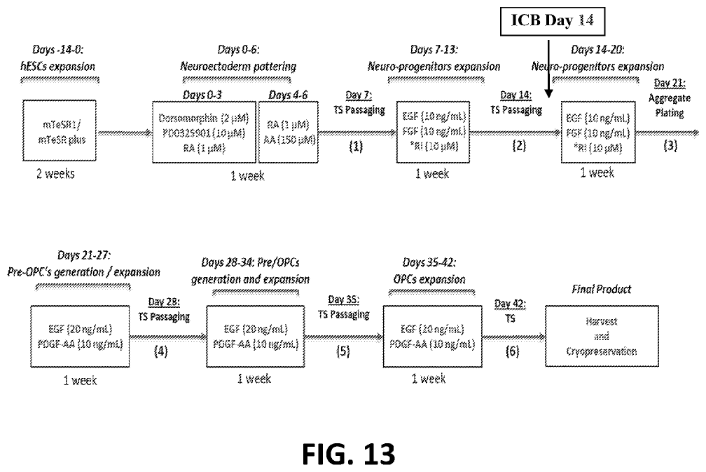

Experimental Procedure: FIG. 13 shows the steps of the OPC differentiation protocol.

hESCs culturing (stage 1)—H1 hESCs (PILOT-LCT-H1-002) were cultured for 3 passages according to ERIs #ERI-H1-01 and #ERI-H1-02 on LN521 coated vessels. At the end of p30+6+4+2, cells were seeded for 4 days for OPC differentiation initiation.

OPC Differentiation—On day 4 of p30+6+4+3 of stage 1, OPC differentiation was initiated in six T25 vessels according to FIG. 13. (Day 0 to differentiation protocol).

Days 0-3—Culturing as above. GPM medium supplemented with 2 μM Dorsomorphin, 10 μM PD0325901, and 1 μM RA, was changed daily.

Days 4-6—Culturing according to the protocol in Example 5. GPM medium supplemented with 150 μM AA, and 1 μM RA, was changed daily.

Day 7—Cells were harvested according to the protocol in Example 5 with TS. 11 T225 vessels were seeded in GPM supplemented with 10 ng/ml EGF, 10 ng/ml hs-FGF, and 10 μM RI at 26,667 live cells/cm² on LN521 coated vessels.

Day 9-13—Culturing according to the protocol in Example 5. GPM medium supplemented with 10 ng/ml EGF, and 10 ng/ml hs-FGF was changed every 2 days.

Day 14—Cells were harvested according to the protocol in Example 5 with TS and 12 PBS wheels seeded (for #20-EROPCRD-02 ongoing run) for aggregate formation in GPM supplemented with 10 ng/ml EGF, 10 ng/ml hs-FGF, and 10 μM RI at 128×10⁶ live cells/PBS wheel. In addition, all other cells were cryopreserved as ICB in CS10.

Day 14 thawing—For ICB thawing, cryopreserved cells were thawed, counted, and seeded in PBS wheels according to #ER-ICBRD-02 study design. 128×10⁶ live cells/PBS wheel were seeded in 6 vessels according to the protocol in Example 5 and 105×10⁶ live cells/PBS wheel were seeded in one vessel.

Day 16-20—Culturing according to the protocol in Example 5. 80% of GPM medium supplemented with 10 ng/ml EGF, and 10 ng/ml hs-FGF was changed every 2 days.

Day 21—Aggregates were flattened on day 21 and seeded in GPM supplemented with 20 ng/ml EGF, and 10 ng/ml PDGF-AA on 7 T75 LN521 coated vessels.

Days 23-27—Culturing as above. GPM medium supplemented with 20 ng/ml EGF, and 10 ng/ml PDGF-AA was changed every 2 days.

Day 28—Cells were harvested with TS and seeded in GPM supplemented with 10 ng/ml EGF, and 10 ng/ml PDGF-AA at 40,000 live cells/cm² on 36 T225 E8 direct coated vessels.

Days 29-34—Culturing as above. GPM medium supplemented with 20 ng/ml EGF, and 10 ng/ml PDGF-AA was changed every 2 days.

Day 35—Cells were harvested with TS and seeded in GPM supplemented with 10 ng/ml EGF, and 10 ng/ml PDGF-AA at 40,000 live cells/cm² on 45 Cell-factories E8 direct coated vessels. Deviation in CO2 levels in the incubator on day 35 was reported. Since no difference appeared between vessel affected from the deviation and all others, by morphology and lactate levels evaluation, it was decided to proceed with all the vessels, as the deviation involves minor risk for the cells.

Days 36-41—Culturing as above. GPM medium supplemented with 20 ng/ml EGF, and 10 ng/ml PDGF-AA was changed every 2 days.

Day 42—Harvesting and cryopreservation of cells with TS in TAI formulation, according to #ERI-OPC-07.

QC tests—Flow cytometry—Analysis of markers by FACS was performed on cryopreserved cells on days 7, 14, 28, 35, and 42 according to Table 21.

TABLE 21
FACS staining

| Category | Subcategory | Marker | Day 7 | Day 14 | Day 28 | Day 35 | Day 42 |
|---|---|---|---|---|---|---|---|
| Purity/ | OPC | PDGFRα | ✓ | ✓ | ✓ | ✓ | ✓ |
| Identity | | PDGFRβ | | | | ✓ | ✓ |
| | | NG2 | ✓ | ✓ | ✓ | ✓ | ✓ |
| | Pre-OPC | A2B5 | ✓ | ✓ | ✓ | ✓ | ✓ |
| | | GD3 | ✓ | ✓ | ✓ | ✓ | ✓ |

| Impurities/ | TRA-1-60 | ✓ | ✓ | | ✓ | ✓ |
|---|---|---|---|---|---|---|
| Non-targeted | Claudin 6 | ✓ | ✓ | ✓ | ✓ | ✓ |
| population | EpCAM | ✓ | ✓ | ✓ | ✓ | ✓ |
| | Keratin 7 | | | | ✓ | ✓ |
| | CD49f | | | | ✓ | ✓ |
| | Beta Tubulin | | | | ✓ | ✓ |
| | III | | | | | |
| | GFAP | | | | ✓ | ✓ |

Batch Release Acceptance criteria—Proposed acceptance criteria for batch release testing of day 42 cells are presented in Table 22.

TABLE 22
Batch Release proposed acceptance criteria.

| | | | Proposed |
|---|---|---|---|
| Test | Method | Target tested | Criteria |
| Viability | NC-200 | % of viable | ≥70% |
| | | population | |
| Strength | NC-200 | Live cells/ml | 0.75-1.25 × 10⁸ |
| | | | Live cells/ml |
| Sterility | <USP71> | Turbidity | No growth |
| Mycoplasma | <USP63> | Mycoplasma (CFU) | No growth |
| Endotoxin | <USP85> | Endotoxin (EU/ml) | <2 EU/ml |
| OPC | Flow | % GD3⁺ | ≥80% |
| | Cytometry | % NG2⁺ | ≥80% |
| | | % PDGFRa⁺ | ≥90% |
| | | % PDGFRβ⁺ | ≥90% |
| Epithelial | Flow | % Keratin 7⁺ | ≤2% |
| | Cytometry | % of Claudin-6⁺ | ≤2% |
| | | % of EpCAM⁺ | ≤2% |
| | | % CD49f | ≤10% |
| hESCs Residual | | % TRA-1-60⁺ | ≤1% |
| | | % TRA-1-60⁺/ | FIO |
| | | Oct-4⁺ | |
| Mesoderm | | % GFAP | ≤2% |
| Astrocytes | | % CXCR4⁺/CD56⁺ | FIO |
| Neuronal Phenotype | | % b-Tubulin III⁺ | FIO |
| Mesenchymal | qPCR | OLR1 | FIO |
| Endoderm | | FOXA2, SOX17, | FIO |
| | | AFP | |
| Decorin Secretion | ELISA | Decorin (ng/ml) | ≥25 ng/ml |
| Cell Migration | NC-200 | % Migrated | ≥30% |
| | | (PDGF-induced) | (+PDGFββ) |

Results: Passaging parameters—All passaging parameters regards #20-EROPCRD-02 and #ER-ICBRD-02 are presented in Table 23 and at Table 24. At each passage, yield and PDL values were calculated according to the following formulas:

$$a . {Yield}_{days 7 , 14 , 35 , 42} = \frac{Harvesting density ( on day x ) [ live cells per {cm}^{2} ]}{Seeding density ( last passage ) [ live cells per {cm}^{2} ]} \\ b . {Yield}_{day 28} = \frac{Harvested cells ( on day 28 )}{Seeded cells ( on day 11 )} \\ c . PDL = {log}_{2} ( Yield )$$

TABLE 23
Passaging parameters days 0-28

| | | Day 7 | | | | | Day 14 | | |
|---|---|---|---|---|---|---|---|---|---|
| | | | | Harvesting | | | | | Harvesting |
| | Day 0 | | | density | | | | | density |
| | Lactate | Lactate | % | (live | | | Lactate | % | (live |
| Run | (mM) | (mM) | viability | cells/cm²) | Yield | PDL | (mM) | viability | cells/cm²) |
| Ongoing | 18.51 | 20.89 | 73 | 1.34 × 10⁶ | 105.67 | 6.7 | 12.83 | 99 | 1.12 × 10⁶ |
| ICB | | | | | | | | | |

| | | | | Day 28 | | | | | |
|---|---|---|---|---|---|---|---|---|---|
| | | | | | | | Harvesting | | |
| | | | Day 21 | | | Estimated | density | | |
| | Day 14 | | Lactate | Lactate | % | cell diameter | (live | | |
| Run | Yield | PDL | (mM) | (mM) | viability | (μm) | cells/cm²) | Yield | PDL |
| Ongoing | 42.10 | 5.4 | 6.57 | Aborted | | | | | |
| ICB | | | 7.58 | 8.46 | 85 | 11.3 | 7.14 × 10⁵ | 0.43 | −1.2 |

TABLE 24
Passaging parameters days 35-42

| | Day 35 | | | | | | Day 42 | | | | | |
|---|---|---|---|---|---|---|---|---|---|---|---|---|
| | | | Estimated | Harvesting | | | | | Estimated | Harvesting | | |
| | | | cell | density | | | | | cell | density | | |
| | Lactate | % | diameter | (live | | | Lactate | % | diameter | (live | | |
| Run | (mM) | viability | (μm) | cells/cm²) | Yield | PDL | (mM) | viability | (μm) | cells/cm²) | Yield | PDL |
| Ongoing | Aborted | | | | | | | | | | | |
| ICB | 2.36 | 99 | 13.3 | 2.11 × 10⁵ | 5.27 | 2.4 | 1.83 | 95 | 13.6 | 1.77 × 10⁵ | 4.43 | 2.1 |

Until day 14, all passaging parameters were in the range of previous successful runs (Report #OPC-REP-03). On the other hand, between days 14-21, massive clots formation was observed in the ongoing run, thus decreasing the number of aggregates and the lactate on day 21 to an average of 6.57 mM. As a result, the ongoing run was aborted, and cryopreserved cells were thawed and seeded as above. In the ICB thawed cells, clots formation was not observed and as a result, the lactate on day 21 was higher (average of 7.58 mM). Passaging parameters of days 28-42 were in the expected and acceptable range.

Markers expression—Markers expression of #20-EROPCRD-02 and #ER-ICBRD-02 are presented in Table 25.

TABLE 25
Markers expression on days 7-28

| | Day 7 (IPC 064) | | | | | | | | | | |
|---|---|---|---|---|---|---|---|---|---|---|---|
| | | | | | | | TRA- | Day 14 (IPC 066) | | | |
| Run | PDGFRα | NG2 | GD3 | A2B5 | Claudin 6 | EpCAM | 1-60 | PDGFRα | NG2 | GD3 | A2B5 |
| Ongoing | 2.01 | 0.33 | 99.60 | 41.80 | 59.10 | 95.10 | 7.04 | 60.60 | 28.30 | 99.60 | 98.90 |
| ICB | | | | | | | | | | | |

| | Day 14 (IPC 066) | | | | | | | | |
|---|---|---|---|---|---|---|---|---|---|
| | | | TRA- | Day 28 (IPC 068) | | | | | |
| Run | Claudin 6 | EpCAM | 1-60 | PDGFRα | NG2 | GD3 | A2B5 | Claudin 6 | EpCAM |
| Ongoing | 7.05 | 1.49 | 0.38 | Aborted | | | | | |
| ICB | | | | 93.30 | 81.20 | 97.10 | 51.40 | 0.39 | 0.49 |

TABLE 26
Markers expression on day 35

| | Day 35 (IPC 069) | | | | | | | | | | | |
|---|---|---|---|---|---|---|---|---|---|---|---|---|
| Run | PDGFRα | NG2 | PDGFRβ | GD3 | A2B5 | Claudin 6 | EpCAM | TRA-1-60 | CD49f | CK7 | β Tubulin III | GFAP |
| Ongoing | Aborted | | | | | | | | | | | |
| ICB | 97.90 | 84.00 | 95.90 | 95.50 | 33.80 | 0.08 | 0.56 | 0.09 | 1.13 | 0.11 | 0.42 | 0.04 |

As described in passaging parameters—markers expression until day 35 did not point to any major discrepancy from previous successful runs.

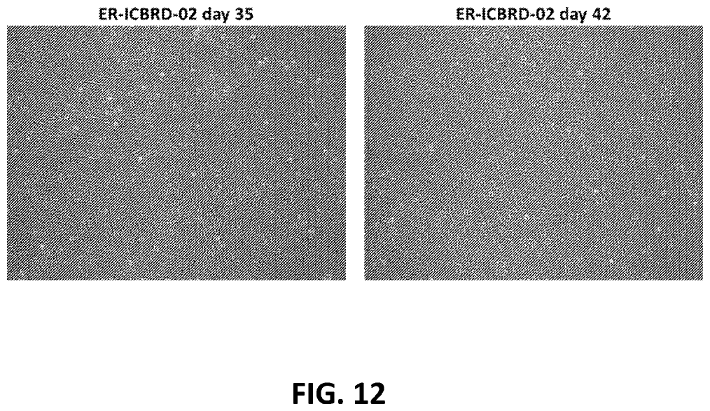

Morphology—Starting from day 35, the culture of successful and failed groups differs by different morphology. In FIG. 12, photos of #ER-ICBRD-02 are showing that the cells are organized as compact and dense cells and did not acquire the spindle-like ‘failed’ morphology. Batch Release—Summary of #ER-ICBRD-02 batch release testing according to Table 23 and presented in Table 27.

TABLE 27
Batch release tests for #ER-ICBRD-02

| Test | Method | Target tested | Proposed Criteria | | Result | |
|---|---|---|---|---|---|---|
| Viability | NC-200 | % of viable population | ≥70% | | %92 | |
| Strength | NC-200 | Live cells/ml | 0.75-1.25 × 10⁸ | | 0.98 × 10⁸ | |
| | | | Live cells/ml | | Live cells/ml | |
| | Biosafety | | | | | |
| Sterility | <USP71> | Turbidity | No growth | | No Growth | |
| Mycoplasma | <USP63> | Mycoplasma (CFU) | No growth | | Not tested | |
| Endotoxin | <USP85> | Endotoxin (EU/ml) | <2 | EU/ml | <1.98 | EU/ml |
| | Identity | | | | | |
| OPC | Flow | % GD3⁺ | ≥80% | | 93.4% | |
| | Cytometry | % NG2⁺ | ≥80% | | 92.3% | |
| | | % PDGFRa⁺ | ≥90% | | 98.2% | |
| | | % PDGFRβ⁺ | ≥90% | | 98.4% | |
| | Non-targeted cell populations | | | | | |
| Epithelial | Flow | % Keratin 7+ | ≤2% | | 0.43% | |
| | Cytometry | % of Claudin-6+ | ≤2% | | 0.05% | |
| | | % of EpCAM+ | ≤2% | | 1.62% | |
| | | % CD49f | ≤10% | | 0.39% | |
| hESCs Residual | | % TRA-1-60+ | ≤1% | | %0.00 | |
| | | % TRA-1-60+/Oct-4+ | FIO | | 0.00357% | |
| Mesoderm | | % GFAP | ≤2% | | 0.03% | |
| Astrocytes | | % CXCR4+/CD56+ | FIO | | Not tested | |
| Neuronal Phenotype | | % b-Tubulin III+ | FIO | | 0.19% | |
| Mesenchymal | qPCR | OLR1 | FIO | | Not tested | |
| Endoderm | | FOXA2, SOX17, AFP | FIO | | Not tested | |
| | Function/Potency | | | | | |
| Decorin Secretion | ELISA | Decorin (ng/ml) | ≥25 | ng/ml | 41 | ng/ml |

| | | | | |
|---|---|---|---|---|
| Cell Migration | NC-200 | % Migrated (PDGF-induced) | ≥30% | 58% |
| | | | (+PDGFββ) | |

Batch ER-ICBRD-02 cells met all the proposed acceptance criteria for batch release.

Discussion: Batch #20-EROPCRD-02 ongoing run and #ER-ICBRD-02 run thawed from ICB day 14 engineering runs, were produced in R&D labs as part of proof of the feasibility for full-scale LCTOPC1 batch production, and their product is designated for comparability and animal studies purposes.

In the #20-EROPCRD-02 ongoing run, processing multiple vessels simultaneously apparently caused the formation of an unusually higher number of clots in all PBS wheels and the termination of this run. Clots formation in the PBS wheel between days 14-21 harms the differentiation process due to aggregates loss and non-indicative lactate reading on day 21. In addition, cryopreservation and thawing of day 14 harvested cells (LCTOPC1 ICB), can decrease the number of clots formed on days 14-21 compared to the ongoing run of the same cells. Indeed, in the #ER-ICBRD-02 run, started from the ICB generated during #20-EROPCRD-02, clots were not observed in the PBS wheels. These differences between the ongoing and the ICB runs concluded that the cryopreservation and thawing of day 14 ICB are beneficial and necessary for the OPC1 process in the GMP facility.

Batch #ER-ICBRD-02 passed all proposed IPCs and batch release criteria in the tested assays, approving that manufacturing full-scale LCTOPC1 batches with cryopreservation of ICB on day 14 is feasible. This batch, #ER-ICBRD-02, is representative for the LCTOPC1 GMP process and released cells.

Conclusions: #ER-ICBRD-02 is a representative batch for the GMP process and released cells of LCTOPC1 and passed all proposed criteria for batch release. Production of full-scale OPC1 cells that meet the proposed criteria for batch release is feasible. Cryopreservation of ICB on day 14 and thawing before seeding for aggregates formation is beneficial for the cells and prevents clots formation.

### Example 6: Differentiation of Thawed Cells on Day 14 to Produce OPCs

The purpose of this example is to demonstrate establishment of differentiation conditions of thawed cells to OPCs.

Experimental Procedure: FIG. 13 shows the steps of the OPC differentiation protocol.

TABLE 28
Tested conditions:

| | | Cryopre- | | | | | |
|---|---|---|---|---|---|---|---|
| | | servation | | | | | |
| | | according | | Day 14 | | | |
| | | to | | Thawing | | Lactate | |
| | ICB | DEVOPC- | | recovery | Day 14 tested | day 21 | |
| Run # | origin | PRO-09 | Group | (%)* | characterizations | (mM) | Pass/Fail |
| 10.1 | RD#10 | Yes | A | 91 | Pre coating GPM | 23.28 | Pass |
| | grp.D | | | | over night | | |
| | | | B | 93 | Prewash with anti- | 22.70 | Fail |
| | | | | | adherence solution | | |
| ICB14#1 | RD#10 | Yes | B | 91 | Pre coating GPM | 21.47 | Pass |
| | grp.D | | | | over night | | |
| | | | C | 91 | Prewash with anti- | 21.22 | Pass |
| | | | | | adherence solution** | | |
| | RD#7 | No | D | 79 | Pre coating GPM | 15.41 | Pass |
| | grp.F3 | | | | over night | | |
| | RD#11 | No | E | 74 | NA | 20.09 | Pass |
| | grp.C | | | | | | |
| ICB14#2 | RD#12 | No | A | 72 | NA | 15.04 | Pass |
| | grp.A | | | | | | |

$* \% recovery of life cells = \frac{\# of live cells thawed}{\# of live cells cryopreserved} * 100 \%$ **Prewash with anti-adherence solution - anti adherent solution was thought to prevent from aggregates to adhere at the bottom of the wheel at days 14-16, later it turned out that the main reason for aggregates loss was their tendency to stick to each other which resulted in large clots creation.

HESCs culturing (stage 1)—H1 hESCs (PILOT-LCT-H1-002) were cultured for 3 passages on LN521 coated vessels. At the end of p30+6+4+2, cells were seeded for 4 days for OPC differentiation initiation.

OPC Differentiation—on day 4 of p30+6+4+3 of stage 1, OPC differentiation was initiated. Any changes tested are detailed in Table 28.

Days 0-3—cells were cultured in GPM medium supplemented with 2 μM Dorsomorphin, 10 μM PD0325901, and 1 μM RA, was changed daily.

Days 4-6—culturing in GPM medium supplemented with 150 μM AA, and 1 μM RA, was changed daily.

Day 7—cells were harvested with TS and seeded in GPM supplemented with 10 ng/ml EGF, 10 ng/ml hs-FGF, and 10 μM RI at 26,667 live cells/cm² on LN521 coated vessels.

Day 9-13—culturing in GPM medium supplemented with 10 ng/ml EGF, and 10 ng/ml hs-FGF was changed every 2 days.

Day 14—cells were harvested with TS. In ongoing culture, cells that were not seeded were cryopreserved in CS10.

ICB Day 14 thawing — cells were thawed, resuspended, counted and 128×10⁶ live cells were seeded in 70 ml GPM medium supplemented with 10 ng/ml EGF, 10 ng/ml hs-FGF, and 10 μM RI.

Day 16-20—culturing in 80% of GPM medium supplemented with 10 ng/ml EGF, and 10 ng/ml hs-FGF was changed every 2 days.

Day 21—aggregates were flattened according to lactate measurement (flattening on 75 cm² if lactate concentration was below 15.00 mM or 150 cm² if lactate concentration was equal or higher than 15.00 mM) on day 21 in GPM supplemented with 20 ng/ml EGF, and 10 ng/ml PDGF-AA on LN521 coated vessels.

Days 23-27—culturing in GPM medium supplemented with 20 ng/ml EGF, and 10 ng/ml PDGF-AA was changed every 2 days.

Day 28—cells were harvested with TS and seeded in GPM supplemented with 10 ng/ml EGF, and 10 ng/ml PDGF-AA at 40,000 live cells/cm² on LN521 coated vessels.

Days 29-34—culturing in GPM medium supplemented with 20 ng/ml EGF, and 10 ng/ml PDGF-AA was changed every 2 days.

Day 35—cells were harvested with TS and seeded in GPM supplemented with 10 ng/ml EGF, and 10 ng/ml PDGF-AA at 40,000 live cells/cm² on LN521 coated vessels.

Days 36-41—culturing as above. GPM medium supplemented with 20 ng/ml EGF, and 10 ng/ml PDGF-AA was changed every 2 days.

Day 42—Harvesting and cryopreservation of cells with TS.

QC tests—Flow cytometry—analysis of markers by FACS was performed on cryopreserved cells on days 7, 14, 28, 35, and 42 according to Table 29.

TABLE 29
FACS staining

| Category | Subcategory | Marker | Day 7 | Day 14 | Day 28 | Day 35 | Day 42 |
|---|---|---|---|---|---|---|---|
| Purity/ | OPC | PDGFRα | ✓ | ✓ | ✓ | ✓ | ✓ |
| Identity | | PDGFRβ | | | | ✓ | ✓ |
| | | NG2 | ✓ | ✓ | ✓ | ✓ | ✓ |
| | Pre-OPC | A2B5 | ✓ | ✓ | ✓ | ✓ | ✓ |
| | | GD3 | ✓ | ✓ | ✓ | ✓ | ✓ |

| Impurities/ | TRA-1-60 | ✓ | ✓ | | ✓ | ✓ |
|---|---|---|---|---|---|---|
| Non-targeted | Claudin 6 | ✓ | ✓ | ✓ | ✓ | ✓ |
| population | EpCAM | ✓ | ✓ | ✓ | ✓ | ✓ |
| | Keratin 7 | | | | ✓ | ✓ |
| | CD49f | | | | ✓ | ✓ |
| | Beta Tubulin III | | | | ✓ | ✓ |
| | GFAP | | | | | ✓ |

Decorin secretion—cryopreserved cells from day 35 and 42 were tested for Decorin secretion.

Acceptance criteria—as the assays for assessing OPC batches were developed alongside the process, successful batch was defined according to the following criteria: purity markers (PDGFRα)>95.00%, impurities markers (Claudin, EpCAM, CK7)<2.00%, decorin secretion (day 42 cells)>25.00 ng/ml, morphology—small and compact cells, absence of elongated spindle-like morphology.

Results: Passaging parameters—at each passage, yield and PDL values were calculated according to the following formulas:

$${Yield}_{days 35 , 42} = \frac{Harvesting density ( on day x ) [ live cells per {cm}^{2} ]}{Seeding density ( last passage ) [ live cells per {cm}^{2} ]} \\ {Yield}_{day 28} = \frac{Harvested cells ( on day 28 )}{Seeded cells ( on day 14 )} \\ PDL = {log}_{2} ( Yield )$$

TABLE 30
Passaging parameters days 21-35. All ICB 14 runs come below.

| | | | Day 28 | | | | | | Day 35 | | | | | |
|---|---|---|---|---|---|---|---|---|---|---|---|---|---|---|
| | | | | | Estimated | Harvesting | | | | | Estimated | Harvesting | | |
| | | Day 21 | | | cell | density | | | | | cell | density | | |
| | | Lactate | Lactate | % | diameter | (live | | | Lactate | % | diameter | (live | | |
| Run # | Group | (mM) | (mM) | Viability | (μm) | cells/cm2) | Yield | PDL | (mM) | Viability | (μm) | cells/cm2) | Yield | PDL |
| RD#10 | D | 23.01 | 18.51 | 71 | 11.75 | 792,000 | 0.93 | −0.1 | 2.38 | 97 | 14.1 | 198,200 | 4.96 | 2.3 |
| 10.1 | A | 23.28 | 19.27 | 66 | 11.45 | 686,667 | 0.80 | −0.3 | 2.54 | 97 | 14.45 | 142,667 | 3.57 | 1.8 |
| | B | 22.70 | 19.01 | 62 | 11.30 | 962,000 | 1.13 | 0.2 | 3.38 | 97 | 14.45 | 173,333 | 4.33 | 2.1 |
| ICB14#1 | B | 21.47 | 17.89 | 76 | 11.20 | 624,000 | 0.73 | −0.5 | 2.83 | 88 | 14.10 | 209,333 | 5.23 | 2.4 |
| | C | 21.22 | 17.86 | 82 | 11.30 | 507,000 | 0.59 | −0.8 | 2.68 | 91 | 14.20 | 186,667 | 4.67 | 2.2 |
| RD#7 | F3 | 14.69 | 11.31 | 92 | 12.70 | 878,000 | 1.03 | 0.0 | 3.84 | 97 | 14.30 | 261,333 | 6.53 | 2.7 |
| ICB14#1 | D | 15.41 | 13.48 | 90.4 | 12.55 | 963,000 | 1.13 | 0.2 | 2.44 | 98 | 14.00 | 185,333 | 4.63 | 2.2 |
| RD#11 | C | 21.99 | 18.66 | 88 | 12.40 | 734,000 | 0.86 | −0.2 | 2.16 | 98 | 14.00 | 184,000 | 4.60 | 2.2 |
| ICB14#1 | E | 20.09 | 18.06 | 81 | 12.45 | 575,000 | 0.71 | −0.5 | 1.87 | 96 | 13.90 | 223,333 | 5.58 | 2.5 |
| RD#12 | A | 11.59 | 14.11 | 84 | 12.90 | 1,218,000 | 0.71 | −0.5 | 1.53 | 97 | 14.20 | 126,667 | 3.17 | 1.7 |
| ICB14#2 | A | 15.04 | 13.42 | 87 | 13.40 | 812,000 | 0.95 | −0.1 | 1.54 | 97 | 14.10 | 182,667 | 4.57 | 2.2 |

TABLE 31
Passaging parameters day 42. All ICB 14 runs come below.

| | | Day 42 | | | | | |
|---|---|---|---|---|---|---|---|
| | | | | Estimated | Harvesting | | |
| | | Lactate | % | cell diameter | density (live | | |
| Run # | Group | (mM) | Viability | (μm) | cells/cm²) | Yield | PDL |
| RD#10 | D | 1.11 | 97 | 14.25 | 132,000 | 3.30 | 1.7 |
| 10.1 | A | 1.31 | 99 | 14.1 | 130,889 | 3.27 | 1.7 |
| | B | 1.72 | 98 | 14.15 | 125,000 | 3.13 | 1.6 |
| ICB14#1 | B | 1.29 | 97 | 14.8 | 115,111 | 2.88 | 1.5 |
| | C | 1.25 | 99 | 14.3 | 114,000 | 2.85 | 1.5 |
| RD#7 | F3 | 1.75 | 99 | 15 | 139,556 | 3.49 | 1.8 |
| ICB14#1 | D | 1.71 | 99 | 14.4 | 143,111 | 3.58 | 1.8 |
| RD#11 | C | 0.91 | 97 | 13.9 | 148,741 | 3.72 | 1.9 |
| ICB14#1 | E | 1.19 | 98 | 13.9 | 153,200 | 3.83 | 1.9 |
| RD#12 | A | 0.98 | 99 | 14.0 | 174,000 | 4.35 | 2.1 |
| ICB14#2 | A | 1.30 | 98 | 13.9 | 135,600 | 3.39 | 1.8 |

Some similarities can be found between RD runs passaging parameters and their derivatives, ICB runs, mainly at day 21 and 28—in lactate concentration; all other parameters showed acceptable values. The lower viability in Run #10.1 groups on day 28 is related to manual handling manipulation.

Markers expression—at each passage, yield and PDL values were calculated according to the following formulas:

TABLE 32
Markers expression on days 28. All ICB 14 runs come below.

| | | Day 28 | | | | | | | |
|---|---|---|---|---|---|---|---|---|---|
| | | | | PDGFRα/ | | | | | TRA- |
| Run # | Group | PDGFRα | NG2 | NG2 | GD3 | A2B5 | Claudin 6 | EpCAM | 1-60 |
| RD#10 | D | 84.20 | 70.30 | 64.50 | 98.40 | 98.40 | 2.09 | 0.22 | NA |
| 10.1 | A | 57.80 | 72.00 | 52.70 | 90.00 | 90.10 | 1.10 | 0.61 | 0.49 |
| | B | 49.50 | 58.50 | 43.70 | 95.10 | 95.10 | 0.85 | 0.61 | 0.21 |
| ICB14#1 | B | 73.70 | 89.80 | 70.30 | 95.30 | 95.30 | 1.28 | 1.03 | NA |
| | C | 63.50 | 80.00 | 60.20 | 97.20 | 95.70 | 1.29 | 1.16 | NA |
| RD#7 | F3 | 93.11 | 93.11 | NA | NA | NA | 1.18 | 1.04 | NA |
| ICB14#1 | D | 89.30 | 93.20 | 85.60 | 95.30 | 65.20 | 1.77 | 1.23 | NA |
| RD#11 | C | 81.30 | 87.50 | 76.40 | 95.60 | 90.20 | 7.83 | 0.47 | 0.44 |
| ICB14#1 | E | 75.00 | 84.40 | 70.80 | 95.70 | 92.20 | 5.32 | 1.58 | NA |
| RD#12 | A | 88.30 | 88.00 | 81.70 | 93.60 | 65.80 | 2.54 | 0.4 | NA |
| ICB14#2 | A | 96.80 | 87.90 | 86.80 | 98.30 | 63.10 | 2.09 | 0.28 | NA |

TABLE 33
Markers expression on days 35. All ICB 14 runs come below.
Day 35

| | | | | PDGFRα/ | | | | | | TRA- | | | Beta | |
|---|---|---|---|---|---|---|---|---|---|---|---|---|---|---|
| Run # | Group | PDGFRα | NG2 | NG2 | PDGFRβ | GD3 | A2B5 | Claudin 6 | EpCAM | 1-60 | CD49f | CK7 | tub3 | GFAP |
| RD#10 | D | 93.00 | 92.40 | 88.90 | 95.30 | 99.40 | 65.10 | 0.38 | 0.88 | 0.29 | 4.89 | 0.04 | 0.67 | 0.17 |
| 10.1 | A | 92.50 | 82.60 | 75.37 | 96.60 | 98.80 | 65.20 | 0.46 | 0.57 | 0.27 | 4.97 | 0.10 | 2.66 | 0.10 |
| | B | 86.00 | 71.70 | 60.40 | 95.30 | 98.20 | 64.40 | 0.90 | 0.84 | 0.38 | 5.71 | 0.09 | 2.38 | 0.23 |
| ICB14#1 | B | 87.20 | 87.20 | 83.00 | 93.30 | 98.10 | 92.90 | 0.24 | 0.36 | 0.14 | 4.62 | 0.07 | 0.70 | 0.31 |
| | C | 90.20 | 90.20 | 87.60 | 95.00 | 98.00 | 92.90 | 0.25 | 0.41 | 0.18 | 3.93 | 0.12 | 1.22 | 0.47 |
| RD#7 | F3 | 98.10 | 95.60 | 94.00 | NA | NA | 94.5 | NA | 0.24 | 0.41 | NA | NA | NA | NA |
| ICB14#1 | D | 89.00 | 89.00 | 87.60 | 91.40 | 95.80 | 38.90 | 0.17 | 0.56 | 0.16 | 1.84 | 0.44 | 3.15 | 0.46 |
| RD#11 | C | 96.30 | 97.90 | 94.90 | 98.00 | 98.50 | 80.00 | 0.35 | 0.54 | 0.26 | 4.93 | 0.16 | 0.88 | 0.09 |
| ICB14#1 | E | 91.70 | 91.70 | 90.80 | 97.70 | 97.50 | 70.80 | 0.18 | 0.33 | 0.15 | 2.91 | 0.22 | 1.71 | 0.59 |
| RD#12 | A | 93.80 | 91.40 | 87.50 | 98.40 | 93.40 | 31.10 | 0.18 | 0.54 | 0.17 | 2.40 | 0.07 | 2.30 | 0.06 |
| ICB14#2 | A | 97.60 | 94.20 | 93.10 | 97.60 | 95.80 | 34.10 | 0.07 | 0.48 | 28.6 | 1.47 | 0.02 | 2.81 | 0.03 |

TABLE 34
Markers expression on day 42. All ICB 14 runs come below.
Day 42

| | | | | NG2/ | | | | | | TRA- | | | beta | |
|---|---|---|---|---|---|---|---|---|---|---|---|---|---|---|
| Run # | Group | PDGFRa | NG2 | PDGFRa | PDGFRb | GD3 | A2B5 | Claudin 6 | EpCAM | 1-60 | CD49f | CK7 | tub3 | GFAP |
| RD#10 | D | 97.20 | 78.00 | 76.80 | 98.70 | 93.80 | 31.90 | 0.26 | 0.46 | 0.26 | 3.31 | 1.00 | 1.84 | 0.08 |
| 10.1 | A | 96.80 | 91.10 | 88.50 | 98.10 | 95.70 | 22.70 | 0.41 | 0.83 | 0.24 | 3.04 | 0.32 | 1.38 | 0.10 |
| | B | 94.90* | 89.50 | 85.70 | 96.30 | 93.30 | 32.80 | 0.40 | 0.69 | 0.36 | 5.78 | 0.40 | 2.51* | 0.03 |
| ICB14#1 | B | 97.20 | 91.60 | 89.50 | 98.60 | 88.90 | 49.60 | 0.18 | 0.45 | 0.10 | 1.74 | 0.27 | 1.66 | 0.14 |
| | C | 97.80 | 92.60 | 90.70 | 99.10 | 91.00 | 65.00 | 0.17 | 0.56 | 0.24 | 1.44 | 0.27 | 1.97 | 0.04 |
| RD#7 | F3 | 98.60 | 95.90 | 97.10 | 97.40 | 90.90 | 7.79 | 0.15 | 1.39 | NA | 2.27 | 1.16 | 1.69 | 0.11 |
| ICB14#1 | D | 97.30 | 92.20 | 89.80 | 97.70 | 89.20 | 26.90 | 0.17 | 0.67 | 0.14 | 0.9 | 0.87 | 3.06 | 0.07 |
| RD#11 | C | 98.40 | 90.50 | 89.10 | 98.70 | 92.60 | 57.80 | 0.42 | 0.72 | 0.56 | 1.26 | 0.38 | 1.02 | 0.09 |
| ICB14#1 | E | 98.70 | 92.30 | 91.10 | 99.40 | 85.20 | 27.30 | 0.18 | 0.49 | 0.18 | 1.12 | 0.26 | 3.76 | 0.16 |
| RD#12 | A | 99.00 | 90.70 | 90.40 | 99.10 | 86.80 | 16.50 | 0.15 | 0.99 | 0.1 | 2.47 | 0.57 | 1.79 | 0.56 |
| ICB14#2 | A | 98.60 | 88.00 | 88.00 | 99.60 | 86.80 | 8.06 | 0.07 | 0.45 | 0.29 | NA | 0.15 | 0.20 | 0.03 |

*Failed run markers expression is out of acceptance criteria range.

All ICB groups beside run 10.1 group B met the proposed acceptance criteria for markers expression on day 42, indicating that cryopreservation and thawing procedures on day 14, do not harm the cells or the OPC1 differentiation process. As for run 10.1 group B, the PBS wheel treatment with anti-adherence solution did not improve the process and will not be qualified for the final OPC1 process in the GMP facility.

Potency assay — Decorin secretion of on days 35 and 42 is presented in Table 35.

TABLE 35
Decorin secretion. All ICB 14 runs come below.

| Run # | Group | Day 42 - Decorin secretion (ng/ml) |
|---|---|---|
| RD#10 | D | 39.40 |
| 10.1 | A | 27.45 |
| | B | 32.44 |
| ICB14#1 | B | 48.35 |
| RD#7 | F3 | 26.05 |
| ICB14#1 | D | 30.41 |
| RD#11 | C | 29.12 |
| ICB14#1 | E | 40.35 |
| RD#12 | A | 28.54 |
| ICB14#2 | A | 31.48 |

Decorin secretion in all groups was >25 ng/ml on day 42 and met the expected criteria.

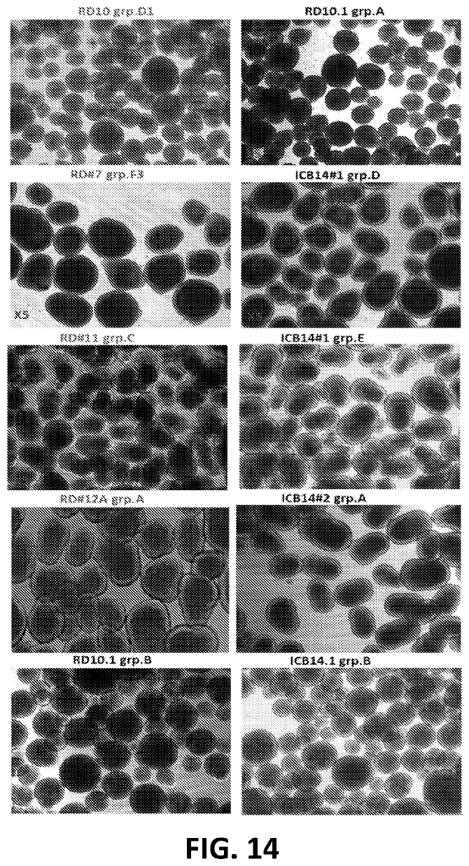

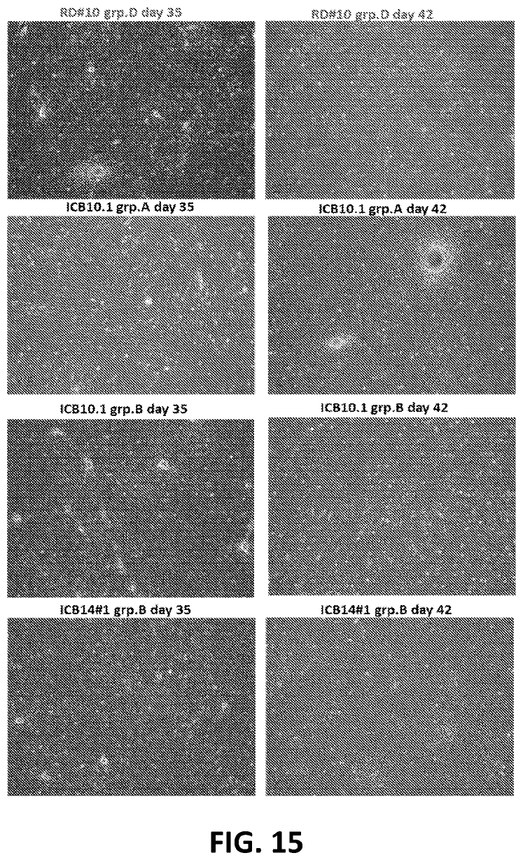

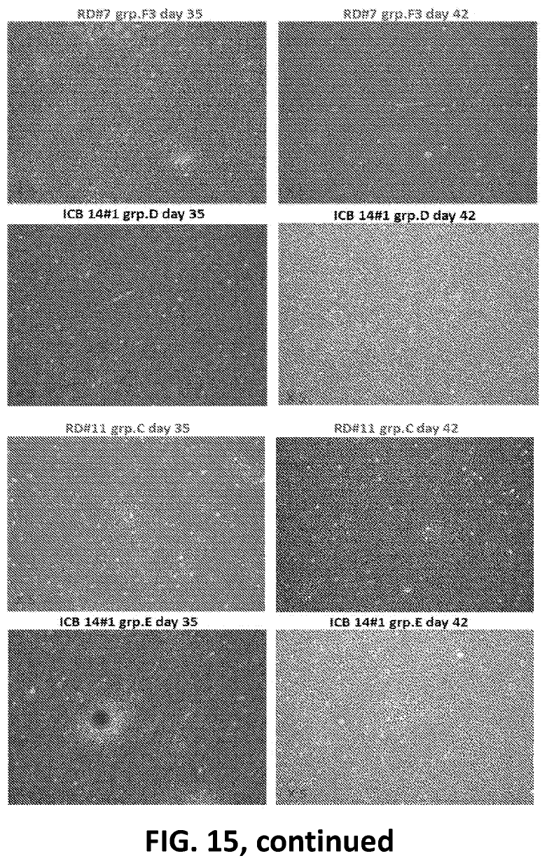

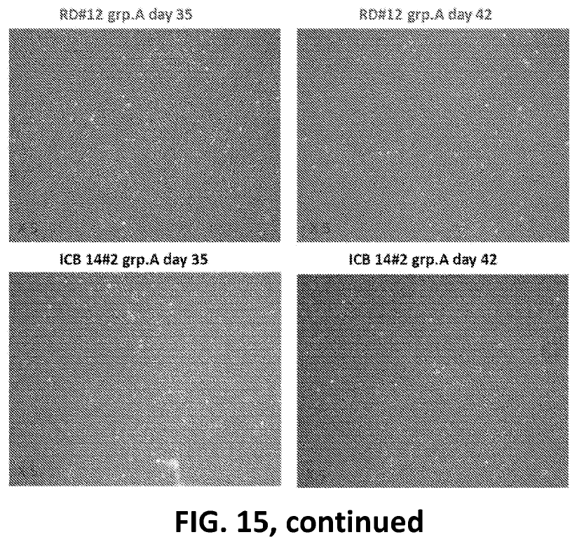

Morphology—starting from day 35, the culture of successful and failed groups differs by different morphology. In FIG. 14 and FIG. 15, photos of morphology of ongoing runs are presented compared to the ICB thawed cells. On day 35 and 42 the cells are organized as compact and dense cells and did not acquire the spindle-like ‘failed’ morphology.

Clear day 21 aggregates morphology resemblance between origin runs and their derived ICB's is presented in FIG. 14.

Discussion: Cryopreservation of ICB on day 14 during OPC1 differentiation process holds a great potential advantages such as reduction of clots formation in the PBS wheel that resulted in runs failure, production of multiple batches from the same ICB and allows flexibility of the 9 weeks long process. Results showed that OPC1 cells produced from both ongoing full runs and their day 14 ICB derived runs acquired the expected criteria for OPC population.

In this report, the results of differentiation of thawed cells on day 14 are summarized relative to the origin ongoing group. Passaging parameters, markers expression and potency assay (Decorin secretion) showed that beside run 10.1 group B, all cells thawed on day 14 met the proposed acceptance criteria for OPC1 cells. As for run 10.1 group B, the PBS wheel was pretreated with anti-adherence solution before seeding cells on day 14. As this treatment did not show a clear benefit for the process compared to pre-loading with GPM, it was decided not to use this material in the process. In addition, it was found that when the ICB cryopreservation procedure described herein is implemented, % recovery was improved from 75% to 92% (see Table 28). The procedure will be performed in CCN OPC1 process.

Conclusions: Day 14 can be a breakpoint for the OPC1 differentiation protocol. Day 14 ICB cells can be thawed directly into PBS wheels and continue with the established process of the ongoing protocol. Day 14 ICB shows clear advantage in reducing sediments in the Days 14-21 aggregates step. Better post thawing recovery at day 14 thawing was obtained when cryopreservation was done according to methods described herein.

### REFERENCES

Duester, G. (2008). “Retinoic acid synthesis and signaling during early organogenesis.” Cell 134(6): 921-931.
Hu, B. Y., Z. W. Du, X. J. Li, M. Ayala and S. C. Zhang (2009). “Human oligodendrocytes from embryonic stem cells: conserved SHH signaling networks and divergent FGF effects.” Development 136(9): 1443-1452.
Janesick, A., S. C. Wu and B. Blumberg (2015). “Retinoic acid signaling and neuronal differentiation.” Cell Mol Life Sci 72(8): 1559-1576.
Koch, U., R. Lehal and F. Radtke (2013). “Stem cells living with a Notch.” Development 140(4): 689-704.
Kudoh, T., S. W. Wilson and I. B. Dawid (2002). “Distinct roles for Fgf, Wnt and retinoic acid in posteriorizing the neural ectoderm.” Development 129(18): 4335-4346.
Li, Y. and M. M. Parast (2014). “BMP4 regulation of human trophoblast development.” Int J Dev Biol 58(2-4): 239-246.
Ota, M. and K. Ito (2006). “BMP and FGF-2 regulate neurogenin-2 expression and the differentiation of sensory neurons and glia.” Dev Dyn 235(3): 646-655.
Patthey, C. and L. Gunhaga (2014). “Signaling pathways regulating ectodermal cell fate choices.” Exp Cell Res 321(1): 11-16.
Sui, L., L. Bouwens and J. K. Mfopou (2013). “Signaling pathways during maintenance and definitive endoderm differentiation of embryonic stem cells.” Int J Dev Biol 57(1): 1-12.
Watabe, T. and K. Miyazono (2009). “Roles of TGF-beta family signaling in stem cell renewal and differentiation.” Cell Res 19(1): 103-115.
Zheng, W., Q. Li, C. Zhao, Y. Da, H. L. Zhang and Z. Chen (2018). “Differentiation of Glial Cells From hiPSCs: Potential Applications in Neurological Diseases and Cell Replacement Therapy.” Front Cell Neurosci 12: 239.

All references (including all non-patent literature, patents, and patent publications) provided herein are incorporated herein by reference in their entireties.

What is claimed is:

1. A method for obtaining a population of oligodendrocyte progenitor cells (OPCs) from undifferentiated pluripotent stem cells, the method comprising:
a) obtaining a culture of undifferentiated pluripotent human embryonic stem cells (hESCs);
b) culturing the undifferentiated pluripotent hESCs for a first time period under culture conditions sufficient to induce differentiation of the hESCs to neuroectoderm cells and to neural progenitor cells; and
c) culturing the neural progenitor cells from step b) for a second time period under culture conditions sufficient to differentiate the neural progenitor cells to OPCs.

2. The method of claim 1, wherein the pluripotent cells in step b) are cultured on laminin in an adherent tissue culture vessel, or in a suspended complex, or both.

3. The method of claim 1, wherein the pluripotent stem cells are human embryonic stem cells (hESCs).

4. The method of claim 1, wherein the pluripotent stem cells are human induced pluripotent stem cells (hiPSCs).

5. The method of claim 1, wherein the undifferentiated hESCs of step b) are cultured in the presence of Dorsomorphin, PD0325901, and RA.

6. The method of claim 5, further comprising the step of culturing in the presence of AA and RA.

7. The method of claim 1, wherein the neuroectoderm cells from step b) are cultured in the presence of EGF and hsbFGF.

8. The method of claim 7, further comprising the step of culturing in the presence of EGF and PDGF-AA.

9. The method of claim 1, wherein the first time period is from about 3 days to about 60 days.

10. The method of claim 9, wherein the first time period is from about 10 days to about 15 days.

11. The method of claim 9 wherein the first time period is about 14 days.

12. The method of claim 1, wherein the second time period is from about 10 days to about 60 days.

13. The method of claim 12, wherein the second time period is from about 20 days to about 40 days.

14. The method of claim 12, wherein the second time period is about 28 days.

15. The method of claim 1, wherein the differentiated hESCs in step b) are cryopreserved at about day 14.

16. The method of claim 15 wherein the cryopreserved cells are thawed, and wherein the subsequently thawedcells are cultured in any remaining steps of the method.

17. The method of claim 15, comprising the step of cryopreserving the neural progenitor cells from step b) at or about the completion of the first time period.

18. The method of claim 16 wherein the cryopreserved neuroectoderm cells are thawed and cultured in accordance with step c).

19. The method of claim 14, wherein the OPCs of step c) are cryopreserved.

20. The method of claim 19 wherein the cryopreserved OPCs are thawed.

21. The method of claim 1, wherein the OPCs express one or more markers selected from neural/glial antigen 2 (NG2), platelet-derived growth factor receptor A (PDGFRα), and platelet-derived growth factor receptor B (PDGFRβ).

22. A method of formulating an oligodendrocyte progenitor cells (OPCs) composition for administration to a subject directly after thawing, said method comprising: (a) suspending the OPCs according to claim 1 in a cryopreservation media to form a cell suspension, (b) storing the cell suspension at a cryopreservation temperature, and (c) thawing the cryopreserved suspension.

23. The method of claim 22, wherein the cryopreservation media comprises one or more of adenosine, dextran-40, lactobionic acid, HEPES (N-(2-Hydroxyethyl) piperazine-N′-(2-ethanesulfonic acid)), sodium hydroxide, L-glutathione, potassium chloride, potassium bicarbonate, potassium phosphate, dextrose, sucrose, mannitol, calcium chloride, magnesium chloride, potassium hydroxide, sodium hydroxide, dimethyl sulfoxide (DMSO), and water.

24. A pharmaceutical composition for administration to a subject, said composition comprising the OPCs according to claim 1 and a cryopreservation media.

25. The pharmaceutical composition of claim 24, wherein the cryopreservation media comprises one or more of adenosine, dextran-40, lactobionic acid, HEPES (N-(2-Hydroxyethyl) piperazine-N′-(2-ethanesulfonic acid)), sodium hydroxide, L-glutathione, potassium chloride, potassium bicarbonate, potassium phosphate, dextrose, sucrose, mannitol, calcium chloride, magnesium chloride, potassium hydroxide, sodium hydroxide, dimethyl sulfoxide (DMSO), and water.

26. A method for treating a spinal injury in a subject, the method comprising administering to said subject a therapeutically effective amount of the composition according to claim 22.

27. he method of claim 26, wherein the administering comprises administering the composition into or adjacent to a spinal cord injury site.

28. The method of claim 26 wherein administering is by injection.

29. The method of claim 26 wherein the administering is by implantation.

30. The method of claim 26 wherein the administering is by transplantation.

31. The pharmaceutical composition of claim 26, wherein the concentration of cells is about 1×10⁶ cells per mL to about 100×10⁶ cells per mL.

32. The pharmaceutical composition of claim 26, wherein the pharmaceutical composition is stored at a volume of about 100 microliters to about 1 milliliter.

33. The pharmaceutical composition of claim 26, wherein the concentration of cells is 100×10⁶ cells per mL.

34. The pharmaceutical composition of claim 26, wherein the pharmaceutical composition is stored at a volume of 250 microliters.

35. The pharmaceutical composition of claim 26, wherein the pharmaceutical composition is stored at a volume of 300 microliters.

36. The pharmaceutical composition of claim 26, wherein the cryopreservation media is a cryosolution.

37. The pharmaceutical composition of claim 26, wherein the cryosolution is CryoStor10 (CS10).

38. The pharmaceutical composition of claim 26, wherein the OPCs express one or more markers selected from neural/glial antigen 2 (NG2), platelet-derived growth factor receptor A (PDGFRα), and platelet-derived growth factor receptor B (PDGFRβ).
